SEQUENCE LISTING (110 sequences)

SEQ ID NO: 1 - protein, 118 aa, synthetic construct
  source 1..118: note = 74804_01E23A VH
QVQLVQSGAEVKKPGASVKVSCKASGYTFTGYYMHWVRQAPGQGLEWMGWLNPNSGGTDYVQKFQGRVTMTRDTSISTAYMELSRLRSDDTAVYYCARTLYYYGMDVWGQGTTVTVSS

SEQ ID NO: 2 - protein, 118 aa, synthetic construct
  source 1..118: note = 74804_30O16A VH
QVQLVQSGAEVKRPGASVKVSCKASGYTFTGYYMHWVRQAPGQGLEWMGWINPYSGGTDYAQKFQDRVTMTRDTSISTAYMELSRLRSDDTAVYYCARSYYYYGMDVWGQGITVTVSS

SEQ ID NO: 3 - protein, 118 aa, synthetic construct
  source 1..118: note = 74804_09H05A VH
QVQLFQSGAEVKKPGASVKVSCKASGYTFTGYYVHWVRQAPGQGLEWMGWINPNSGGTDYAQKFQGRVTMTWDTSISTAYMELSRLRSDDTAVYYCARSYYYYGMDVWGQGTTVTVSS

SEQ ID NO: 4 - protein, 122 aa, synthetic construct
  source 1..122: note = 74804_18L05A VH
QVQLVQSGAEVKKPGASVKVSCKTSGYTFTSCCMHWVRQAPGQGLEWMGIINPSVGSTSYAQKFQGIVTMTRDTSTSTVYMELSSLRSEDTAVYYCAVDSSGSRYYYGMDVWGQGTTVTVSS

SEQ ID NO: 5 - protein, 119 aa, synthetic construct
  source 1..119: note = 74804_07K10A VH
QVQLLQSGAEVKKPGSSVKVSCRASGGTFSIYAIDWVRQAPGQGLEWMGGIIPISGTENSAQNFQDRITITADKSTNTAYMELSSLRSEDTAVYYCARNGATSDAFDIWGQGTMVTVSS

SEQ ID NO: 6 - protein, 119 aa, synthetic construct
  source 1..119: note = 74804_12A21B VH and 74804_29D13A VH
QVQLLQSGAEVKKPGSSVKVSCKASGGTFSIYAIDWVRQAPGQGLEWMGGIIPIFGTANSAQKFQGRVTITADKSTSTAYMELSSLRSEDTAVYYCARNGATSDAFDMWGQGTMVTVSS

SEQ ID NO: 7 - protein, 119 aa, synthetic construct
  source 1..119: note = 74804_43F04A VH
QVQLLQSGAEVKKPGSSVKVSCKASGGTFSIYAIDWVRQAPGQGLEWMGGIIPIFGTENSAQNFQGRITITADKSTNTAYMELSSLRSEDTAVYYCARNGATSDAFDIWGQGTMVTVFS

SEQ ID NO: 8 - protein, 119 aa, synthetic construct
  source 1..119: note = 74804_51C10A VH
QVQLLQSGAEVKKPGSSVKVSCKASGGTFNIYAIDWVRQAPGQGLEWMGGIIPIFGTANSAQKFQGRVTITADKSTNTAYMELSSLRSEDTAVYYCARNGATSDAFDIWGQGTMVTVSS

SEQ ID NO: 9 - protein, 119 aa, synthetic construct
  source 1..119: note = 74804_25H09A VH
QVQLVQSGAEVKKPGSSVKVSCKASGDTFSNYAITWVRQAPGQGLEWIVGFSPIFGTANYAQKFQGRVTITADKSTSTAYMELSSLISEDTAVYYCAWGSGNFNWFDPWGQGTLVTVSS

SEQ ID NO: 10 - protein, 119 aa, synthetic construct
  source 1..119: note = 74804_05B24A VH
EVQLVDSGGGLVKPGGSLRLSCAATGFTFSNVWMSWVRQGSGKGLEWVGRIKSKTDGGTIDYAAPVKGRFTISRDDPKNTLYLQMNSLKTEDTAVYYCTTGELLGFDYWGQGTLVTVSS

SEQ ID NO: 11 - protein, 119 aa, synthetic construct
  source 1..119: note = 74804_45C18A VH
EVQLVESGGGLVKPGGSLRLACAATGFTFSNVWMSWVRQGSGKGLEWVGRIKSKADGGTIDYAAPVKGRFTISRDDSKNTLYLQMNSLKTEDTAMYYCTTGELLGFDYWGQGTLVTVSS

SEQ ID NO: 12 - protein, 118 aa, synthetic construct
  source 1..118: note = 74804_21D11A VH
EVQVVESGGGLVKPGGSLRLSCAASGFIFSKAWMSWVRQAPGKGLEWVGRIKSKTDGGTTDYAAPVKGRFTISRDDSKNTLYLQMNSLKTEDTAVYYCTTGGYVFDYWGQGALVTVSS

SEQ ID NO: 13 - protein, 121 aa, synthetic construct
  source 1..121: note = 74804_27I24A VH
EVQLVESGGGLVKPGGSLRLSCAASGFTFITYSMNWVRQAPGKGLEWVSSISSISSYIYYADSVKGRFTISRDNAKNSLYLQMDSLRAEDSAVYYCAREGIVGPTGYFDSWGQGTLVTVSS

SEQ ID NO: 14 - protein, 117 aa, synthetic construct
  source 1..117: note = 74804_16M09A VH
QVQLQESGPGLVKPSETLSLTCTVSGGSISSYFWNWIRQPPGKGLEWIGYGHYSGNSNHNPSLKSRVTISVDMSKNQFSLKMSSVTAADTAMYYCAREGLHDAFDIWGQGTMVTVSS

SEQ ID NO: 15 - protein, 117 aa, synthetic construct
  source 1..117: note = 74804_44J14A VH
QVQLQESGPGLVKPSETLSLTCTVSGGSVFSYFWNWIRQPPVKGLEWIGYIDHSGGTNYNPSLKSRVTMSVDTSKNQFSLKLSSVTAADTAVYYCAREGLHDAFDIWGQGTLVTVSS

SEQ ID NO: 16 - protein, 117 aa, synthetic construct
  source 1..117: note = 74804_01N02A VH
EVQLVQSGAEVKKPGESLKISCKGSGYSFTSYWIVWVRQMPGKGLEWMGFIYPGNSDTRYSPSFQGQVTISADQSISTAYLQWSSLKASDTATYYCARGGSYYLDYWGQGTLVTVSS

SEQ ID NO: 17 - protein, 118 aa, synthetic construct
  source 1..118: note = 74804_17E20A VH
EVQLVQSGAEVKKPGESLKISCKGSGYSFTSYWIGWVRQMPGKGLEWMGIIWPGDSDTRYSPSFQGQVTISADKSVNTAYLQWISLKASDTAIYYCARSSGGTAMDVWGQGTTVTVSS

SEQ ID NO: 18 - protein, 113 aa, synthetic construct
  source 1..113: note = 74804_01E23A VL and 74804_01N02A VL
DIVMTQSPDSLAVSLGERATIYCKSSQSVLINSNNQNYLAWYQQKPGQPPKLLIQWASTRESGVPDRFSGSGSGTDFTLTISSLQAEDVAVYYCQQYYNSPFTFGGGTKVEIK

SEQ ID NO: 19 - protein, 113 aa, synthetic construct
  source 1..113: note = 74804_30O16A VL
DIVMTQSPDSLAVSLGERATINCKSSQNIINSSTNKNYLAWYKQKPGQPPKLLIYWASTRESGVPDRFSGSGSGTDFTLTISSLQAEDVAVYYCQQYYSTPFTFGGGTKVEIK

SEQ ID NO: 20 - protein, 113 aa, synthetic construct
  source 1..113: note = 74804_09H05A VL
DIVMTQSPDSLAVSLGERATINCKSSQSVLYSSNNTSYLAWYQQKPGQPPKFLIYWTSTRESGVPDRFSGSGSGTDFTLTISSLQAEDVAVYYCQQYYITPFTFGPGTKVDIK

SEQ ID NO: 21 - protein, 107 aa, synthetic construct
  source 1..107: note = 74804_18L05A VL
EIVMTQSPATLSVSPGERATLSCRASQSVRSNLAWYQQRPGQAPRLLIYGTSTRATGIPARFSGSGSGTEFTLTISSLQSEDFAVYYCQQYNYWPYTFGQGTKLEIK

SEQ ID NO: 22 - protein, 108 aa, synthetic construct
  source 1..108: note = 74804_07K10A VL, 74804_29D13A VL and 74804_43F04A VL
EIVLTQSPGTLSLSPGERATLSCRASLSVSSNYLAWFQQRPGQAPRLLIYGVSRRATGIPDRFSGSGSGTDFTLTISRLEPEDFAVYYCQQYGSSPLTFGGGTKVEIK

SEQ ID NO: 23 - protein, 108 aa, synthetic construct
  source 1..108: note = 74804_12A21B VL
EIVLTQSPGTLSLSPGERATLSCRASLSVSSNYLAWFQQRPGQAPRLLIHGVSRRATGIPDRFSGSGSGTDFTLTISRLEPEDFAVYYCQQYGSSPLTFGGGTKVEIK

SEQ ID NO: 24 - protein, 108 aa, synthetic construct
  source 1..108: note = 74804_51C10A VL
EIVLTQSPGTLSLSPGERATLSCRASQSVSSNYLAWFQQRPGQAPRLLIYGVSRRATGIPDRFSGSGSGTDFTLTISRLEPEDFAVYYCQQYGSSPLTFGGGTKVEIK

SEQ ID NO: 25 - protein, 107 aa, synthetic construct
  source 1..107: note = 74804_25H09A VL
AIRMTQSPSSFSASTGDRVTITCRASQGISSYLAWYQQKPGKAPKLLIYAASTLQSGVPSRFSGSGSGTDFTLTISSLQSEDFATYYCQQYYSYPLTFGGGTKVEIK

SEQ ID NO: 26 - protein, 112 aa, synthetic construct
  source 1..112: note = 74804_05B24A VL
DIVMTQSPLSLPVTPGEPASISCRSGQSLLHNNGFNCLAWYLQKPGQSPQLLIYLGSNRASGVPDRFSGSGSGTDFTLKISRVEAEDVGVFYCMQTLQTPITFGQGTRLEIK

SEQ ID NO: 27 - protein, 112 aa, synthetic construct
  source 1..112: note = 74804_45C18A VL
DIVMTQSPLSLPVTPGEPASISCRSGQSLLHSNGFNCLAWYLQKPGQSPQLLIYLGSNRASGVPDRFSGSGSGTDFTLKISRVEAEDVGVYYCMQTLQTPITFGQGTRLEIK

SEQ ID NO: 28 - protein, 110 aa, synthetic construct
  source 1..110: note = 74804_21D11A VL
QSVLTQPPSASGTPGQRVTISCSGNNSNIGSYIVNWFQQLPGTAPKLLIYSKNQRPSGVPDRFSGSKSGTSASLAISGLQSEDEADYYCAAWDDSLNGVVFGGGTKLTVL

SEQ ID NO: 29 - protein, 111 aa, synthetic construct
  source 1..111: note = 74804_27I24A VL
QSVLTQPPSVSAAPGQKVTISCSGSSSNIGNHYVSWYQQLPGTAPKLLIYDNNKRPSGIPDRFSGSKSGTSATLGITGLQTGDEADYYCGTWDSRLSAYWVFGGGTKLTVL

SEQ ID NO: 30 - protein, 110 aa, synthetic construct
  source 1..110: note = 74804_16M09A VL
QSVLTQPPSASGTPGQRVTISCSGSISNIGSNTVNWYQQLPGTAPKLLIYSNNQRPSGVPDRFSGSKSGTSASLAISGLQSEDEADYYCAAWDDSLNGRVFGGGTKLTVL

SEQ ID NO: 31 - protein, 110 aa, synthetic construct
  source 1..110: note = 74804_44J14A VL
QSVLTQPPSASGTPGQRVTISCSGSSSNIGSNTVNWYQLLPGTAPKLLIYSNYQRPSGVPDRFSGSKSGTSASLAISGLQSEDEADYYCAAWDDSLNGRVFGGGTKLTVL

SEQ ID NO: 32 - protein, 112 aa, synthetic construct
  source 1..112: note = 74804_17E20A VL
DIVMTQSPLSLPVTPGEPASISCRSSQSLLHSNGYNTLDWYLQKPGQSPQLLIYLGSNRASGVPDRFSGSGSGTDFTLKISRVEAEDVGLYYCMQGLQTQWTFGQGTRVEIK

SEQ ID NO: 33 - protein, 8 aa, synthetic construct
  source 1..8: note = HCDR1
GYTFTGYY

SEQ ID NO: 34 - protein, 8 aa, synthetic construct
  source 1..8: note = HCDR1
GYTFTSCC

SEQ ID NO: 35 - protein, 8 aa, synthetic construct
  source 1..8: note = HCDR1
GGTFSIYA

SEQ ID NO: 36 - protein, 8 aa, synthetic construct
  source 1..8: note = HCDR1
GGTFNIYA

SEQ ID NO: 37 - protein, 8 aa, synthetic construct
  source 1..8: note = HCDR1
GDTFSNYA

SEQ ID NO: 38 - protein, 8 aa, synthetic construct
  source 1..8: note = HCDR1
GFTFSNVW

SEQ ID NO: 39 - protein, 8 aa, synthetic construct
  source 1..8: note = HCDR1
GFTFSNVM

SEQ ID NO: 40 - protein, 8 aa, synthetic construct
  source 1..8: note = HCDR1
GFIFSKAW

SEQ ID NO: 41 - protein, 8 aa, synthetic construct
  source 1..8: note = HCDR1
GFTFITYS

SEQ ID NO: 42 - protein, 8 aa, synthetic construct
  source 1..8: note = HCDR1
GGSISSYF

SEQ ID NO: 43 - protein, 8 aa, synthetic construct
  source 1..8: note = HCDR1
GGSVFSYF

SEQ ID NO: 44 - protein, 8 aa, synthetic construct
  source 1..8: note = HCDR1
GYSFTSYW

SEQ ID NO: 45 - protein, 8 aa, synthetic construct
  source 1..8: note = HCDR2
LNPNSGGT

SEQ ID NO: 46 - protein, 8 aa, synthetic construct
  source 1..8: note = HCDR2
INPYSGGT

SEQ ID NO: 47 - protein, 8 aa, synthetic construct
  source 1..8: note = HCDR2
INPNSGGT

SEQ ID NO: 48 - protein, 8 aa, synthetic construct
  source 1..8: note = HCDR2
INPSVGST

SEQ ID NO: 49 - protein, 8 aa, synthetic construct
  source 1..8: note = HCDR2
IIPISGTE

SEQ ID NO: 50 - protein, 8 aa, synthetic construct
  source 1..8: note = HCDR2
IIPIFGTA

SEQ ID NO: 51 - protein, 8 aa, synthetic construct
  source 1..8: note = HCDR2
IIPIFGTE

SEQ ID NO: 52 - protein, 8 aa, synthetic construct
  source 1..8: note = HCDR2
FSPIFGTA

SEQ ID NO: 53 - protein, 10 aa, synthetic construct
  source 1..10: note = HCDR2
IKSKTDGGTI

SEQ ID NO: 54 - protein, 10 aa, synthetic construct
  source 1..10: note = HCDR2
IKSKADGGTI

SEQ ID NO: 55 - protein, 10 aa, synthetic construct
  source 1..10: note = HCDR2
IKSKTDGGTT

SEQ ID NO: 56 - protein, 8 aa, synthetic construct
  source 1..8: note = HCDR2
ISSISSYI

SEQ ID NO: 57 - protein, 7 aa, synthetic construct
  source 1..7: note = HCDR2
GHYSGNS

SEQ ID NO: 58 - protein, 7 aa, synthetic construct
  source 1..7: note = HCDR2
IDHSGGT

SEQ ID NO: 59 - protein, 8 aa, synthetic construct
  source 1..8: note = HCDR2
IYPGNSDT

SEQ ID NO: 60 - protein, 8 aa, synthetic construct
  source 1..8: note = HCDR2
IWPGDSDT

SEQ ID NO: 61 - protein, 11 aa, synthetic construct
  source 1..11: note = HCDR3
ARTLYYYGMDV

SEQ ID NO: 62 - protein, 11 aa, synthetic construct
  source 1..11: note = HCDR3
ARSYYYYGMDV

SEQ ID NO: 63 - protein, 15 aa, synthetic construct
  source 1..15: note = HCDR3
AVDSSGSRYYYGMDV

SEQ ID NO: 64 - protein, 12 aa, synthetic construct
  source 1..12: note = HCDR3
ARNGATSDAFDI

SEQ ID NO: 65 - protein, 12 aa, synthetic construct
  source 1..12: note = HCDR3
ARNGATSDAFDM

SEQ ID NO: 66 - protein, 12 aa, synthetic construct
  source 1..12: note = HCDR3
AWGSGNFNWFDP

SEQ ID NO: 67 - protein, 10 aa, synthetic construct
  source 1..10: note = HCDR3
TTGELLGFDY

SEQ ID NO: 68 - protein, 9 aa, synthetic construct
  source 1..9: note = HCDR3
TTGGYVFDY

SEQ ID NO: 69 - protein, 14 aa, synthetic construct
  source 1..14: note = HCDR3
AREGIVGPTGYFDS

SEQ ID NO: 70 - protein, 11 aa, synthetic construct
  source 1..11: note = HCDR3
AREGLHDAFDI

SEQ ID NO: 71 - protein, 10 aa, synthetic construct
  source 1..10: note = HCDR3
ARGGSYYLDY

SEQ ID NO: 72 - protein, 11 aa, synthetic construct
  source 1..11: note = HCDR3
ARSSGGTAMDV

SEQ ID NO: 73 - protein, 12 aa, synthetic construct
  source 1..12: note = LCDR1
QSVLINSNNQNY

SEQ ID NO: 74 - protein, 12 aa, synthetic construct
  source 1..12: note = LCDR1
QNIINSSTNKNY

SEQ ID NO: 75 - protein, 12 aa, synthetic construct
  source 1..12: note = LCDR1
QSVLYSSNNTSY

SEQ ID NO: 76 - protein, 6 aa, synthetic construct
  source 1..6: note = LCDR1
QSVRSN

SEQ ID NO: 77 - protein, 7 aa, synthetic construct
  source 1..7: note = LCDR1
LSVSSNY

SEQ ID NO: 78 - protein, 7 aa, synthetic construct
  source 1..7: note = LCDR1
QSVSSNY

SEQ ID NO: 79 - protein, 6 aa, synthetic construct
  source 1..6: note = LCDR1
QGISSY

SEQ ID NO: 80 - protein, 12 aa, synthetic construct
  source 1..12: note = LCDR1
QSLLHNNGFNCL

SEQ ID NO: 81 - protein, 12 aa, synthetic construct
  source 1..12: note = LCDR1
QSLLHSNGFNCL

SEQ ID NO: 82 - protein, 8 aa, synthetic construct
  source 1..8: note = LCDR1
NSNIGSYI

SEQ ID NO: 83 - protein, 8 aa, synthetic construct
  source 1..8: note = LCDR1
SSNIGNHY

SEQ ID NO: 84 - protein, 8 aa, synthetic construct
  source 1..8: note = LCDR1
ISNIGSNT

SEQ ID NO: 85 - protein, 8 aa, synthetic construct
  source 1..8: note = LCDR1
SSNIGSNT

SEQ ID NO: 86 - protein, 11 aa, synthetic construct
  source 1..11: note = LCDR1
QSLLHSNGYNT

SEQ ID NO: 87-97 - skipped (11 entries)

SEQ ID NO: 98 - protein, 9 aa, synthetic construct
  source 1..9: note = LCDR3
QQYYNSPFT

SEQ ID NO: 99 - protein, 9 aa, synthetic construct
  source 1..9: note = LCDR3
QQYYSTPFT

SEQ ID NO: 100 - protein, 9 aa, synthetic construct
  source 1..9: note = LCDR3
QQYYITPFT

SEQ ID NO: 101 - protein, 9 aa, synthetic construct
  source 1..9: note = LCDR3
QQYNYWPYT

SEQ ID NO: 102 - protein, 9 aa, synthetic construct
  source 1..9: note = LCDR3
QQYGSSPLT

SEQ ID NO: 103 - protein, 9 aa, synthetic construct
  source 1..9: note = LCDR3
QQYYSYPLT

SEQ ID NO: 104 - protein, 9 aa, synthetic construct
  source 1..9: note = LCDR3
MQTLQTPIT

SEQ ID NO: 105 - protein, 11 aa, synthetic construct
  source 1..11: note = LCDR3
AAWDDSLNGVV

SEQ ID NO: 106 - protein, 12 aa, synthetic construct
  source 1..12: note = LCDR3
GTWDSRLSAYWV

SEQ ID NO: 107 - protein, 11 aa, synthetic construct
  source 1..11: note = LCDR3
AAWDDSLNGRV

SEQ ID NO: 108 - protein, 9 aa, synthetic construct
  source 1..9: note = LCDR3
MQGLQTQWT

SEQ ID NO: 109 - protein, 449 aa, synthetic construct
  source 1..449: note = STLX 2012 HC DLE
QVQLLQSGAEVKKPGSSVKVSCKASGGTFSIYAIDWVRQAPGQGLEWMGGIIPIFGTANSAQKFQGRVTITADKSTSTAYMELSSLRSEDTAVYYCARNGATSDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 215 aa, synthetic construct
  source 1..215: note = STLX 2012 LC
EIVLTQSPGTLSLSPGERATLSCRASLSVSSNYLAWFQQRPGQAPRLLIHGVSRRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0392013 A1
(43) Pub. Date: Nov. 28, 2024

(54) IL1RAP ANTIBODIES AND USES THEREOF

(71) Applicant: STELEXIS THERAPEUTICS, LLC, New York, NY (US)

(72) Inventors: Patrick James DOYLE, New York, NY (US); Zoi KAROULIA, New York, NY (US); Carmine CARPENITO, Hartsdale, NY (US); Jiahao CHEN, New York, NY (US); Lumie Marie Josephine BENARD, Yonkers, NY (US); Adriana Permaul ROOPNARIANE, Plainview, NY (US); Yasumi NAKAYAMA, Astoria, NY (US); Lynn BIDERMAN, New York, NY (US); Bozena BUGAJ-GAWEDA, Westwood, NJ (US); Ilhem GUERNAH, Sunnyside, NY (US); Ivo C. LORENZ, New York, NY (US); Dana Yen Mei DUEY, San Diego, CA (US); John Andrew LIPPINCOTT, San Diego, CA (US)

(73) Assignee: STELEXIS THERAPEUTICS, LLC, New York, NY (US)

(21) Appl. No.: 18/290,778

(22) PCT Filed: Sep. 27, 2022
(86) PCT No.: PCT/US2022/037530

§ 371 Date: Jan. 21, 2024

Related U.S. Application Data
(60) Provisional application No. 63/223,994, filed on Jul. 21, 2021.

(51) Int. Cl. C07K 16/28; A61K 39/00; A61P 35/02

(52) U.S. Cl. CPC C07K 16/2866; A61P 35/02; A61K 2039/505; C07K 2317/565; C07K 2317/72; C07K 2317/732; C07K 2317/76; C07K 2317/92

(57) ABSTRACT

The present disclosure provides isolated anti-IL1RAP antibodies comprising the complementarity determining regions (CDRs) amino acid sequences, as disclosed herein. The disclosure further provides specific variable heavy chain and variable light chain amino acid sequences of these isolated anti-IL1RAP antibodies. The anti-IL1RAP antibodies target downstream IL1RAP activity appear useful for treating diseases such as cancer.

### FIELD OF INVENTION

The present disclosure relates in general to the field of antibody technology. In one embodiment, the present disclosure provides anti-IL1RAP antibodies and uses of the same.

### CROSS REFERENCE TO RELATED APPLICATIONS

This application claims the benefit of U.S. Provisional Application Ser. No. 63/223,994, filed on Jul. 21, 2021, which is incorporated in its entirety herein by reference.

### BACKGROUND

The human Interleukin-1 receptor accessory protein, also known as IL1RAP, IL-1RAcP, and IL1R3 is a protein encoded by the IL1RAP gene. Upon stimulation by IL-1α or IL-1β cytokine, IL1RAP interacts and forms a heteromeric receptor complex with the Interleukin 1 Receptor (IL1R1). The functional IL1R1/IL1/IL1RAP complex initiates the transmission of IL-1 signaling pathway that induces the synthesis of acute phase and proinflammatory proteins through activation of NFκB.

IL1RAP also interacts and forms heteromeric complex with the Interleukin 1 Receptor-like 1, also known as IL1RL1 and ST2 upon stimulation by another member of the IL-1 family of cytokines, IL-33. The functional IL1RL1/IL33/IL1RAP complex activates NFκB and MAP kinase signaling pathways to enhance mast cell, TH2, regulatory T cell (Treg) and innate lymphoid cell type 2 functions.

Additionally, IL1RAP interacts and forms heteromeric complex with the Interleukin-1 Receptor-like 2, also known as IL1RL2, IL-1Rrp2 and IL36R upon stimulation by IL36 cytokine. The functional IL36R/IL36/IL1RAP complex activates NFκB and MAP kinases to induce various inflammatory and skin diseases.

IL1RAP has been identified to be overexpressed in AML hematopoietic stem and progenitor cells in multiple genetic subtypes of AML and in high-risk myelodysplastic syndromes (MDS) and IL-1 has been shown to be upregulated in several types of cancer, including pancreatic, head and neck, lung, breast, colon, and melanomas.

Given the link between inflammation and human disease, IL-1 has been associated with a critical role in the pathogenesis of several rheumatic diseases, as well as cancer initiation and progression, while patients with high levels of IL-1 are related to poor prognosis. IL33 has been also associated with disease including acute myocardial infarction, asthma, and eosinophilic pneumonia and has been characterized is recent studies as a key driver of treatment resistance in cancer. IL36 has a significant role in the pathogenesis of skin diseases, including psoriasis and has been linked to psoriatic arthritis, systemic lupus, inflammatory bowel disease, ulcerative colitis, Crohn's disease, and Sjogren's syndrome and neoplastic disorders.

NFκB, which is activated upon stimulation of all the above pathways where IL1RAP is involved, regulates the expression of several genes that are important in DNA transcription and cell survival, is involved in cellular responses to stimuli, such as stress, and plays a key role in regulating immune responses to infection. Impaired function of NFκB, which has been characterized as first responder to harmful cellular stimuli, has been linked to inflammatory and autoimmune diseases and cancer.

Anakinra (Kineret®, Swedish Orphan Biovitrum; Sweden) is the recombinant version of IL1Ra (IL-1 receptor antagonist) that blocks binding of IL-1 to IL1R1 and has been approved for the treatment of Cryopirin-Associated Periodic Syndromes including Neonatal-Onset Multisystem Inflammatory disease, Deficiency of Interleukin-1 Receptor Antagonist (DIRA), and rheumatoid arthritis. Canakinumab (Ilaris®, Novartis; Switzerland) is a monoclonal antibody that targets IL1-β and has also been indicated for the treatment of auto-inflammatory Cryopyrin-Associated Syndromes, as well as 3 rare autoimmune diseases, the Tumor Necrosis Factor Receptor Associated Periodic Syndrome (TRAPS), the Hyperimmunoglobulin D Syndrome (HIDS)/Mevalonate kinase deficiency (MKD), and the Familial Mediterranean Fever (FMF). Canakinumab is now being evaluated in clinical trials for the treatment of NSCLC. Rilonacept (Arcalyst™, Regeneron; NY, USA) is a dimeric fusion decoy receptor consisted of the extracellular domains of IL1R1 and IL1RAP linked to the Fc region of human IgG1 that neutralizes IL-1 and is indicated for the treatment of Recurrent Pericarditis (RP) and Cryopyrin-Associated Periodic Syndromes (CAPS), including Familial Cold Autoinflammatory Syndrome (FCAS), Muckle-Wells Syndrome (MWS), and Deficiency of Interleukin-1 Receptor Antagonist (DIRA). Therapeutic approaches including IL1RAP antibodies are now being evaluated in clinical trials for the treatment of cancer.

All the above indicate an important role of IL1RAP in disease. Thus, there remains an unmet need for developing the tools for treating diseases and conditions associated with IL1RAP, and methods of use thereof.

Described herein are human IL1RAP antibodies and uses thereof, wherein the antibodies target downstream IL1RAP activity and disease development. Specifically, the human IL1RAP antibodies developed herein exhibit high affinity binding to IL1RAP that blocks NFκB activity, inhibits downstream oncogenic signaling, and cancer cell proliferation and differentiation.

### SUMMARY

In one embodiment, the present disclosure provides isolated anti-IL1RAP antibodies comprising three complementarity determining regions (CDRs) on a heavy chain (HCDR1, HCDR2, and HCDR3) and three CDRs on a light chain (LCDR1, LCDR2, and LCDR3), wherein the sequences for the CDRs are disclosed herein.

In another embodiment, the present disclosure provides isolated anti-IL1RAP antibodies, each of which comprising a heavy chain variable region and a light chain variable region as disclosed herein.

The present disclosure also provides compositions comprising the anti-IL1RAP antibodies disclosed herein.

In another embodiment, the present disclosure provides isolated polynucleotide sequences encoding the anti-IL1RAP antibodies disclosed herein; vectors comprising the polynucleotide sequences; and host cells comprising the vectors.

In another embodiment, the present disclosure further provides methods of treating a disease in a subject, comprising the step of administering to the subject a composition comprising an effective amount of the anti-IL1RAP antibodies disclosed herein. In one embodiment, the disease can be a hematological cancer or a solid tumor.

### BRIEF DESCRIPTION OF THE DRAWINGS

The subject matter disclosed herein describing IL1RAP antibodies and uses thereof is particularly pointed out and distinctly claimed in the concluding portion of the specification. These antibodies and uses thereof, however, both as to organization and method of operation, together with objects, features, and advantages thereof, may best be understood by reference to the following detailed description when read with the accompanying drawings in which:

FIG. 1. Binding of mAbs to human IL1RAP. Binding of mAbs to human IL1RAP protein was determined by cell-free ELISA. The representative graph presented, shows that mAbs bound to human IL1RAP in a dose dependent manner for two independent IL1RAP mAb clones, STLX2012 and STLX2043.

FIG. 2. Size-exclusion analysis of IL1RAP mAbs. Antibodies were profiled by size-exclusion analysis conducted by SEC-HPLC. A representative graph for clone STLX2043 is presented. Similar analysis was performed for all 18 antibodies listed in Table 1.

FIG. 3. Expression analysis of IL1RAP mAbs. Gel profiles showing the expression and characteristics of a representative mAb (STLX2043) under non-reducing and reducing conditions is shown. Molecular weight standards are shown to the left of each gel. Similar analysis was performed for all 18 antibodies listed in Table 1.

FIG. 4. Blocking of IL1R1/IL1p/IL1RAP complex formation by IL1RAP mAbs. Representative curves for mAbs STLX2012 and STLX2043 show that mAbs blocked binding of IL1R1/IL1β complex to IL1RAP in a dose dependent manner.

FIGS. 5A and 5B. Inhibition of IL1-induced NFκB activity by IL1RAP mAbs. Representative panels of IL1RAP mAbs show that all of mAbs inhibited the IL1-induced NFκB activity in a dose-dependent manner. Inhibition was measured by the HEK-BlueIL1β cell-based reporter assay. #1 and #2 show different antibodies in the same assay.

FIGS. 6A and 6B. Inhibition of IL33-induced NFκB activity by IL1RAP mAbs. Representative panels of IL1RAP mAbs, show that all of the mAbs inhibited the IL33-induced NFκB activity in a dose-dependent manner. Inhibition was measured by the HEKBlue-IL33 cell-based reporter assay. #1 and #2 show different antibodies in the same assay.

FIGS. 7A and 7B. Inhibition of IL1 signaling in acute myeloid leukemia (AML) patient samples by IL1RAP mAbs. mAbs inhibited IL1-induced downstream signaling which was monitored by Western blot using phospho-specific antibodies against ERK and NFκB. (FIG. 7A) The results of a representative mAb (STLX2012) are shown. Actin was used as a loading control. The bar graphs presented in FIG. 7B present % inhibition of phosphorylation of ERK and NFκB. The results presented are for AML patient 3. Controls: IL1β only, and Isotype+IL1β (Isotype is IgG1 control that has the same backbone as the IL1RAP antibodies but does not target IL1RAP).

FIGS. 8A and 8B. Inhibition of IL33 signaling in AML patient samples by IL1RAP mAbs. mAbs inhibited IL33-induced downstream signaling which was monitored by Western blot using phospho-specific antibodies against ERK, p38 and NFκB (FIG. 8A). Representative results of mAb STLX2012 are shown here. Actin was used as a loading control. The bar graphs of FIG. 8B present % inhibition of phosphorylation of ERK, p38 and NFκB. The results presented are for AML patient 1. Controls: IL-33 only, and Isotype+IL33 (Isotype is IgG1 control that has the same backbone as the IL1RAP antibodies but does not target IL1RAP).

FIGS. 9A and 9B. Inhibition of IL1 signaling in AML cells by IL1RAP mAbs. Anti-IL1RAP mAb (STLX2012) inhibited IL1-induced downstream signaling which was monitored by Western blot using phospho-specific antibodies against p38 and NFκB (FIG. 9A). Actin was used as a loading control. The bar graphs presented in FIG. 9B, show % inhibition of phosphorylation of p38 and NFκB. Controls: IL1β only, and Isotype+IL1β (Isotype is IgG1 control that has the same backbone as the IL1RAP antibodies but does not target IL1RAP). This is representative data for one experiment. N=3 (data not shown)

FIGS. 10A and 10B. Inhibition of IL1 signaling in chronic myelogenous leukemia (CML) K562 cells by IL1RAP mAbs. mAbs inhibited I-induced downstream signaling which was monitored by Western blot using phospho-specific antibody against NFκB. Representative results are shown in FIG. 10A for mAb STLX2012. Actin was used as a loading control. The bar graphs in FIG. 10B present % inhibition of phosphorylation of NFκB. Controls: IL1β only, and Isotype+IL1β (Isotype is IgG1 control that has the same backbone as the IL1RAP antibodies but does not target IL1RAP). This is representative data for one experiment. N=3 (data not shown)

FIGS. 11A-11L. Inhibition of IL1 signaling in solid tumor cancer cells by IL1RAP mAbs. Pancreatic cancer cell (A6L) (FIGS. 11A and 11B), head and neck squamous cell carcinoma (HNSCC) cells (CAL33) (FIGS. 11C and 11D), bladder cancer cells (5637) (FIGS. 11E and 11F), non-small cell lung cancer (NSCLC) cells (A549) (FIGS. 11G and 11H), colorectal cancer cells (Colo205) (FIGS. 11I and 11J), and triple negative breast cancer (TNBC) cells (HCC70) (FIGS. 11K and 11L). mAbs inhibited IL1-induced downstream signaling which was monitored by Western blot using phospho-specific antibody against ERK, AKT, p38 and NFκB (FIGS. 11A, 11C, 11E, 11G, 11I, and 11K). Actin was used as a loading control. The bar graphs of FIGS. 11B, 11D, 11F, 11H, 11J, and 11L present % inhibition of phosphorylation of ERK, AKT, p38 and NFκB. An example of one cell line is shown for each cancer type. Representative results are shown for mAbs STLX2012 and STLX2045.

FIGS. 12A-12F. Inhibition of IL36 signaling in solid tumor cancer cells by IL1RAP mAbs. Pancreatic cancer cells (HPAC) (FIGS. 12A-12B), HNSCC cells (CAL33) (FIGS. 12C-12D) and bladder cancer cells (5637) (FIGS. 12E-12F). mAbs inhibited IL36-induced downstream signaling, which was monitored by Western blot using phospho-specific antibody against ERK, AKT, p38 and NFκB (FIGS. 12A, 12C, and 12E). Actin was used as a loading control. The bar graphs of FIGS. 12B, 12D, and 12F present % inhibition of phosphorylation of ERK, AKT, p38 and NFκB. An example of one cell line is shown for each cancer type. Representative results are shown for mAbs STLX2012 and STLX2045.

FIG. 13. Inhibition of proliferation of AML patient samples by IL1RAP mAbs. mAbs inhibited cell proliferation, which was measured by Cell-Titer Glo. An example of one AML patient sample is shown using representative mAb STLX2012.

FIG. 14. Inhibition of proliferation of AML cells (THP-1) by IL1RAP mAbs. mAbs inhibited cell proliferation, which was measured by Cell-Titer Glo using representative mAb STLX2012.

FIG. 15. Inhibition of proliferation of CML cells by IL1RAP mAbs. mAbs inhibited cell proliferation, which was measured by Cell-Titer Glo using representative mAb STLX2012.

FIG. 16. Inhibition of viability of patient-derived AML samples by IL1RAP mAbs. mAbs inhibited cell proliferation, which was measured by Cell-Titer Glo using representative mAbs STLX2005, STLX2012, and STLX2027. Similar results were observed for STLX2045 (Data not shown).

FIG. 17. Inhibition of clonogenic capacity of AML patient samples by IL1RAP mAbs. mAbs inhibited the clonogenic capacity, which was calculated by counting the formation of colonies. An example of one AML patient sample is shown using representative mAb STLX2012. Similar results were observed for other mAbs (Data not shown).

FIG. 18. IL1RAP mAbs do not inhibit the clonogenic capacity of healthy control samples. mAbs did not affect the clonogenic capacity of healthy control samples, which was calculated by counting the formation of colonies. An example of one healthy control sample is shown using representative mAb STLX2012. Similar results were observed for other mAbs (Data not shown).

FIGS. 19A and 19B. Induction of expression of differentiation markers by IL1RAP mAbs. mAbs induced the expression of CD14 (FIG. 19A) and CD15 (FIG. 19B) differentiation markers, which was monitored by flow cytometry using representative mAb STLX2012.

FIG. 20. Blocking of IL6 secretion by IL1RAP mAbs. mAbs blocked the IL1-induced secretion of IL6, which was monitored by ELISA using representative mAbs STLX2012 and STLX2043. Isotype was used as control.

FIG. 21. Blocking of IL8 secretion by IL1RAP mAbs. mAbs blocked the IL36-induced secretion of IL8, which was monitored by ELISA using representative mAbs STLX2012 and STLX2043. Isotype was used as a negative control.

FIG. 22. Blocking of IL36γ signally by IL1RAP mAb in a dose dependent manner. Representative STLX2012 mAb inhibited IL36γ-induced signaling in a dose-dependent manner. Solid small circle represents STLX2012 and open box represents control non-specific IgG1.

FIGS. 23A-23F. STLX2012 induces ADCC reporter in multiple cell lines and patient-derived AML cells. ADCC (NFκB) activity was measured by the Jurkat-Lucia NFAT-CD16 reporter assay. FIG. 23A THP-1 AML cell line. FIG. 23B SK-Mel-5 melanoma cell line. FIGS. 23C-23F AML patient sample. Solid small circle represents STLX2012 and open box represents control non-specific IgG1.

FIGS. 24A and 24B. Amino acid sequence and structure of STLX2012-DLE antibody. FIG. 24A presents the full length of the heavy chain (HC) amino acid sequence of the STLX2012 antibody, wherein the HC comprises substitution mutations S239D, A330L, and 1332E (DLE mutations) in the Fc region (STLX2012 HC DLE; SEQ ID NO: 109). The mutated amino acids are indicated as a bold, italicized amino acid that is underlined. The light chain (LC) amino acid sequence of the STLX 2012-DLE antibody is presented in SEQ ID NO: 110. No amino acid changes were made to the LC. FIG. 24B presents a cartoon schematic of the STLX2012-DLE antibody indicating the location within the HC of the 3 mutated amino acids.

FIG. 25. DLE mutations in STLX2012 enhances ADCC-mediated reporter cell activity. Addition of substitution mutations S239D, A330L, and 1332E (DLE mutations) in the Fc region of mAb STLX2012 enhances the fold-induction of ADCC-mediated reporter cell activity compared with STLX2012 lacking these mutations. Solid small circle represents STLX2012, solid box is STLX2012-DLE, open box represents control non-specific IgG1, and open triangle is a control non-specific IgG1 having the DLE mutations.

FIGS. 26A-26C. STLX2012 inhibited colony formation capacity. Bar graphs show mAb STLX2012 inhibited colony formation in three different AML patient (Pt) derived cell samples, AML Pt 1 (FIG. 26A), AML Pt 35 (FIG. 26B), and AML Pt 9 (FIG. 26C). IgG—control non-specific IgG antibody, +AZA includes 100 nM Azacitidine, +Ven includes 100 nM Venetoclax.

FIGS. 27A and 27B. STLX2012 antibody inhibited AML patient sample engraftment in immunodeficient mice. Graphs show percent (%) human CD45+AML cells in the bone marrow (FIG. 27A) and spleen (FIG. 27B) following treatment with control non-specific IgG1 antibody and different doses of mAb STLX2012 (1 milligrams per kilogram of body weight (mpk), 10 mpk, and 30 mpk).

### DETAILED DESCRIPTION

In the following detailed description, numerous specific details are set forth in order to provide a thorough understanding of the anti-IL1RAP antibodies described and exemplified herein, and therapeutic uses thereof. However, it will be understood by those skilled in the art that production and use of the anti-IL1RAP antibodies may in certain cases, be practiced without these specific details. In other instances, well-known methods, procedures, and components have not been described in detail so as not to obscure the disclosure presented herein.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind an IL1RAP epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

Half maximal effective concentration (EC₅₀) refers to the concentration of a drug, antibody or toxicant which induces a response halfway between the baseline and maximum responses after a specified exposure time. In some embodiments, the response comprises a binding affinity. A skilled artisan would appreciate that as used herein in certain embodiments, the EC₅₀ measurement of an anti-IL1RAP antibody disclosed herein provides a measure of a half-maximal binding of the anti-IL1RAP antibody to the IL1RAP antigen (EC₅₀ binding).

In some embodiments, EC₅₀ comprises the concentration of antibody required to obtain a 50% agonist response that would be observed upon antibody binding. In certain embodiments, a measure of EC₅₀ is commonly used as a measure of a drug's potency and may in some embodiments, reflect the binding of the antibody to the receptor. In some embodiments, anti-IL1RAP antibodies having nanomolar EC₅₀ binding concentration measurements comprise tight binding anti-IL1RAP antibodies. In certain embodiments, an anti-IL1RAP antibody disclosed herein comprises a tight binder to the IL1RAP molecule.

In some embodiments, the binding EC₅₀ of an anti-IL1RAP antibody is in the nanomolar range. In some embodiments, the binding EC₅₀ of an anti-IL1RAP antibody comprises a range of about 0.05-100 nM. In some embodiments, the binding EC₅₀ of an anti-IL1RAP antibody comprises a range of about 0.05-50 nM. In some embodiments, the binding EC₅₀ of an anti-IL1RAP antibody comprises a range of about 0.05-20 nM. In some embodiments, the binding EC₅₀ of an anti-IL1RAP antibody comprises a range of about 0.05-10 nM. In some embodiments, the binding EC₅₀ of an anti-IL1RAP antibody comprises a range of about 0.1-100 nM. In some embodiments, the binding EC₅₀ of an anti-IL1RAP antibody comprises a range of about 0.1-50 nM. In some embodiments, the binding EC₅₀ of an anti-IL1RAP antibody comprises a range of about 0.1-20 nM. In some embodiments, the binding EC₅₀ of an anti-IL1RAP antibody comprises a range of about 0.1-10 nM. In some embodiments, the binding EC₅₀ of an anti-IL1RAP antibody comprises a range of about 1-100 nM. In some embodiments, the binding EC₅₀ of an anti-IL1RAP antibody comprises a range of about 1-20 nM. In some embodiments, the binding EC₅₀ of an anti-IL1RAP antibody comprises a range of about 20-40 nM. In some embodiments, the binding EC₅₀ of an anti-IL1RAP antibody comprises a range of about 40-60 nM. In some embodiments, the binding EC₅₀ of an anti-IL1RAP antibody comprises a range of about 60-80 nM. In some embodiments, the binding EC₅₀ of an anti-IL1RAP antibody comprises a range of about 80-100 nM. In some embodiments, the binding EC₅₀ of an anti-IL1RAP antibody comprises a range of about 1-40 nM. In some embodiments, the binding EC₅₀ of an anti-IL1RAP antibody comprises a range of about 1-60 nM. In some embodiments, the binding EC₅₀ of an anti-IL1RAP antibody comprises a range of about 1-80 nM. In some embodiments, the binding EC₅₀ of an anti-IL1RAP antibody comprises a range of about 1-50 nM. In some embodiments, the binding EC₅₀ of an anti-IL1RAP antibody comprises a range of about 0.05-5 nM. In some embodiments, the binding EC₅₀ of an anti-IL1RAP antibody comprises a range of about 0.1-5 nM. In some embodiments, the binding EC₅₀ of an anti-IL1RAP antibody comprises a range of about 0.05-20 nM.

In some embodiments, the binding EC₅₀ of an anti-IL1RAP antibody comprises a range of about 0.05-5 nM. In some embodiments, the binding EC₅₀ of an anti-IL1RAP antibody comprises a range of about 0.1-5 nM. In some embodiments, the binding EC₅₀ of an anti-IL1RAP antibody comprises a range of about 1-5 nM. In some embodiments, the binding EC₅₀ of an anti-IL1RAP antibody comprises a range of about 0.05-10 nM. In some embodiments, the binding EC₅₀ of an anti-IL1RAP antibody comprises a range of about 0.1-10 nM. In some embodiments, the binding EC₅₀ of an anti-IL1RAP antibody comprises a range of about 1-10 nM. In some embodiments, the binding EC₅₀ of an anti-IL1RAP antibody comprises a range of about 5-10 nM. In some embodiments, the binding EC₅₀ of an anti-IL1RAP antibody comprises a range of about 0.05-15 nM. In some embodiments, the binding EC₅₀ of an anti-IL1RAP antibody comprises a range of about 0.01-15 nM. In some embodiments, the binding EC₅₀ of an anti-IL1RAP antibody comprises a range of about 1-15 nM.

As used herein, the term “antibody” encompasses an antibody fragment or fragments that retain binding specificity including, but not limited to, IgG, heavy chain variable region (VH), light chain variable region (VL), Fab fragments, F(ab′)₂ fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide. The linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

In one embodiment, the anti-IL1RAP antibodies disclosed herein can be incorporated as part of a bispecific antibody. In one embodiment, the anti-IL1RAP antibodies disclosed herein can be incorporated as part of a multi-specific antibody. As it is generally known in the art, a bispecific antibody is a recombinant protein that includes antigen-binding fragments of two different monoclonal antibodies, and is thereby capable of binding two different antigens. In one embodiment, the anti-IL1RAP antibodies disclosed herein can be incorporated as part of a multi-specific antibody. A multi-specific antibody is a recombinant protein that includes antigen-binding fragments of at least two different monoclonal antibodies, such as two, three or four different monoclonal antibodies.

In some embodiments, the anti-IL1RAP antibodies disclosed herein are bi-valent for IL1RAP. In some embodiments, the anti-IL1RAP antibodies disclosed herein are monovalent for binding IL1RAP.

In some embodiments, bispecific, tri-specific, or multi-specific antibodies are used for cancer immunotherapy by simultaneously targeting more than one antigen target, for example but not limited to, a cytotoxic T cell (CTL) as well as a tumor associated antigen (TAA), or simultaneously targeting more than one CTL, such as targeting a CTL receptor component such as CD3, an effector natural killer (NK) cells, and a tumor associated antigen (TAA).

Provided herein are embodiments of human monoclonal antibodies that specifically bind to the Interleukin-1 Receptor Accessory Protein (IL1RAP). Exemplification demonstrates that the antibodies block IL1R1/IL1/IL1RAP complex formation and suppress IL1 and IL33 induced NFκB activity. These antibodies also inhibit signaling and proliferation of cells from AML patients' samples, leukemia cell lines, and solid tumor cancer cell lines. Further the IL1RAP antibodies suppress the clonogenic capacity of AML patients' samples. The monoclonal IL1RAP antibodies can be used for the treatment of IL1RAP mediated diseases, which include but are not limited to cancers including AML, CML, and pancreatic, bladder, NSCLC, TNBC and HNSCC cancers.

### Anti-IL1RAP Antibodies

The present disclosure provides a number of anti-IL1RAP antibodies. In one embodiment, each of the anti-IL1RAP antibodies comprises a set of three complementarity determining regions (CDRs) on a heavy chain (HCDR1, HCDR2, and HCDR3) and a set of three CDRs on a light chain (LCDR1, LCDR2, and LCDR3).

In one embodiment, the HCDR1, HCDR2, and HCDR3 comprises the amino acid sequences of SEQ ID NOs:33, 45 and 61, and the LCDR1, LCDR2, and LCDR3 comprises the amino acid sequences of SEQ ID NOs:73, 87 and 98.

In one embodiment, the HCDR1, HCDR2, and HCDR3 comprises the amino acid sequences of SEQ ID NOs:33, 46 and 62, and the LCDR1, LCDR2, and LCDR3 comprises the amino acid sequences of SEQ ID NOs:74, 87 and 99.

In one embodiment, the HCDR1, HCDR2, and HCDR3 comprises the amino acid sequences of SEQ ID NOs:33, 47 and 62, and the LCDR1, LCDR2, and LCDR3 comprises the amino acid sequences of SEQ ID NOs:75, 88 and 100.

In one embodiment, the HCDR1, HCDR2, and HCDR3 comprises the amino acid sequences of SEQ ID NOs:34, 48 and 63, and the LCDR1, LCDR2, and LCDR3 comprises the amino acid sequences of SEQ ID NOs:76, 89 and 101.

In one embodiment, the HCDR1, HCDR2, and HCDR3 comprises the amino acid sequences of SEQ ID NOs:35, 49 and 64, and the LCDR1, LCDR2, and LCDR3 comprises the amino acid sequences of SEQ ID NOs:77, 90 and 102.

In one embodiment, the HCDR1, HCDR2, and HCDR3 comprises the amino acid sequences of SEQ ID NOs:35, 50 and 65, and the LCDR1, LCDR2, and LCDR3 comprises the amino acid sequences of SEQ ID NOs:77, 90 and 102.

In one embodiment, the HCDR1, HCDR2, and HCDR3 comprises the amino acid sequences of SEQ ID NOs:35, 51 and 64, and the LCDR1, LCDR2, and LCDR3 comprises the amino acid sequences of SEQ ID NOs:77, 90 and 102.

In one embodiment, the HCDR1, HCDR2, and HCDR3 comprises the amino acid sequences of SEQ ID NOs:36, 50 and 64, and the LCDR1, LCDR2, and LCDR3 comprises the amino acid sequences of SEQ ID NOs:78, 90 and 102.

In one embodiment, the HCDR1, HCDR2, and HCDR3 comprises the amino acid sequences of SEQ ID NOs:37, 52 and 66, and the LCDR1, LCDR2, and LCDR3 comprises the amino acid sequences of SEQ ID NOs:79, 91 and 103.

In one embodiment, the HCDR1, HCDR2, and HCDR3 comprises the amino acid sequences of SEQ ID NOs:38, 53 and 67, and the LCDR1, LCDR2, and LCDR3 comprises the amino acid sequences of SEQ ID NOs:80, 92 and 104.

In one embodiment, the HCDR1, HCDR2, and HCDR3 comprises the amino acid sequences of SEQ ID NOs:39, 54 and 67, and the LCDR1, LCDR2, and LCDR3 comprises the amino acid sequences of SEQ ID NOs:81, 92 and 104.

In one embodiment, the HCDR1, HCDR2, and HCDR3 comprises the amino acid sequences of SEQ ID NOs:40, 55 and 68, and the LCDR1, LCDR2, and LCDR3 comprises the amino acid sequences of SEQ ID NOs:82, 93 and 105.

In one embodiment, the HCDR1, HCDR2, and HCDR3 comprises the amino acid sequences of SEQ ID NOs:41, 56 and 69, and the LCDR1, LCDR2, and LCDR3 comprises the amino acid sequences of SEQ ID NOs:83, 94 and 106.

In one embodiment, the HCDR1, HCDR2, and HCDR3 comprises the amino acid sequences of SEQ ID NOs:42, 57 and 70, and the LCDR1, LCDR2, and LCDR3 comprises the amino acid sequences of SEQ ID NOs:84, 95 and 107.

In one embodiment, the HCDR1, HCDR2, and HCDR3 comprises the amino acid sequences of SEQ ID NOs:43, 58 and 70, and the LCDR1, LCDR2, and LCDR3 comprises the amino acid sequences of SEQ ID NOs:85, 96 and 107.

In one embodiment, the HCDR1, HCDR2, and HCDR3 comprises the amino acid sequences of SEQ ID NOs:44, 59 and 71, and the LCDR1, LCDR2, and LCDR3 comprises the amino acid sequences of SEQ ID NOs:73, 87 and 98.

In one embodiment, the HCDR1, HCDR2, and HCDR3 comprises the amino acid sequences of SEQ ID NOs:44, 60 and 72, and the LCDR1, LCDR2, and LCDR3 comprises the amino acid sequences of SEQ ID NOs:86, 97 and 108.

In some embodiments, an isolated anti-IL1RAP antibody comprising three complementarity determining regions (CDRs) on a heavy chain (HCDR1, HCDR2, and HCDR3) and three CDRs on a light chain (LCDR1, LCDR2, and LCDR3), comprises any of the HCDR and LCDR sets for the antibodies presented in Tables 5A-5C.

In another embodiment, the anti-IL1RAP antibodies comprises heavy chain and light chain CDR sequences that are at least 80% (e.g., at least 85%, 90%, 95%, 96%, 97%, 98%, or 99%) identical to the amino acid sequences set forth in Tables 5A-5C.

In some embodiments, each of the anti-IL1RAP antibodies presented herein comprises a heavy chain variable region (VH) and a light chain variable region (VL), wherein the amino acid sequences for the heavy chain variable region and the light chain variable region can be one of the following pairs: SEQ ID NOs:1 and 18; SEQ ID NOs:2 and 19; SEQ ID NOs:3 and 20; SEQ ID NOs:4 and 21; SEQ ID NOs:5 and 22; SEQ ID NOs:6 and 23; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32. In another embodiment, the anti-IL1RAP antibodies comprise VH and VL sequences that are at least 80% (e.g., at least 85%, 90%, 95%, 96%, 97%, 98%, or 99%) identical to the VH and VL sequences set forth above. One skilled in the art would appreciate that percent sequence identity may be determined using any of a number of publicly available software application, for example but not limited to BlastP software of the National Center of Biotechnology Information (NCBI) using default parameters.

In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:1 and 18. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:2 and 19. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:3 and 20. In some embodiments, an anti-IL1RAP antibody comprises a heavy chain variable region (VH) and alight chain variable region (VL), wherein the amino acid sequences for the heavy chain variable region and the light chain variable region are set forth in SEQ ID NOs:4 and 21. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:5 and 22. In some embodiments, an anti-IL1RAP antibody comprises a heavy chain variable region (VH) and alight chain variable region (VL), wherein the amino acid sequences for the heavy chain variable region and the light chain variable region are set forth in SEQ ID NOs:6 and 23. In some embodiments, an anti-IL1RAP antibody comprises a heavy chain variable region (VH) and alight chain variable region (VL), wherein the amino acid sequences for the heavy chain variable region and the light chain variable region are set forth in SEQ ID NOs:6 and 22. In some embodiments, an anti-IL1RAP antibody comprises a heavy chain variable region (VH) and alight chain variable region (VL), wherein the amino acid sequences for the heavy chain variable region and the light chain variable region are set forth in or SEQ ID NOs:7 and 22. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:8 and 24. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:9 and 25. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:10 and 26. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:11 and 27. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:12 and 28. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:13 and 29. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:14 and 30. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:15 and 31. In some embodiments, an anti-IL1RAP antibody comprises a heavy chain variable region (VH) and a light chain variable region (VL), wherein the amino acid sequences for the heavy chain variable region and the light chain variable region are set forth in SEQ ID NOs:16 and 18. In some embodiments, an anti-IL1RAP antibody comprises a heavy chain variable region (VH) and alight chain variable region (VL), wherein the amino acid sequences for the heavy chain variable region and the light chain variable region are set forth in SEQ ID NOs:17 and 32.

In some embodiments, an anti-IL1RAP antibody comprises a heavy chain variable region (VH) and a light chain variable region (VL), wherein the amino acid sequences for the heavy chain variable region and the light chain variable region are set forth in any of the VH/VL sets presented for the antibodies of Tables 4A-4C, or comprise homologous sequences that are at least 80% (e.g., at least 85%, 90%, 95%, 96%, 97%, 98%, or 99%) identical to the VH and VL sequences set forth in Tables 4A-4C additionally indicates light chain type (κ vs. λ).

A skilled artisan would appreciate that the term “homology”, and grammatical forms thereof, encompasses the degree of similarity between two or more structures. The term “homologous sequences” refers to regions in macromolecules that have a similar order of monomers. Percent sequence identity is a number that describes how similar the query sequence is to the target sequence; with respect to amino acid sequences percent sequence identity indicates how many amino acid residues in each sequence are identical.

A skilled artisan would appreciate that an “IL1RAP binding antibody” encompasses in its broadest sense an antibody that specifically binds an antigenic determinant of an Interleukin-1 receptor accessory protein (IL1RAP) polypeptide. The skilled artisan would appreciate that specificity for binding to IL1RAP, reflects that the binding is selective for the IL1RAP antigen and can be discriminated from unwanted or nonspecific interactions. In certain embodiments, an IL1RAP binding antibody comprises an antibody fragment or fragments.

In some embodiments, an antigenic determinant comprises an IL1RAP epitope. The term “epitope” includes any determinant, in certain embodiments, a polypeptide determinant, capable of specific binding to an anti-IL1RAP binding domain. An epitope is a region of an antigen that is bound by an antibody or an antigen-binding fragment thereof. In some embodiments, an IL1RAP antigen-binding fragment of an antibody comprises a heavy chain variable region, a light chain variable region, or a combination thereof as described herein.

In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an IL1RAP binding antibody is said to specifically bind an IL1RAP epitope when it preferentially recognizes IL1RAP in a complex mixture of proteins and/or macromolecules.

In some embodiments, an IL1RAP binding antibody is said to specifically bind an IL1RAP epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

An antibody binding domain can be a fragment of an antibody or a genetically engineered product of one or more fragments of the antibody, which fragment is involved in specifically binding with the antigen. By “specifically binding” is meant that the binding is selective for the antigen of interest, for example for IL1RAP in embodiments described herein and can be discriminated from unwanted or nonspecific interactions. As used herein, the term “IL1RAP binding antibody” may in certain embodiments, encompass complete immunoglobulin structures, fragments thereof, or domains thereof.

In some embodiments, binding of an IL1RAP antibody disclosed herein, blocks of IL1R1/IL1β/IL1RAP complex formation. In some embodiments, binding of an IL1RAP antibody disclosed herein, inhibits IL1, IL33, and IL36 signaling in cancer cells. In some embodiments, an IL1RAP antibody disclosed herein, inhibits IL1 signaling in cancer cells. In some embodiments, an IL1RAP antibody inhibits the IL-1 signaling pathway, wherein IL-1 induces the synthesis of acute phase and proinflammatory proteins through activation of NFκB. In some embodiments, an IL1RAP antibody disclosed herein, inhibits IL33 signaling in cancer cells. In some embodiments, an IL1RAP antibody inhibits activates of the NFκB and MAP kinase signaling pathways that would enhance mast cell, TH2, regulatory T cell (Treg) and innate lymphoid cell type 2 functions. In some embodiments, an IL1RAP antibody disclosed herein, inhibits IL36 signaling in cancer cells. In some embodiments, an IL1RAP antibody inhibits IL36 activation of NFκB and MAP kinases that induce various inflammatory and skin diseases. In some embodiments, binding of an IL1RAP antibody disclosed herein, inhibits IL1, IL33, or IL36 signaling, or any combination thereof, in cancer cells. In some embodiments, binding of an IL1RAP antibody disclosed herein inhibits IL-1 induced secretion of IL-6. In some embodiments, binding of an IL1RAP antibody disclosed herein inhibits IL-36 induced secretion of IL-8. In some embodiments, binding of an IL1RAP antibody disclosed herein induces expression of macrophage differentiation markers. In some embodiments, binding of an IL1RAP antibody disclosed herein inhibits clonogenic capacity of cancer cells. In some embodiments, binding of an IL1RAP antibody disclosed herein inhibits proliferation of cancer cells. In some embodiments, binding of an IL1RAP antibody disclosed herein inhibits viability of cancer cells. In some embodiments, binding of an IL1RAP antibody disclosed herein inhibits clonogenic capacity, proliferation, and viability of cancer cells.

In some embodiments, binding of an IL1RAP antibody disclosed herein, reduces of IL1R/IL1β/IL1RAP complex formation. In some embodiments, binding of an IL1RAP antibody disclosed herein, reduces IL1, IL33, and IL36 signaling in cancer cells. In some embodiments, binding of an IL1RAP antibody disclosed herein, reduces IL1 signaling in cancer cells. In some embodiments, binding of an IL1RAP antibody disclosed herein, reduces IL33 signaling in cancer cells. In some embodiments, binding of an IL1RAP antibody disclosed herein, reduces IL36 signaling in cancer cells. In some embodiments, binding of an IL1RAP antibody disclosed herein, reduces IL1, 1L33, or IL36 signaling, or any combination thereof, in cancer cells. In some embodiments, binding of an IL1RAP antibody disclosed herein reduces IL-1 induced secretion of IL-6. In some embodiments, binding of an IL1RAP antibody disclosed herein reduces IL-36 induced secretion of IL-8. In some embodiments, binding of an IL1RAP antibody disclosed herein reduces clonogenic capacity of cancer cells. In some embodiments, binding of an IL1RAP antibody disclosed herein reduces proliferation of cancer cells. In some embodiments, binding of an IL1RAP antibody disclosed herein reduces viability of cancer cells. In some embodiments, binding of an IL1RAP antibody disclosed herein reduces clonogenic capacity, proliferation, and viability of cancer cells.

Examples of antibody binding domains include, without limitation, a complementarity determining region (CDR), a variable region (Fv), a VH domain, a light chain variable region (VL), a heavy chain, a light chain, a single chain variable region (scFv), and a Fab fragment. A skilled artisan would appreciate that an scFv is not actually a fragment of an antibody, but instead is a fusion polypeptide comprising the variable heavy chain (VH) and variable light chain (VL) regions of an immunoglobulin, connected by a short linker peptide of for example but not limited to ten to about 25 amino acids. The skilled artisan would also appreciate that the term “Fab” with regard to an antibody, generally encompasses that portion of the antibody consisting of a single light chain (both variable and constant regions) bound to the variable region and first constant region of a single heavy chain by a disulfide bond.

In some embodiments, an antibody encompasses whole antibody molecules, including monoclonal, polyclonal and multispecific (e.g., bispecific) antibodies. In some embodiments, an antibody encompasses an antibody fragment or fragments that retain binding specificity including, but not limited to, variable heavy chain (VH) fragments, variable light chain (VL) fragments, Fab fragments, F(ab′)2 fragments, scFv fragments, Fv fragments, minibodies, diabodies, triabodies, and tetrabodies (see, e.g., Hudson and Souriau, Nature Med. 9: 129-134 (2003) (hereby incorporated by reference in their entirety)). Also encompassed are humanized, primatized, and chimeric antibodies.

A skilled artisan would appreciate that an “isolated IL1RAP binding antibody”, in certain embodiments, encompasses an antibody that (1) is free of at least some other proteins with which it would typically be found in nature or with which it would typically be found during synthesis thereof, (2) is essentially free of other non-identical IL1RAP binding antibodies from the same source, (3) may be expressed recombinantly by a cell, (4) has been separated from at least about 50 percent of polynucleotides, lipids, carbohydrates, or other materials with which it is associated in during synthesis, or (5) does not occur in nature, or a combination thereof. Such an isolated antibody may be encoded by genomic DNA, cDNA, mRNA or other RNA, of may be of synthetic origin, or any combination thereof. In certain embodiments, the isolated antibody is substantially free from proteins or polypeptides or other contaminants that would interfere with its use (therapeutic, diagnostic, prophylactic, research or otherwise). As used throughout, the terms “IL1RAP antibody”, “IL1RAP binding antibody”, and the like, may be used interchangeably having all the same meanings and qualities.

In some embodiments, an IL1RAP antibody comprises a recombinant antibody. In some embodiments, an IL1RAP antibody comprises a humanized antibody. In some embodiments, an IL1RAP antibody comprises an engineered antibody. In certain embodiments, an engineered antibody comprises improved binding compared to available antibodies. In some embodiments, an engineered antibody comprises improved association and dissociation constants (Kₒₙ and K_{off}), compared to available other IL1RAP binding antibodies. In some embodiments, an engineered antibody comprises improved stability compared with available IL1RAP binding antibodies.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences that may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g. CH1, CH2, CH3, CH4, Ck, Cl).

In some embodiments, an anti-IL1RAP antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody. In some embodiments, an anti-IL1RAP antibody comprises an IgG. In some embodiments, an anti-IL1RAP antibody comprises an Fv. In some embodiments, an anti-IL1RAP antibody comprises an scFv. In some embodiments, an anti-IL1RAP antibody comprises an Fab. In some embodiments, an anti-IL1RAP antibody comprises an F(ab′)2. In some embodiments, an anti-IL1RAP antibody comprises a minibody. In some embodiments, an anti-IL1RAP antibody comprises a diabody. In some embodiments, an anti-IL1RAP antibody comprises a triabody. In some embodiments, an anti-IL1RAP antibody comprises a nanobody. In some embodiments, an anti-IL1RAP antibody comprises a single domain antibody. In some embodiments, an anti-IL1RAP antibody comprises a multi-specific antibody. In some embodiments, an anti-IL1RAP antibody comprises a bi-specific antibody. In some embodiments, an anti-IL1RAP antibody comprises a tri-specific antibody. In some embodiments, an anti-IL1RAP antibody comprises a single chain antibody. In some embodiments, an anti-IL1RAP antibody comprises heavy chain antibodies. In some embodiments, an anti-IL1RAP antibody comprises a chimeric antibody. In some embodiments, an anti-IL1RAP antibody comprises a humanized antibody.

In certain embodiments, the anti-IL1RAP antibody can be an IgG such as IgG1, IgG2, IgG3, or IgG4. In some embodiments, an anti-IL1RAP antibody comprise an IgG1. In some embodiments, an anti-IL1RAP antibody comprise an IgG2. In some embodiments, an anti-IL1RAP antibody comprise an IgG3. In some embodiments, an anti-IL1RAP antibody comprise an IgG4.

In one embodiment, in view of the sequences for the heavy chain variable regions and light chain variable regions disclosed herein, one of ordinary skill in the art would readily employ standard techniques known in the art to construct an anti-IL1RAP scFv.

In some embodiments, use of an anti-IL1RAP antibody or a composition comprising an anti-IL1RAP antibody induces antibody-dependent cell-mediated cytotoxicity (ADCC).

A skilled artisan would appreciate that ADCC may also be referred to as antibody-dependent cellular cytotoxicity. In some embodiments, use of an anti-IL1RAP antibody or a composition comprising an anti-IL1RAP antibody induces ADCC, wherein a target cell is lysed, or other types of cytotoxicity occur including Complement Dependent Cytotoxicity (CDC) or Complement Dependent Phagocytosis (CDP).

In some embodiments, use of an anti-IL1RAP antibody comprises use of IL1RAP antibody-drug conjugate (ADC). In some embodiments, use of an IL1RAP antibody composition comprises use of an IL1RAP ADC composition. In some embodiments, use of an anti-IL1RAP ADC results in cytotoxicity of the targeted cells. In some embodiments, an anti-IL1RAP ADC is used for therapeutic and or prophylactic purposes to treat cancer.

As used herein, “antibody-dependent cellular cytotoxicity” (ADCC), also referred to as “antibody-dependent cell-mediated cytotoxicity,” is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. ADCC requires an effector cell which classically is known to be natural killer (NK) cells that typically interact with immunoglobulin G (IgG) antibodies. However, macrophages, neutrophils and eosinophils can also mediate ADCC.

As used herein, a “natural killer cell” (NK cell or NKC), also known as large granular lymphocyte (LGL), is a type of cytotoxic lymphocyte critical to the innate immune system that belongs to the family of innate lymphoid cells (ILC). NK cells also play a role in the adaptive immune response.

In some embodiments, an anti-IL1RAP antibody comprises a mutated immunoglobulin. Examples of mutated immunoglobulins include immunoglobulins where the Fc portion has been engineered. The cellular immune response occurs mostly due to the interactions between the antibody and Fc gamma receptors (FcγRs). Non-limiting examples of immunoglobulins wherein the Fc portion of an immunoglobulin has been engineered is provided at least in Wang et al., (2018) Protein Cell, 9(1):63-73 (See Table 1 of Wang et al.) and Liu R, et al., (2020) Fc-Engineering for Modulated Effector Functions-Improving Antibodies for Cancer Treatment. Antibodies (Basel).9(4):64, incorporated herein in full. Examples of mutated immunoglobulins, wherein binding of an IgG with cellular cytotoxicity (ADCC) components is altered may be found for example in Xu D, Alegre M L, Varga S S, Rothermel A L, Collins A M, Pulito V L, et al. In vitro characterization of five humanized OKT3 effector function variant antibodies. Cell Immunol. (2000) 200:16-26, incorporated herein in full. In some embodiments, an anti-IL1RAP immunoglobulin comprises an engineered Fc portion such that the interaction between the antibody and an Fc gamma receptor is increased, decreased, or eliminated.

Several mutations in the Fc chain have been shown to increase binding to Fcγ receptors and complement proteins. In some embodiments, an anti-IL1RAP antibody comprising mutations in the Fc chain exhibits enhanced ADCC activity. In some embodiments, an anti-IL1RAP antibody comprising mutations in the Fc chain exhibits enhanced CDC activity. In some embodiments, mutations comprise point mutations.

Fucose removal has been shown to significantly enhance ADCC for certain antibodies, via improved binding to Fcγ receptors. In some embodiments, an anti-IL1RAP antibody is afucosylated. In some embodiments, an anti-IL1RAP antibody comprises a reduced % of fucose sugars on the Fc region compared with an antibody that has not had fucose sugars removed from the Fc region or had fucose incorporation to the Fc region limited

In some embodiments, an anti-IL1RAP antibody comprises mutations in the Fc chain and is afucosylated. In some embodiments, an anti-IL1RAP antibody comprises mutations in the Fc chain and comprises reduced % of fucose sugars on the Fc region compared with an antibody that has not had fucose sugars removed from the Fc region or had fucose incorporation to the Fc region limited

In one embodiment, in view of the sequences for the heavy chain variable regions and light chain variable regions disclosed herein, one of ordinary skill in the art would readily employ standard techniques known in the art to construct an anti-IL1RAP wherein the Fc region comprises mutations that enhance ADCC activity and or wherein the Fc region is afucosylated. In some embodiments, use of a modified anti-IL1RAP antibody comprising mutations and or reduced fucose in the Fc region, or a composition thereof enhances induction of ADCC activity.

In some embodiments, an Fc region modification comprises substitution mutations comprising S298A/E333A/K334A or S239D/I332E or S239D/A330L/I332E or G236A or G236A/S239D/I332E or G236A/A330L/I332E or G236A/S239D/A330L/I332E or F243L/R292P/Y300L/V305I/P396L or L235V/F243L/R292P/Y300L/P396L. In some embodiments, an Fc region modification comprises afucosylation. In some embodiments, an Fc region modification comprises a combination of any of substitution mutations comprising S298A/E333A/K334A or S239D/I332E or S239D/A330L/I332E or G236A or G236A/S239D/I332E or G236A/A330L/I332E or G236A/S239D/A330L/I332E or F243L/R292P/Y300L/V305I/P396L or L235V/F243L/R292P/Y300L/P396L with afucosylation.

In one embodiment, the present disclosure provides antibodies that bind with high affinity to IL1RAP. In one embodiment, binding affinity is calculated by a modification of the Scatchard method as described by Frankel et al. (Mol. Immunol., 16:101-106, 1979). In another embodiment, binding affinity is measured by an antigen/antibody dissociation rate. In another embodiment, binding affinity is measured by a competition radioimmunoassay. In another embodiment, binding affinity is measured by ELISA. In another embodiment, antibody affinity is measured by flow cytometry.

### Polynucleotides, Vectors, and Host Cells

In one embodiment, the present disclosure also provides isolated polynucleotide sequence encoding the heavy chain and light chain CDRs as described herein, for example as set forth in Tables 5A-5C. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

In one embodiment, the present disclosure also provides isolated polynucleotide sequence encoding the heavy chain and light chain variable regions described herein, for example as set forth in Tables 4A-4C. In another embodiment, the present disclosure also provides a vector comprising such polynucleotide sequences. In view of the amino acid sequences disclosed herein, one of ordinary skill in the art would readily construct a vector or plasmid to encode for the amino acid sequences. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

One skilled in the art would appreciate that the polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

In certain embodiments, the isolated polynucleotide is inserted into a vector. The term “vector” as used herein encompasses a vehicle into which a polynucleotide encoding a protein may be covalently inserted so as to bring about the expression of that protein and/or the cloning of the polynucleotide. The isolated polynucleotide may be inserted into a vector using any suitable methods known in the art, for example, without limitation, the vector may be digested using appropriate restriction enzymes and then may be ligated with the isolated polynucleotide having matching restriction ends.

Examples of suitable vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). In some embodiments, said vector comprises an expression vector.

In some embodiments, an expression vector comprises a nucleic acid construct described herein. Suitable vectors can be chosen or constructed, containing appropriate regulatory sequences, including promoter sequences, terminator sequences, polyadenylation sequences, enhancer sequences, marker genes and other sequences as appropriate.

Regulatory sequences may be operably linked to the nucleic acid sequence(s) comprised within a nucleic acid construct. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detailing Current Protocols in Molecular Biology, Second Edition, Ausubel et al. eds., John Wiley & Sons, 1988, Short Protocols in Molecular Biology: A Compendium of Methods from Current Protocols in Molecular Biology, Ausubel et al. eds., John Wiley & Sons, 4.sup.th edition 1999. The disclosures of Sambrook et al. and Ausubel et al. (both) are incorporated herein by reference.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art.

For expression of the IL1RAP antibody or components thereof, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. In some embodiments, these elements may be considered “control” elements.

A skilled artisan would appreciate that the term “control sequence” may encompass polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

In some embodiments, for example but not limited to, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

A vector may also include materials to aid in its entry into the cell, including but not limited to a viral particle, a liposome, or a protein coating.

In some embodiments, an expression vector comprises an isolated polynucleotide sequence encoding an IL1RAP antibody or a component thereof, for example but not limited to a VH domain, a VL domain, a combined VH-VL domain as may be present in Fab elements, F(ab′)2 elements, an IgG, an Fv, or an scFv. In some embodiments, an expression vector comprises a polynucleotide sequence encoding IL1RAP HCDR or LCDR domains, or a combination thereof as set forth in Tables 5A-5C. In some embodiments, an expression vector comprises a polynucleotide sequence encoding an IL1RAP VH domain or VL domain, or a combination thereof, as set forth in Tables 4A-4C. In some embodiments, an isolated polynucleotide sequence encodes a component of an anti-IL1RAP antibody component of a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof, as described above. IL1RAP binding domains and the components thereof have been described in detail above.

In some embodiments, an expression vector comprises an isolated polynucleotide sequence encoding a VH domain. In some embodiments, an expression vector comprises an isolated nucleic acid sequence encoding a VL domain. In some embodiments, an expression vector comprises an isolated nucleic acid sequence encoding a VH and a VL domain. In some embodiments, an expression vector comprises an isolated nucleic acid sequence encoding set of CDR's of a VH region. In some embodiments, an expression vector comprises an isolated nucleic acid sequence encoding set of CDR's of a VL region. In some embodiments, an expression vector comprises an isolated nucleic acid sequence encoding set of CDR's of a VH region and a VL region.

In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide(s) contained in the vector.

In some embodiments, a recombinant host cell comprises one or more constructs as described above. A polynucleotide encoding any CDR or set of CDR's or VH domain or VL domain or antibody antigen-binding site or antibody molecule, for example but not limited to an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In some embodiments, a host cell comprises one or more constructs as described above encoding an IgG subclass selected from an IgG1, IgG2, IgG3, and IgG4.

In some embodiments, disclosed herein is a method of production of the encoded product, which method comprises expression from the polynucleotide constructs. In some embodiments, a polynucleotide construct comprises a polynucleotide sequence encoding the HCDR or LCDR sequences or a combination thereof as set forth in Tables 5A-5C. In some embodiments, a polynucleotide construct comprises a polynucleotide sequence encoding the VH or VL sequences or a combination thereof as set forth in Tables 4A-4C. Expression may in some embodiments, be achieved by culturing under appropriate conditions recombinant host cells containing the nucleic acid construct. Following production by expression, an antibody or an IL1RAP antigen-binding fragment thereof, may be isolated and/or purified using any suitable technique, then used as appropriate, for example in methods of treatment as described herein.

In some embodiments, systems for cloning and expression of a polypeptide in a variety of different host cells are well known. Suitable host cells can include, without limitation, prokaryotic cells, fungal cells, yeast cells, or higher eukaryotic cells such as insect cells or mammalian cells.

Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable fungal cells for this purpose include, without limitation, filamentous fungi and yeast. Illustrative examples of fungal cells include, Saccharomyces cerevisiae, common baker's yeast, Schizosaccharomyces pombe, Kluyveromyces hosts such as, eg., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Higher eukaryotic cells, in particular, those derived from multicellular organisms can be used for expression of glycosylated VH and VL domains, as provided herein. Suitable higher eukaryotic cells include, without limitation, invertebrate cells and insect cells, and vertebrate cells. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the K-1 variant of Autographa californica NPV and the Bm-5 strain of Bombyx mori NPV, and such viruses may be used as the virus herein as described herein, particularly for transfection of Spodoptera frugiperda cells. Plant cell cultures of cotton, corn, potato, soybean, petunia, tomato, and tobacco can also be utilized as hosts. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney cells, human embryonic retina cells and many others. Non-limiting examples of vertebrate cells include mammalian host cell lines such as monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); ExpiCHO-S™ cells (ThermoFisher Scientific cat. #A29133); mouse Sertoli cells (TM4, Mather, Biol. Reprod. 23:243-251 (1980)); monkey kidney cells (CV1 ATCC CCL 70); African green monkey kidney cells (VERO-76, ATCC CRK-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

A non-limiting example of an expression system well known in the art is the Lonza (USA) GS Gene Expression System®. In some embodiments, a vector encoding a polypeptide described herein comprises a GS® vector of Lonza (USA), for example but not limited to pXC-IgG1zaDK (based on pXC-18.4) and pXC-Kappa (based on pXC-17.4). These GS® vectors and other similar vectors known in the art, include a range of vector choices comprising Universal base vectors, IgG constant region vectors, IgG site-specific conjugation vectors, pXC Multigene vectors, and GS piggyBac™ vectors (+transposase). In some embodiments, a host cell from which an encoded polypeptide described herein may be expressed comprises a GS Xceed® CHOK1SV GS-KO® cell line or other similar cell known known in the art or created for the purpose of optimizing protein expression. In some embodiments, the combination of vector and host cell optimizes expression of IL1RAP antibody polypeptides or IL1RAP binding fragments thereof.

In some embodiments, provided herein is a host cell containing nucleic acid as disclosed herein. Such a host cell may be in vitro and may be in culture. Such a host cell may be in vivo. In vivo presence of the host cell may allow intracellular expression of IL1RAP binding antibodies described herein, as “intrabodies” or intracellular antibodies. Intrabodies may be used for gene therapy.

In certain embodiments, the host cells comprise a first vector encoding a first polypeptide, e.g., a VH domain, and a second vector encoding a second polypeptide, e.g., a VL domain. In certain embodiments, the host cells comprise a vector encoding a first polypeptide, e.g., a VH domain, and a second polypeptide, e.g., a VL domain.

In certain embodiments, the host cells comprise a first vector encoding a VH domain and a second vector encoding a VL domain. In certain embodiments, the host cells comprise a single vector encoding a VH domain and a VL domain.

In some embodiments, an isolated cell comprises an isolated nucleic acid sequence, as disclosed herein. In some embodiments, an isolated cell comprises two isolated nucleic acid sequences as disclosed herein, wherein one nucleic acid encodes a VH domain and the other nucleic acid encodes a VL domain. In some embodiments, an isolated cell comprises a single isolated nucleic acid sequences as disclosed herein, that encodes a VH domain and a VL domain.

In certain embodiments, a first vector and a second vector may or may not be introduced simultaneously. In certain embodiments, the first vector and the second vector may be introduced together into the host cell. In certain embodiments, the first vector may be introduced first into the host cell, and then the second vector may be introduced. In certain embodiments, the first vector may be introduced into the host cell, which is then established into a stable cell line expressing the first polypeptide, and then the second vector may be introduced into the stable cell line.

The introduction may be followed by causing or allowing expression from the nucleic acid, e.g. by culturing host cells under conditions for expression of the gene. In certain embodiments, the present disclosure provides methods of expressing the polypeptide provided herein, comprising culturing the host cell containing the vector under conditions in which the inserted polynucleotide in the vector is expressed.

In some embodiments, the nucleic acid is integrated into the genome (e.g. chromosome) of the host cell. Integration may be promoted by inclusion of sequences which promote recombination with the genome, in accordance with standard techniques. In some embodiments, the nucleic acid construct is not integrated into the genome and the vector is episomal.

In some embodiments, disclosed herein is a method which comprises using a construct as stated above in an expression system in order to express an IL1RAP binding antibody or fragment thereof, as described herein above.

Suitable conditions for expression of the polynucleotide may include, without limitation, suitable medium, suitable density of host cells in the culture medium, presence of necessary nutrients, presence of supplemental factors, suitable temperatures and humidity, and absence of microorganism contaminants. A person with ordinary skill in the art can select the suitable conditions as appropriate for the purpose of the expression.

In some embodiments, disclosed herein are methods of producing an antiIL1RAP antibody comprises expressing the vector comprising any of the anti-IL1RAP antibodies disclosed herein or a fragment thereof, in a host cell under conditions conducive to expressing said vector in said host cell, thereby producing an anti-IL1RAP antibody.

In some embodiments, IL1RAP binding antibodies described herein may be prepared and isolated and/or purified, in substantially pure or homogeneous form. In some embodiments, disclosed herein is a method of producing an anti-IL1RAP antibody comprising a heavy chain variable region (VH) and a light chain variable region (VH), comprises the step of culturing a host cell under conditions conducive to expressing a vector in said host cell, thereby expressing a polynucleotide sequence comprised in the vector and thereby producing an anti-IL1RAP antibody or an IL1RAP antigen binding domain thereof. In some embodiments, disclosed herein is a method of producing an anti-IL1RAP antibody comprising a heavy chain variable region (VH) and a light chain variable region (VH), comprises the step of culturing a host cell comprising a vector comprising an isolated polynucleotide sequence encoding the heavy chain variable region (VH) of an anti-IL1RAP antibody and the light chain variable region (VL) of the anti-IL1RAP antibody, wherein the amino acid sequence of the VH-VL pair are selected from the paired sequences set forth in Tables 4A-4C; under conditions conducive to expressing a vector in said host cell, thereby expressing a polynucleotide sequence comprised in the vector and thereby producing an anti-IL1RAP antibody comprising a VH and VL or an IL1RAP antigen binding domain thereof.

In some embodiments, disclosed herein is a method of producing an anti-IL1RAP antibody comprising complementarity determining region (CDR) sequences as set forth in Tables 5A-5C, the method comprising the step of culturing a host cell comprising a vector comprising an isolated polynucleotide sequence encoding a heavy chain variable region (VH) of an anti-IL1RAP antibody comprising the complementarity determining regions (HCDR) of said VH as set forth in Table 5B and a light chain variable region (VL) of an anti-IL1RAP antibody comprising the complementarity determining regions (LCDR) of said VL as set forth in Table 5C, said heavy chain variable region having heavy chain complementarity determining region (HCDR) 1, HCDR2 and HCDR3, and said light chain variable region having light chain complementarity determining region (LCDR) 1, LCDR2 and LCDR3, wherein said HCDR1, HCDR2, HCDR3, LCDR1, LCDR2 and LCDR3 for said antibody comprise those set forth in Tables 5A-5C, respectively; under conditions conducive to expressing said vector in said host cell, and expressing said polynucleotide sequences comprised in said vector, thereby producing an anti-IL1RAP antibody having complementarity determining region (CDR) sequences as set forth in Tables 5A-5C.

In some embodiments of a method for producing an IL1RAP antibody, the antibody is produced in vivo. In some embodiments of a method for producing an IL1RAP antibody, the antibody is produced in vitro. In some embodiments of a method for producing an IL1RAP antibody, when the antibody is produced in vitro it may in a further step be isolated.

### Compositions for Use

In some embodiments, the present disclosure also provides a composition comprising the anti-IL1RAP antibody disclosed herein and a pharmaceutically acceptable carrier. Pharmaceutically acceptable carriers of use are well-known in the art. For example, Remington's Pharmaceutical Sciences, by E. W. Martin, Mack Publishing Co., Easton, PA, 15th Edition, 1975, describes compositions and formulations suitable for pharmaceutical delivery of the antibodies disclosed herein. In some embodiments, the composition comprises anti-IL1RAP antibodies that comprise a set of three complementarity determining regions (CDRs) on a heavy chain (HCDR1, HCDR2, and HCDR3) and a set of three CDRs on a light chain (LCDR1, LCDR2, and LCDR3).

In some embodiments, the HCDR1, HCDR2, and HCDR3 comprises the amino acid sequences of SEQ ID NOs:33, 45 and 61, and the LCDR1, LCDR2, and LCDR3 comprises the amino acid sequences of SEQ ID NOs:73, 87 and 98.

In some embodiments, the HCDR1, HCDR2, and HCDR3 comprises the amino acid sequences of SEQ ID NOs:33, 46 and 62, and the LCDR1, LCDR2, and LCDR3 comprises the amino acid sequences of SEQ ID NOs:74, 87 and 99.

In some embodiments, the HCDR1, HCDR2, and HCDR3 comprises the amino acid sequences of SEQ ID NOs:33, 47 and 62, and the LCDR1, LCDR2, and LCDR3 comprises the amino acid sequences of SEQ ID NOs:75, 88 and 100.

In some embodiments, the HCDR1, HCDR2, and HCDR3 comprises the amino acid sequences of SEQ ID NOs:34, 48 and 63, and the LCDR1, LCDR2, and LCDR3 comprises the amino acid sequences of SEQ ID NOs:76, 89 and 101.

In some embodiments, the HCDR1, HCDR2, and HCDR3 comprises the amino acid sequences of SEQ ID NOs:35, 49 and 64, and the LCDR1, LCDR2, and LCDR3 comprises the amino acid sequences of SEQ ID NOs:77, 90 and 102.

In some embodiments, the HCDR1, HCDR2, and HCDR3 comprises the amino acid sequences of SEQ ID NOs:35, 50 and 65, and the LCDR1, LCDR2, and LCDR3 comprises the amino acid sequences of SEQ ID NOs:77, 90 and 102.

In some embodiments, the HCDR1, HCDR2, and HCDR3 comprises the amino acid sequences of SEQ ID NOs:35, 51 and 64, and the LCDR1, LCDR2, and LCDR3 comprises the amino acid sequences of SEQ ID NOs:77, 90 and 102.

In some embodiments, the HCDR1, HCDR2, and HCDR3 comprises the amino acid sequences of SEQ ID NOs:36, 50 and 64, and the LCDR1, LCDR2, and LCDR3 comprises the amino acid sequences of SEQ ID NOs:78, 90 and 102.

In some embodiments, the HCDR1, HCDR2, and HCDR3 comprises the amino acid sequences of SEQ ID NOs:37, 52 and 66, and the LCDR1, LCDR2, and LCDR3 comprises the amino acid sequences of SEQ ID NOs:79, 91 and 103.

In some embodiments, the HCDR1, HCDR2, and HCDR3 comprises the amino acid sequences of SEQ ID NOs:38, 53 and 67, and the LCDR1, LCDR2, and LCDR3 comprises the amino acid sequences of SEQ ID NOs:80, 92 and 104.

In some embodiments, the HCDR1, HCDR2, and HCDR3 comprises the amino acid sequences of SEQ ID NOs:39, 54 and 67, and the LCDR1, LCDR2, and LCDR3 comprises the amino acid sequences of SEQ ID NOs:81, 92 and 104.

In some embodiments, the HCDR1, HCDR2, and HCDR3 comprises the amino acid sequences of SEQ ID NOs:40, 55 and 68, and the LCDR1, LCDR2, and LCDR3 comprises the amino acid sequences of SEQ ID NOs:82, 93 and 105.

In some embodiments, the HCDR1, HCDR2, and HCDR3 comprises the amino acid sequences of SEQ ID NOs:41, 56 and 69, and the LCDR1, LCDR2, and LCDR3 comprises the amino acid sequences of SEQ ID NOs:83, 94 and 106.

In some embodiments, the HCDR1, HCDR2, and HCDR3 comprises the amino acid sequences of SEQ ID NOs:42, 57 and 70, and the LCDR1, LCDR2, and LCDR3 comprises the amino acid sequences of SEQ ID NOs:84, 95 and 107.

In some embodiments, the HCDR1, HCDR2, and HCDR3 comprises the amino acid sequences of SEQ ID NOs:43, 58 and 70, and the LCDR1, LCDR2, and LCDR3 comprises the amino acid sequences of SEQ ID NOs:85, 96 and 107.

In some embodiments, the HCDR1, HCDR2, and HCDR3 comprises the amino acid sequences of SEQ ID NOs:44, 59 and 71, and the LCDR1, LCDR2, and LCDR3 comprises the amino acid sequences of SEQ ID NOs:73, 87 and 98.

In some embodiments, the HCDR1, HCDR2, and HCDR3 comprises the amino acid sequences of SEQ ID NOs:44, 60 and 72, and the LCDR1, LCDR2, and LCDR3 comprises the amino acid sequences of SEQ ID NOs:86, 97 and 108.

In other embodiments, the composition comprises anti-IL1RAP antibodies having heavy chain and light chain CDR sequences that are at least 80% (e.g., at least 85%, 90%, 95%, 96%, 97%, 98%, or 99%) identical to the amino acid sequences set forth above.

In some embodiments, the composition comprises anti-IL1RAP antibodies having one of the following pairs of heavy chain variable region and light chain variable region: SEQ ID NOs:1 and 18; SEQ ID NOs:2 and 19; SEQ ID NOs:3 and 20; SEQ ID NOs:4 and 21; SEQ ID NOs:5 and 22; SEQ ID NOs:6 and 23; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32. In another embodiment, the composition comprises anti-IL1RAP antibodies having VH and VL sequences that are at least 80% (e.g., at least 85%, 90%, 95%, 96%, 97%, 98%, or 99%) identical to the amino acid sequences set forth above.

In some embodiments of compositions, the antibodies disclosed herein can be in the form of a conjugate. As used herein, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate can also be referred to as an “immunoconjugate.” When the conjugate comprises an antibody linked to a drug (e.g., a cytotoxic agent), the conjugate can be referred to as an “antibody-drug conjugate”. Other antibody conjugates include, for example, multi-specific (such as bispecific or trispecific) antibodies and chimeric antigen receptors (CARs).

A composition comprising the anti-IL1RAP antibody or an antigen-binding fragment thereof can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The pharmaceutical compositions comprising the antibodies or antigen-binding fragments thereof disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Alternatively, administration can be intratumoral, e.g., local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

### Methods of Use

In some embodiments, the anti-IL1RAP antibodies disclosed herein can be used to treat a disease or condition. In some embodiments, the disease comprises a cancer or tumor, an autoimmune disease, or GvHD. In some embodiments, uses of an anti-IL1RAP antibody described herein include use as an immunotherapeutic agent. In some embodiments, the anti-IL1RAP antibodies disclosed herein can be used to treat diseases such as cancer. In some embodiments, the anti-IL1RAP antibodies disclosed herein can be used as a component of a vaccine. In some embodiments, the anti-IL1RAP antibodies disclosed herein can be used as part of an antibody-drug conjugate (ADC). In some embodiments, a IL1RAP antibody described herein may be used in methods of treating a disease or condition, wherein said disease or condition comprises an inflammatory disease. In some embodiments, a IL1RAP antibody described herein may be used in methods of treating a disease or condition, wherein said disease or condition comprises a skin disease. In some embodiments, a skin disease comprises psoriasis. In some embodiments, a IL1RAP antibody described herein may be used in methods of treating a disease or condition, wherein said disease or condition comprises rheumatic disease. In some embodiments, a IL1RAP antibody described herein may be used in methods of treating a disease or condition, wherein said disease or condition comprises an acute myocardial infarction. In some embodiments, a IL1RAP antibody described herein may be used in methods of treating a disease or condition, wherein said disease or condition comprises asthma. In some embodiments, a IL1RAP antibody described herein may be used in methods of treating a disease or condition, wherein said disease or condition comprises eosinophilic pneumonia. In some embodiments, a IL1RAP antibody described herein may be used in methods of treating a disease or condition, wherein said disease or condition comprises psoriatic arthritis, systemic lupus, inflammatory bowel disease, ulcerative colitis, Crohn's disease, or Sjögren's syndrome.

In some embodiments, an anti-IL1RAP antibody disclosed herein can be used in methods of treating cancer, for example but not limited to treating non-small-cell lung carcinoma (NSCLC), breast cancer, mesothelioma, pancreatic cancer, renal cancer, prostate cancer, ovarian cancer, or colon cancer.

In some embodiments, the anti-IL1RAP antibodies used in a method to treat a disease or condition comprise three complementarity determining regions (CDRs) on a heavy chain (HCDR1, HCDR2, and HCDR3) and three CDRs on a light chain (LCDR1, LCDR2, and LCDR3), comprises any of the HCDR and LCDR sets for the antibodies presented in Tables 5A-5C. In some embodiments, the anti-IL1RAP antibodies used in a method to treat a disease or condition comprise a heavy chain variable region (VH) and a light chain variable region (VL), wherein the amino acid sequences for the heavy chain variable region and the light chain variable region can be one of the following pairs: SEQ ID NOs:1 and 18; SEQ ID NOs:2 and 19; SEQ ID NOs:3 and 20; SEQ ID NOs:4 and 21; SEQ ID NOs:5 and 22; SEQ ID NOs:6 and 23; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32. In another embodiment in method of use of treating a disease or condition, the anti-IL1RAP antibodies or compositions thereof comprise VH and VL sequences that are at least 80% (e.g., at least 85%, 90%, 95%, 96%, 97%, 98%, or 99%) identical to the VH and VL sequences set forth above.

In some embodiments, the anti-IL1RAP antibodies disclosed herein can be used to treat a disease associated with IL1RAP. In some embodiments, the anti-IL1RAP antibodies disclosed herein can be used to treat a disease associated with over-expression of IL1RAP.

In some embodiments, the anti-IL1RAP antibodies disclosed herein comprise cytotoxic activities. In some embodiments, the anti-IL1RAP antibodies disclosed herein are cytotoxic to cancer or tumor cells.

In some embodiments, the anti-IL1RAP antibodies disclosed herein may be used in a method to a cancer or tumor. In some embodiments, methods of use of an anti-IL1RAP antibody or a composition thereof, comprise for example, inhibiting tumor formation or growth, or a combination thereof. In some embodiments, methods of use of an anti-IL1RAP antibody or a composition thereof, comprise inhibiting or reducing tumor cell proliferation. In some embodiments, methods of use of an anti-IL1RAP antibody or a composition thereof, comprise inhibiting or reducing tumor cell viability. In some embodiments, methods of use of an anti-IL1RAP antibody or a composition thereof, comprise inhibiting or reducing tumor cell clonogenicity.

Cell viability may be assessed by known techniques, such as trypan blue exclusion assays. Viability or conversely, toxicity, may also be measured based on cell viability, for example the viability of normal and cancerous cell cultures exposed to the anti-IL1RAP antibody may be compared. Toxicity may also be measured based on cell lysis, for example the lysis of normal and cancerous cell cultures exposed to the anti-IL1RAP antibody may be compared. Cell lysis may be assessed by known techniques, such as Chromium (Cr) release assays or dead cell indicator dyes (propidium Iodide, TO-PRO-3 Iodide).

In some embodiments, disclosed herein is a method of inhibiting tumor formation or growth or a combination thereof in a subject in need, the method comprising the step of administering to said subject an anti-IL1RAP antibody as disclosed herein comprising an antibody antigen-binding domain comprising a heavy chain variable region (VH) and a light chain variable region (VL), wherein the amino acid sequences of a VH-VL pair are selected from the paired sequences: SEQ ID NOs:1 and 18; SEQ ID NOs:2 and 19; SEQ ID NOs:3 and 20; SEQ ID NOs:4 and 21; SEQ ID NOs:5 and 22; SEQ ID NOs:6 and 23; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32, thereby inhibiting tumor formation or growth or a combination thereof in said subject. In another embodiment, in methods of inhibiting tumor formation or growth or a combination thereof, the anti-IL1RAP antibodies or compositions thereof comprise VH and VL sequences that are at least 80% (e.g., at least 85%, 90%, 95%, 96%, 97%, 98%, or 99%) identical to the VH and VL sequences set forth above.

In some embodiments, disclosed herein is a method of inhibiting tumor formation or growth or a combination thereof in a subject in need, comprising the step of administering to said subject an anti-IL1RAP antibody having complementarity determining region (CDR) sequences as set forth in Tables 5A-5C or a composition thereof, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprises the CDR sets of amino acid sequences as set forth in Tables 5A-5C, thereby inhibiting tumor formation or growth or a combination thereof in said subject.

In some embodiments, a method of inhibiting tumor formation or growth or both inhibits tumor formation. In some embodiments, a method of inhibiting tumor formation or growth or both reduces the rate of tumor formation. In some embodiments, a method of inhibiting tumor formation or growth or both inhibits tumor growth. In some embodiments a method of inhibiting tumor formation or growth or both reduces the rate of tumor growth. In some embodiments, a method of inhibiting tumor formation or growth or both halts tumor growth. In some embodiments, a method of inhibiting tumor formation or growth or both inhibits tumor formation de novo and reduces the growth of a tumor. In some embodiments, a method of inhibiting tumor formation or growth or both reduces the rate of tumor formation de novo and reduces the growth of a tumor. In some embodiments, a method of inhibiting tumor formation or growth or both inhibits tumor formation de novo, inhibits the growth of a tumor, and inhibits metastasis. In some embodiments, a method of inhibiting tumor formation or growth or both reduces the rate of tumor formation de novo, reduces the growth of a tumor, and reduces the rate of tumor metastasis. In some embodiments, a method of inhibiting tumor formation or growth or both inhibits tumor metastasis. In some embodiments a method of inhibiting tumor formation or growth or both reduces the rate of tumor metastasis.

In some embodiments, the cancer or tumor comprises a solid cancer or tumor. In some embodiments, a solid tumor comprises an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors. In some embodiments, a solid tumor comprises a sarcoma or a carcinoma.

In some embodiments, solid tumors are neoplasms (new growth of cells) or lesions (damage of anatomic structures or disturbance of physiological functions) formed by an abnormal growth of body tissue cells other than blood, bone marrow or lymphatic cells. In some embodiments, a solid tumor consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In some embodiments, examples of solid tumors comprise sarcomas, carcinomas, and lymphomas. In some embodiments, a solid tumor comprises a sarcoma or a carcinoma. In some embodiments, the solid tumor is an intra-peritoneal tumor.

In some embodiments, a cancer or a tumor comprises a high-risk myelodysplastic syndromes (MDS). In some embodiments, methods of treating a cancer or a tumor comprises treating a cancer or tumor having increased IL-1 expression, for example but not limited to pancreatic, head and neck, lung, breast, colon, and melanomas.

In some embodiments, a solid tumor comprises, but is not limited to, lung cancer, breast cancer, ovarian cancer, stomach cancer, esophageal cancer, cervical cancer, head and neck cancer, bladder cancer, liver cancer, and skin cancer. In some embodiments, a solid tumor comprises a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

In some embodiments, the solid tumor comprises an Adrenocortical Tumor (Adenoma and Carcinoma), a Carcinoma, a Colorectal Carcinoma, a Desmoid Tumor, a Desmoplastic Small Round Cell Tumor, an Endocrine Tumor, an Ewing Sarcoma, a Germ Cell Tumor, a Hepatoblastoma a Hepatocellular Carcinoma, a Melanoma, a Neuroblastoma, an Osteosarcoma, a Retinoblastoma, a Rhabdomyosarcoma, a Soft Tissue Sarcoma Other Than Rhabdomyosarcoma, and a Wilms Tumor. In some embodiments, the solid tumor is a breast tumor. In another embodiment, the solid tumor is a prostate cancer. In another embodiment, the solid tumor is a colon cancer. In some embodiments, the tumor is a brain tumor. In another embodiment, the tumor is a pancreatic tumor. In another embodiment, the tumor is a colorectal tumor.

In some embodiments, anti-IL1RAP antibodies or compositions thereof as disclosed herein, have therapeutic and/or prophylactic efficacy against a cancer or a tumor, for example sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some embodiments, a method of treating a disease or condition comprises treating a solid cancer or solid tumor comprising a sarcoma, an osteosarcoma, a squamous cell carcinoma of the head and neck, a non-small-cell lung carcinoma, a bladder cancer, a pancreatic cancer, or a pancreatic ductal adenocarcinoma.

In some embodiments, the cancer or tumor comprises a non-solid (diffuse) cancer or tumor. Examples of diffuse cancers include leukemias. Leukemias comprise a cancer that starts in blood-forming tissue, such as the bone marrow, and causes large numbers of abnormal blood cells to be produced and enter the bloodstream.

In some embodiments, a diffuse cancer comprises a B-cell malignancy. In some embodiments, the diffuse cancer comprises leukemia. In some embodiments, the cancer is lymphoma. In some embodiments, the lymphoma is large B-cell lymphoma.

In some embodiments, the diffuse cancer or tumor comprises a hematological tumor. In some embodiments, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and masT-cells, whereas the lymphoid cell line produces B, T, NK and plasma cells. Lymphomas (e.g., Hodgkin's Lymphoma), lymphocytic leukemias, and myeloma are derived from the lymphoid line, while acute and chronic myelogenous leukemia (AML, CML), myelodysplastic syndromes and myeloproliferative diseases are myeloid in origin.

In some embodiments, a non-solid (diffuse) cancer or tumor comprises a hematopoietic malignancy, a blood cell cancer, a leukemia, a myelodysplastic syndrome, a lymphoma, a multiple myeloma (a plasma cell myeloma), an acute lymphoblastic leukemia, an acute myelogenous leukemia, a chronic myelogenous leukemia, a Hodgkin lymphoma, a non-Hodgkin lymphoma, or plasma cell leukemia.

In another embodiment, anti-IL1RAP antibodies and compositions thereof, as disclosed herein have therapeutic and/or prophylactic efficacy against diffuse cancers, for example but not limited to leukemias (e.g., acute leukemia, acute lymphocytic leukemia, acute myelocytic leukemia, acute myeloblastic leukemia, acute promyelocyte leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease.

In some embodiments, method of use treating a disease or condition treat a hematological cancer comprising leukemia, lymphoma, myeloma, acute myeloid leukemia (AML), acute promyelocytic leukemia, erythroleukemia, biphenotypic B myelomonocytic leukemia, or myelodysplastic syndromes (MDS). In some embodiments, a non-solid (diffuse cancer or tumor) comprises acute myeloid leukemia (AML). In some embodiments, a non-solid (diffuse cancer or tumor) comprises chronic myeloid leukemia (CML; also known as chronic myelogenous leukemia).

In some embodiments, the cancer or tumor comprises a metastasis of a cancer or tumor. In some embodiments, the cancer or tumor comprises a cancer or tumor resistant to other treatments.

In some embodiments of a method of treating a disease or condition, said subject is a human. In some embodiments of a method of inhibiting tumor formation or growth or both, said subject is a human.

In some embodiments, a method of treating disclosed herein reduces the minimal residual disease, increases remission, increases remission duration, reduces tumor relapse rate, prevents metastasis of the tumor or the cancer, or reduces the rate of metastasis of the tumor or the cancer, reduces the severity of the viral infection, improves the immune response to a viral infection, or any combination thereof, in the treated subject compared with a subject not administered with the anti-IL1RAP antibody or a pharmaceutical composition thereof.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present anti-IL1RAP antibodies is provided. The terms “non-human animals” and “non-human mammals” are used interchangeably herein and include all vertebrates, e.g., mammals, such as non-human primates (e.g., higher primates), sheep, dog, rodent (e.g., mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In some embodiments, methods of treating a disease or condition comprise administering a therapeutically effective amount of an anti-IL1RAP antibody or composition thereof to a subject in need. In one embodiment, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

In one embodiment, the method comprises the step of administering to the subject a composition comprising a therapeutically effective amount of the anti-IL1RAP antibody disclosed herein. In one embodiment, the composition comprises anti-IL1RAP antibodies having the heavy chain and light chain CDR sequences as described herein. In another embodiment, the composition comprises anti-IL1RAP antibodies having the VH and VL sequences as described herein.

One skilled in the art would appreciate that in some embodiments, treating a tumor or cancer encompasses a reduction of tumor size, growth, and or spread of the tumor or cancer, compared with the outcome without the use of an anti-IL1RAP antibody described herein.

In one embodiment, the present disclosure provides a method of treating a disease in a subject, comprising the step of administering to the subject a composition comprising an effective amount of the anti-IL1RAP antibody disclosed herein. In one embodiment, the composition comprises anti-IL1RAP antibodies having the heavy chain and light chain CDR sequences as described herein. In another embodiment, the composition comprises anti-IL1RAP antibodies having the VH and VL sequences as described herein.

In one embodiment, the present disclosure also provides uses of a composition comprising anti-IL1RAP antibodies for treating a disease in a subject. In one embodiment, the composition comprises anti-IL1RAP antibodies having the heavy chain and light chain CDR sequences as described herein. In another embodiment, the composition comprises anti-IL1RAP antibodies having the VH and VL sequences as described herein.

In one embodiment, the exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In one embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In another embodiment, the disease is an autoimmune disease that can be, but is not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, arthersclorosis, cardiac disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenic purpura, type 1 diabetes, ulcerative colitis, uveitis, vasculitis, and vitiligo.

In some embodiments, the disease is a transplantation-related diseases such as graft-versus-host disease (GvHD). According to one embodiment, the GVHD is acute GVHD. According to another embodiment, the GVHD is chronic GVHD.

In another embodiment, the present disclosure provides a method of using a polynucleotide to treat a disease or condition as described above, wherein the polynucleotide encodes an anti-IL1RAP antibody as described herein.

As used herein, the singular form “a”, “an” and “the” include plural references unless the context clearly dictates otherwise. For example, the term “an antibody” or “at least one antibody” may include a plurality of antibodies.

Throughout this application, various embodiments of the present disclosure may be presented in a range format. It should be understood that the description in range format is merely for convenience and brevity and should not be construed as an inflexible limitation on the scope of the anti-IL1RAP antibodies and uses thereof. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

Unless otherwise defined, all technical and/or scientific terms used herein have the same meaning as commonly understood by one of ordinary skill in the art to which the anti-IL1RAP antibodies and uses thereof pertains. Although methods and materials similar or equivalent to those described herein can be used in the practice or testing of embodiments of the anti-IL1RAP antibodies and uses thereof, methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of making and using the anti-IL1RAP antibodies and variations thereof are described. This description is not intended to be limiting and changes in the components, sequence of steps, and other variations would be understood to be within the scope of the present anti-IL1RAP antibodies and uses thereof.

It is appreciated that certain features of the anti-IL1RAP antibodies and uses thereof, which are, for clarity, described in the context of separate embodiments, may also be provided in combination in a single embodiment. Conversely, various features of the anti-IL1RAP antibodies and uses thereof, which are, for brevity, described in the context of a single embodiment, may also be provided separately or in any suitable subcombination or as suitable in any other described embodiment of the anti-IL1RAP antibodies and uses thereof. Certain features described in the context of various embodiments are not to be considered essential features of those embodiments, unless the embodiment is inoperative without those elements.

Various embodiments and aspects of the present anti-IL1RAP antibodies as delineated hereinabove and as claimed in the claims section below find experimental support in the following examples.

### EXAMPLES

### Example 1: Generation and Analysis of IL1RAP mAbs

Objective: To generate IL1RAP mAbs to human IL1RAP.

### Methods:

### Immunization, Hybridoma Generation and Antibody Recovery

The immunization was conducted using validated recombinant human ILRAP proteins (Sino Biological, extracellular domain Fc-tagged, Cat #10121-H02H and extracellular domain His-tagged, Cat #10121-H08H). A group of twelve Alivamab mice (AMM-KL; Ablexis CA USA) were immunized following the AMMPD-4 immunization protocol (ADS; https://alivamab.com/technology/) and IgG titers were assessed on Day 23 by ELISA and on Day 30 by ELISA and flow cytometry. Mice were grouped for fusion on Day 29 based on immunization strategy, strain, and titer results. Lymph nodes and spleens from immunized animals were harvested and processed into single cell suspension followed by magnetic bead-based negative enrichment for IgG secreting B cells (ADS protocol). Electrofusion was conducted on enriched lymphocyte material using NEPAgene ECFG21 instrument (NEPAgene Chiba, Japan). Hybridomas were plated into 16×384-well plates at concentration of 1×10⁶ per well and the remaining material was cryopreserved. Hybridomas were grown for 7-10 days and positive clones were expanded to 96-well plates by day 11. Finally, hybridoma cells were expanded to 40 ml volume and grown to saturation, harvested, and filtered through 0.2 μM PES membrane. Hybridoma saturated supernatants were treated with 20% MAPS II binding buffer (Biorad, CA USA; Cat #153-6161, made into 20% solution w/v) and 5M NaCl (VWR, Cat #E529) followed by filtration over 0.2 m PES. Treated supernatants were then applied to protein A resin (Amshpere A3, JSR Sciences, CA USA; Cat #BP-AMS-A3-0025) and agitated overnight at 4° C. The protein A resin beads were isolated and placed in 24-well filter plate (HTSLabs, Thomson CA, USA; Cat #921550). After wash with binding buffer, antibodies were eluted with 0.1M Citric acid and 1M NaCl, pH 3.5 (filtered through 0.2 μm PES filter) into 24-well plate (HTSLabs, Thomson CA, USA; Cat #931571) and neutralized with 1M Tris, pH 9.0 (VWR, PA, USA; Cat #E199). Eluted antibodies were concentrated to 0.3 ml volume by centrifugation at 4200 g with Vivaspin 6 columns (GE, MA, USA; Cat #28-9323-28). Concentrated antibodies were diluted in 6 ml PBS (VWR, PA, USA; Cat #0201191000) and centrifuged again at 4200 g to the final volume of 0.3 ml. After twice repeat of the last step, antibodies were diluted and sterile filtered (Santorius, Göttingen, Germany; Cat #16532-GUK). Final protein concentration was measured by Nanodrop. Next single clones were selected, and the antibodies purified by size exclusion chromatography (SEC) (See, Example 2).

Binding of IL1RAP mAbs to Human IL1RAP Protein by ELISA

Recombinant human or mouse IL1RAP proteins (Sino Biological, Beijing, China; Cat #10121-H08H and Sino Biological, Cat #52657-M08H respectively) were diluted in PBS, pH 7.4 at concentration of 1 μg/ml, coated on high-binding 384-well clear plates and incubated overnight at 4° C. Following, plates were washed 3× with 0.05% Tween-20 in PBS, pH 7.5 and then blocked with 1% BSA in PBS for 1 hour at room temperature. After 3× washes, wells were incubated with each antibody at the highest concentration of 100 nM followed by seven serial half log dilutions (in 1% BSA and 0.05% Tween-20 in PBS) for 1 hour at room temperature. Plates were then washed 3× and incubated with anti-His HRP conjugated detection antibody (Medna, TX, USA; Cat #D8212, 0.5 μg/ml diluted in 0.02% Tween-20 in PBS). After 5× washes, Supersignal ELISA Pico substrate (Thermofisher, MA, USA; Cat #37069) was applied to wells and chemiluminescent signal was read on Sprectramax L for 200 ms/well. Similarly, detection with specific κ and λ antibodies was conducted to define light chain isotyping.

Binding of mAbs to human IL1RAP protein was determined by cell-free ELISA. mAbs bound to human IL1RAP and EC50 values were calculated at the nM range.

Kinetics of IL1RAP mAbs by Octet (Creative Biolabs NY, USA)

Antibodies were diluted in kinetics buffer (0.1% BSA, 0.02% Tween-20 and 0.05% NaN₃ in PBS) and loaded onto 16 channel anti-mouse IgG Fc capture sensors (AMC, Cat #2001073; Sartorius, Göttingen, Germany). Human, huIL1RAP-His (Sino Biological, lot: MB08MA1301), and monkey, cyIL1RAP-His (Lake Pharma, NY, USA; lot:16205819371) proteins were titrated starting from the highest concentration of 100 nM and followed by 50 nM and 25 nM. Purified antibodies were loaded at 5 μg/ml in kinetics buffer. The experimental parameters selected to determine the kinetic constants were Baseline for 60 s, Loading of antibody to sensor for 180 s, Association of analyte to antibody for 120 s, Dissociation for 1200 s and Regeneration in 10 mM Glycine pH 1.7 for 4×30 s.

Antibodies demonstrated binding responses greater than 0.1 and R2 values lower than 0.9. Regeneration cycle tracking was selected for 12 cycles. Binding to human and cynomolgus IL1RAP was measured and KD (M) kinetics were calculated. Also, the disassociation rate (Kdis) (1/s) and association rate (Kon) (1/Ms) kinetics were calculated (data not shown).

### Results:

Concentrated hybridoma supernatants were analyzed for binding to human and mouse IL1RAP proteins, respectively. Eighteen (18) IL1RAP mAbs were analyzed as described in this Example and the Examples that follow, and are identified by Antibody designation and Name as follows in Table 1.

TABLE 1
IL1RAP Antibodies

| Antibody | Name |
|---|---|
| 74804_01E23A | STLX2001E |
| 74804_30O16A | STLX2030 |
| 74804_09H05A | STLX2009 |
| 74804_18L05A | STLX2018 |
| 74804_07K10A | STLX2007 |
| 74804_12A21B | STLX2012 |
| 74804_29D13A | STLX2029 |
| 74804_43F04A | STLX2043 |
| 74804_51C10A | STLX2051 |
| 74804_25H09A | STLX2025 |
| 74804_05B24A | STLX2005 |
| 74804_45C18A | STLX2045 |
| 74804_21D11A | STLX2021 |
| 74804_27I24A | STLX2027 |
| 74804_16M09A | STLX2016 |
| 74804_44J14A | STLX2044 |
| 74804_01N02A | STLX2001N |
| 74804_17E20A | STLX2017 |

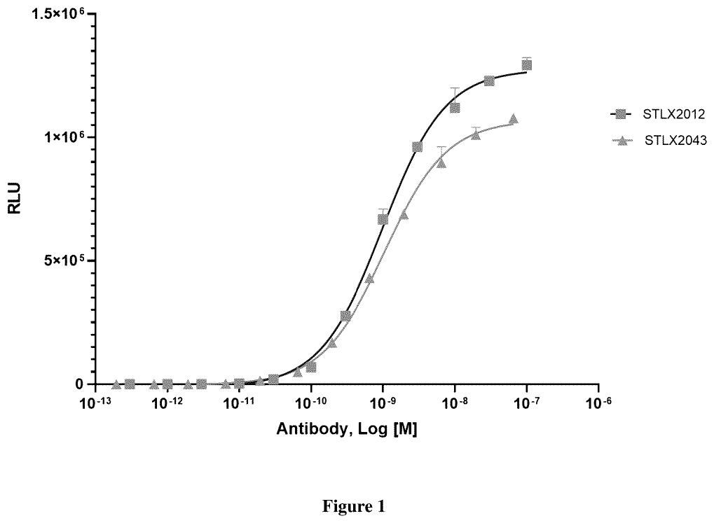

FIG. 1 shows the data from representative clones STLX2012 and STLX2043, wherein mAbs bound to human IL1RAP in a dose dependent manner. Table 2 below provides representative binding data for clones STLX2012 and STLX2043. Similar analysis was done with mouse IL1RAP, but the antibodies did not bind to mouse IL1RAP. (data not shown)

TABLE 2
Binding of mAbs to human IL1RAP

| | STLX2012 | STLX2043 |
|---|---|---|
| EC50 (M) | 9.85E−10 | 1.04E−09 |

Next, dissociation constant of mAbs to human and cynomolgus IL1RAP was determined by Octet (Creative Biolabs NY, USA). Binding affinity of each antibody is represented by Kd values that were calculated at the nM range. Table 3 shows Octet kinetics data from the purified antibodies including the binding affinity (K_{D}).

TABLE 3
Kinetic Data from purified mAbs to human and cynomolgus IL1RAP.

| | hIL1RAP | | | | | cyIL1RAP | | | | |
|---|---|---|---|---|---|---|---|---|---|---|
| Antibody | Full R\hat{ }2 | Response | KD (M) | kdis(1/s) | kon(1/Ms) | Full R\hat{ }2 | Response | KD (M) | kdis(1/s) | kon(1/Ms) |
| 74804_01E23A | 0.994 | 0.3082 | 2.34E−09 | 0.000391 | 167100 | 0.9884 | 0.2649 | 4.54E−09 | 0.000863 | 190100 |
| 74804_30O16A | 0.9776 | 0.1531 | 2.24E−09 | 0.000359 | 160500 | 0.9841 | 0.1693 | 2.59E−09 | 0.000369 | 142700 |
| 74804_09H05A | 0.9884 | 0.2052 | 1.64E−09 | 0.000272 | 165700 | 0.9861 | 0.2192 | 2.87E−09 | 0.000408 | 141800 |
| 74804_18L05A | 0.9799 | 0.1697 | 1.62E−09 | 0.00037 | 227800 | 0.9643 | 0.1684 | 4.32E−09 | 0.000954 | 221100 |
| 74804_07K10A | 0.9879 | 0.2467 | 5.96E−10 | 0.000234 | 392100 | 0.995 | 0.2726 | 7.51E−10 | 0.000283 | 376800 |
| 74804_12A21B | 0.9977 | 0.3035 | 9.34E−10 | 0.000287 | 307700 | 0.9957 | 0.3279 | 1.29E−09 | 0.000441 | 342200 |
| 74804_29D13A | 0.9968 | 0.291 | 1.07E−09 | 0.000361 | 336100 | 0.9912 | 0.3271 | 1.97E−09 | 0.00065 | 329600 |
| 74804_43F04A | 0.9919 | 0.2514 | 4.25E−10 | 0.000149 | 350200 | 0.9833 | 0.2856 | 9.7E−10 | 0.0004 | 412100 |
| 74804_51C10A | 0.9921 | 0.2375 | 8.75E−10 | 0.000301 | 343500 | 0.9954 | 0.2649 | 1.41E−09 | 0.000459 | 325600 |
| 74804_25H09A | 0.9882 | 0.2597 | 2.18E−09 | 0.000337 | 154500 | 0.9972 | 0.2633 | 2.09E−09 | 0.000297 | 142000 |
| 74804_05B24A | 0.9929 | 0.2682 | 1.67E−09 | 0.000491 | 294400 | 0.9937 | 0.3 | 2.1E−09 | 0.000563 | 267900 |
| 74804_45C18A | 0.9914 | 0.2611 | 6.48E−10 | 0.000273 | 421500 | 0.9969 | 0.2773 | 8.63E−10 | 0.000291 | 337500 |
| 74804_21D11A | 0.9926 | 0.2487 | 2.19E−09 | 0.000478 | 218200 | 0.9851 | 0.2315 | 1.02E−08 | 0.002591 | 254500 |
| 74804_27I24A | 0.9948 | 0.2288 | 2.13E−09 | 0.000747 | 350400 | 0.9921 | 0.2419 | 2.82E−09 | 0.001071 | 379500 |
| 74804_16M09A | 0.9903 | 0.2568 | 8.34E−10 | 0.000411 | 493400 | 0.8141 | 0.1991 | 3.94E−09 | 0.003996 | 1014000 |
| 74804_44J14A | 0.9905 | 0.2683 | 1.23E−09 | 0.000684 | 558300 | 0.7075 | 0.2225 | 1.68E−09 | 0.002061 | 1226000 |
| 74804_01N02A | 0.99 | 0.3084 | 8.03E−10 | 0.000233 | 289800 | 0.9948 | 0.3424 | 1.23E−09 | 0.000413 | 334300 |
| 74804_17E20A | 0.9846 | 0.17 | 8.3E−10 | 0.000348 | 419500 | 0.9926 | 0.1922 | 7.39E−10 | 0.000259 | 350800 |

Summary: 18 mAb clones were identified, and their showed affinity for human IL1RAP in the nM range.

### Example 2: Purification of IL1RAP mAbs

Objective: To purify the IL1RAP mAbs.

### Methods:

Size-Exclusion (SEC) mAbs Analysis

SEC-HPLC column (Tosoh Tokyo, Japan; TGKgel SuperSW3000 4.6 mm ID×300 mm, 4 μM PN:18675) was equilibrated in isocratic running buffer (0.1M Na₂PO₄ and 0.1M NaSO₄, pH 6.7, filtered through 0.2 μM PES membrane) at flow rate of 0.35 ml/min. Known protein standards were injected as controls with refrigerated autosampler and 40 μl injection loop onto TSKgel SuperSW3000 (TOSOH, Cat #18675), followed by loading of each antibody at the concentration of 10 μg. Absorbance was measured at 280 nm, 254 nm, and 215 nm via diode array detector and the % main peak was calculated by AUC in Chemstation (Ohio, USA) software.

### Antibody Assessment by SDS-PAGE

Antibodies were analyzed under non-reducing and reducing conditions. For non-reducing conditions antibodies (2 μg) were mixed with 4× loading buffer (Expedeon Cambridge, England; PN: NXB31010 LN:19A08003) containing N-ethyl maleimide (1 μl), while for reducing conditions they were mixed with 4× loading buffer containing DTT (1 μl of 1M). Samples prepared in reducing conditions were also boiled at 95° C. for 5 minutes. All samples and standards (Biorad, cat: 160375) were loaded onto gels (Expedeon, Cat #NXG42012) and run at constant 200V for 50 minutes. Finally, gels were washed for 1 minute with water and stained with InstantBlue (Expedeon, Cat #ISB1L). Gels were imaged with Azure Biosystems c200 in the visible setting on an orange plate with automatic exposure on.

### Results:

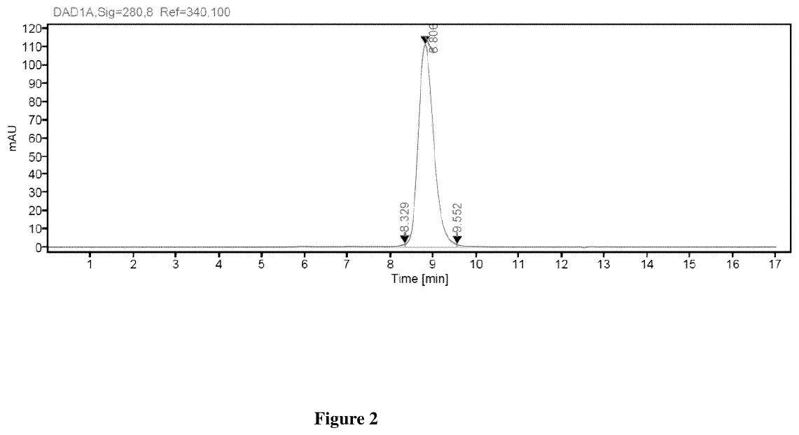

Eighteen (18) antibodies were profiled by size-exclusion analysis conducted in SEC-HPLC. All antibodies showed a >96% symmetrical main peak with retention time typical for intact IgG monomer (Data not shown) FIG. 2 shows representative results of the SEC analysis for one of the clones, STLX2043.

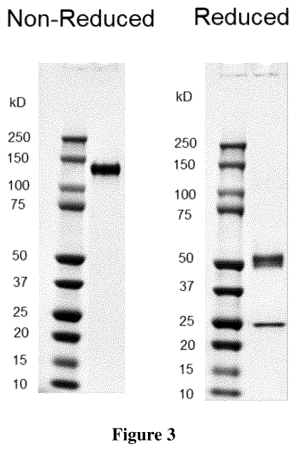

Next, antibodies were analyzed under non-reducing and reducing conditions. FIG. 3 provides representative data showing expression analysis of IL1RAP mAb STLX2043. Under non-reducing conditions a band migrating at approximately 150 kDa, as typical for IgG, was detected in all antibody samples (data not shown). Under reducing conditions, a band migrating at 50 kDa, as typical of IgG heavy chain and a band migrating at 25 kDa, as typical of IgG light chain, were detected in all antibody samples (data not shown).

IL1RAP mAbs were sequenced after purification and after the reporter assays; wherein the associated VH, VL, and CDR amino acid sequence reference numbers are presented below in Tables 3 and 4.

### Tables 4A-4C: Amino Acid Sequences and SEQ ID NPs: of Variable Heavy (VH) and Variable Light (VL) Regions of IL1RAP Antibodies

TABLE 4A
SEQ ID NOs: and Light Chain type for IL1RAP Antibodies.

| | | Light | |
|---|---|---|---|
| | | Chain | |
| | VH Region | (LC) | VL Region |
| Antibody | SEQ ID NO: | Type | SEQ ID NO: |
| 74804_01E23A | 1 | κ | 18 |
| 74804_30O16A | 2 | κ | 19 |
| 74804_09H05A | 3 | κ | 20 |
| 74804_18L05A | 4 | κ | 21 |
| 74804_07K10A | 5 | κ | 22 |
| 74804_12A21B | 6 | κ | 23 |
| 74804_29D13A | 6 | κ | 22 |
| 74804_43F04A | 7 | κ | 22 |
| 74804_51C10A | 8 | κ | 24 |
| 74804_25H09A | 9 | κ | 25 |
| 74804_05B24A | 10 | κ | 26 |
| 74804_45C18A | 11 | κ | 27 |
| 74804_21D11A | 12 | λ | 28 |
| 74804_27I24A | 13 | λ | 29 |
| 74804_16M09A | 14 | λ | 30 |
| 74804_44J14A | 15 | λ | 31 |
| 74804_01N02A | 16 | κ | 18 |
| 74804_17E20A | 17 | κ | 32 |

TABLE 4B
Amino acid Sequences & SEQ ID NOs: of Variable Heavy (VH)
Region of IL1RAP Antibodies.

| | | SEQ ID |
|---|---|---|
| Antibody | VH Region | NO: |
| 74804_01 | QVQLVQSGAEVKKPGASVKVSCKASGYTFTGYYMHWVRQAPGQGLEWMGWLNPNSGGTDYV | 1 |
| E23A | QKFQGRVTMTRDTSISTAYMELSRLRSDDTAVYYCARTLYYYGMDVWGQGTTVTVSS | |
| 74804_30 | QVQLVQSGAEVKRPGASVKVSCKASGYTFTGYYMHWVRQAPGQGLEWMGWINPYSGGTDYA | 2 |
| O16A | QKFQDRVTMTRDTSISTAYMELSRLRSDDTAVYYCARSYYYYGMDVWGQGITVTVSS | |
| 74804_09 | QVQLFQSGAEVKKPGASVKVSCKASGYTFTGYYVHWVRQAPGQGLEWMGWINPNSGGTDYA | 3 |
| H05A | QKFQGRVTMTWDTSISTAYMELSRLRSDDTAVYYCARSYYYYGMDVWGQGTTVTVSS | |
| 74804_18 | QVQLVQSGAEVKKPGASVKVSCKTSGYTFTSCCMHWVRQAPGQGLEWMGIINPSVGSTSYA | 4 |
| L05A | QKFQGIVTMTRDTSTSTVYMELSSLRSEDTAVYYCAVDSSGSRYYYGMDVWGQGTTVTVSS | |
| 74804_07 | QVQLLQSGAEVKKPGSSVKVSCRASGGTFSIYAIDWVRQAPGQGLEWMGGIIPISGTENSA | 5 |
| K10A | QNFQDRITITADKSTNTAYMELSSLRSEDTAVYYCARNGATSDAFDIWGQGTMVTVSS | |
| 74804_12 | QVQLLQSGAEVKKPGSSVKVSCKASGGTFSIYAIDWVRQAPGQGLEWMGGIIPIFGTANSA | 6 |
| A21B | QKFQGRVTITADKSTSTAYMELSSLRSEDTAVYYCARNGATSDAFDMWGQGTMVTVSS | |
| 74804_29 | QVQLLQSGAEVKKPGSSVKVSCKASGGTFSIYAIDWVRQAPGQGLEWMGGIIPIFGTANSA | 6 |
| D13A | QKFQGRVTITADKSTSTAYMELSSLRSEDTAVYYCARNGATSDAFDMWGQGTMVTVSS | |
| 74804_43 | QVQLLQSGAEVKKPGSSVKVSCKASGGTFSIYAIDWVRQAPGQGLEWMGGIIPIFGTENSA | 7 |
| F04A | QNFQGRITITADKSTNTAYMELSSLRSEDTAVYYCARNGATSDAFDIWGQGTMVTVFS | |
| 74804_51 | QVQLLQSGAEVKKPGSSVKVSCKASGGTFNIYAIDWVRQAPGQGLEWMGGIIPIFGTANSA | 8 |
| C10A | QKFQGRVTITADKSTNTAYMELSSLRSEDTAVYYCARNGATSDAFDIWGQGTMVTVSS | |
| 74804_25 | QVQLVQSGAEVKKPGSSVKVSCKASGDTFSNYAITWVRQAPGQGLEWIVGFSPIFGTANYA | 9 |
| H09A | QKFQGRVTITADKSTSTAYMELSSLISEDTAVYYCAWGSGNFNWFDPWGQGTLVTVSS | |
| 74804_05 | EVQLVDSGGGLVKPGGSLRLSCAATGFTFSNVWMSWVRQGSGKGLEWVGRIKSKTDGGTID | 10 |
| B24A | YAAPVKGRFTISRDDPKNTLYLQMNSLKTEDTAVYYCTTGELLGFDYWGQGTLVTVSS | |
| 74804_45 | EVQLVESGGGLVKPGGSLRLACAATGFTFSNVWMSWVRQGSGKGLEWVGRIKSKADGGTID | 11 |
| C18A | YAAPVKGRFTISRDDSKNTLYLQMNSLKTEDTAMYYCTTGELLGFDYWGQGTLVTVSS | |
| 74804_21 | EVQVVESGGGLVKPGGSLRLSCAASGFIFSKAWMSWVRQAPGKGLEWVGRIKSKTDGGTTD | 12 |
| D11A | YAAPVKGRFTISRDDSKNTLYLQMNSLKTEDTAVYYCTTGGYVFDYWGQGALVTVSS | |
| 74804_27 | EVQLVESGGGLVKPGGSLRLSCAASGFTFITYSMNWVRQAPGKGLEWVSSISSISSYIYYA | 13 |
| I24A | DSVKGRFTISRDNAKNSLYLQMDSLRAEDSAVYYCAREGIVGPTGYFDSWGQGTLVTVSS | |
| 74804_16 | QVQLQESGPGLVKPSETLSLTCTVSGGSISSYFWNWIRQPPGKGLEWIGYGHYSGNSNHNP | 14 |
| M09A | SLKSRVTISVDMSKNQFSLKMSSVTAADTAMYYCAREGLHDAFDIWGQGTMVTVSS | |
| 74804_44 | QVQLQESGPGLVKPSETLSLTCTVSGGSVFSYFWNWIRQPPVKGLEWIGYIDHSGGTNYNP | 15 |
| J14A | SLKSRVTMSVDTSKNQFSLKLSSVTAADTAVYYCAREGLHDAFDIWGQGTLVTVSS | |
| 74804_01 | EVQLVQSGAEVKKPGESLKISCKGSGYSFTSYWIVWVRQMPGKGLEWMGFIYPGNSDTRYS | 16 |
| N02A | PSFQGQVTISADQSISTAYLQWSSLKASDTATYYCARGGSYYLDYWGQGTLVTVSS | |
| 74804_17 | EVQLVQSGAEVKKPGESLKISCKGSGYSFTSYWIGWVRQMPGKGLEWMGIIWPGDSDTRYS | 17 |
| E20A | PSFQGQVTISADKSVNTAYLQWISLKASDTAIYYCARSSGGTAMDVWGQGTTVTVSS | |

TABLE 4C
Amino acid Sequences & SEQ ID NOs: of Variable Light (VL)
Region and Type of IL1RAP Antibodies.

| Anti- | | | SEQ ID |
|---|---|---|---|
| body | Type | VL Region | NO: |
| 74804_ | κ | DIVMTQSPDSLAVSLGERATIYCKSSQSVLINSNNQNYLAWYQQKPGQPPKLLI | 18 |
| 01E23A | | QWASTRESGVPDRFSGSGSGTDFTLTISSLQAEDVAVYYCQQYYNSPFTFGGGT | |
| | | KVEIK | |
| 74804_ | κ | DIVMTQSPDSLAVSLGERATINCKSSQNIINSSTNKNYLAWYKQKPGQPPKLLI | 19 |
| 30016A | | YWASTRESGVPDRFSGSGSGTDFTLTISSLQAEDVAVYYCQQYYSTPFTFGGGT | |
| | | KVEIK | |
| 74804_ | κ | DIVMTQSPDSLAVSLGERATINCKSSQSVLYSSNNTSYLAWYQQKPGQPPKFLI | 20 |
| 09H05A | | YWTSTRESGVPDRFSGSGSGTDFTLTISSLQAEDVAVYYCQQYYITPFTFGPGT | |
| | | KVDIK | |
| 74804_ | κ | EIVMTQSPATLSVSPGERATLSCRASQSVRSNLAWYQQRPGQAPRLLIYGTSTR | 21 |
| 18L05A | | ATGIPARFSGSGSGTEFTLTISSLQSEDFAVYYCQQYNYWPYTFGQGTKLEIK | |
| 74804_ | κ | EIVLTQSPGTLSLSPGERATLSCRASLSVSSNYLAWFQQRPGQAPRLLIYGVSR | 22 |
| 07K10A | | RATGIPDRFSGSGSGTDFTLTISRLEPEDFAVYYCQQYGSSPLTFGGGTKVEIK | |
| 74804_ | κ | EIVLTQSPGTLSLSPGERATLSCRASLSVSSNYLAWFQQRPGQAPRLLIHGVSR | 23 |
| 12A21B | | RATGIPDRFSGSGSGTDFTLTISRLEPEDFAVYYCQQYGSSPLTFGGGTKVEIK | |
| 74804_ | κ | EIVLTQSPGTLSLSPGERATLSCRASLSVSSNYLAWFQQRPGQAPRLLIYGVSR | 22 |
| 29D13A | | RATGIPDRFSGSGSGTDFTLTISRLEPEDFAVYYCQQYGSSPLTFGGGTKVEIK | |
| 74804_ | κ | EIVLTQSPGTLSLSPGERATLSCRASLSVSSNYLAWFQQRPGQAPRLLIYGVSR | 22 |
| 43F04A | | RATGIPDRFSGSGSGTDFTLTISRLEPEDFAVYYCQQYGSSPLTFGGGTKVEIK | |
| 74804_ | κ | EIVLTQSPGTLSLSPGERATLSCRASQSVSSNYLAWFQQRPGQAPRLLIYGVSR | 24 |
| 51C10A | | RATGIPDRFSGSGSGTDFTLTISRLEPEDFAVYYCQQYGSSPLTFGGGTKVEIK | |
| 74804_ | κ | AIRMTQSPSSFSASTGDRVTITCRASQGISSYLAWYQQKPGKAPKLLIYAASTL | 25 |
| 25H09A | | SQGVPSRFSGSGSGTDFTLTISSLQSEDFATYYCQQYYSYPLTFGGGTKVEIK | |
| 74804_ | κ | DIVMTQSPLSLPVTPGEPASISCRSGQSLLHNNGFNCLAWYLQKPGQSPQLLIY | 26 |
| 05B24A | | LGSNRASGVPDRFSGSGSGTDFTLKISRVEAEDVGVFYCMQTLQTPITFGQGTR | |
| | | LEIK | |
| 74804_ | κ | DIVMTQSPLSLPVTPGEPASISCRSGQSLLHSNGFNCLAWYLQKPGQSPQLLIY | 27 |
| 45C18A | | LGSNRASGVPDRFSGSGSGTDFTLKISRVEAEDVGVYYCMQTLQTPITFGQGTR | |
| | | LEIK | |
| 74804_ | λ | QSVLTQPPSASGTPGQRVTISCSGNNSNIGSYIVNWFQQLPGTAPKLLIYSKNQ | 28 |
| 21D11A | | RPSGVPDRFSGSKSGTSASLAISGLQSEDEADYYCAAWDDSLNGVVFGGGTKLT | |
| | | VL | |
| 74804_ | λ | QSVLTQPPSVSAAPGQKVTISCSGSSSNIGNHYVSWYQQLPGTAPKLLIYDNNK | 29 |
| 27I24A | | RPSGIPDRFSGSKSGTSATLGITGLQTGDEADYYCGTWDSRLSAYWVFGGGTKL | |
| | | TVL | |
| 74804_ | λ | QSVLTQPPSASGTPGQRVTISCSGSISNIGSNTVNWYQQLPGTAPKLLIYSNNQ | 30 |
| 16M09A | | RPSGVPDRFSGSKSGTSASLAISGLQSEDEADYYCAAWDDSLNGRVFGGGTKLT | |
| | | VL | |
| 74804_ | λ | QSVLTQPPSASGTPGQRVTISCSGSSSNIGSNTVNWYQLLPGTAPKLLIYSNYQ | 31 |
| 44J14A | | RPSGVPDRFSGSKSGTSASLAISGLQSEDEADYYCAAWDDSLNGRVFGGGTKLT | |
| | | VL | |
| 74804_ | κ | DIVMTQSPDSLAVSLGERATIYCKSSQSVLINSNNQNYLAWYQQKPGQPPKLLI | 18 |
| 01N02A | | QWASTRESGVPDRFSGSGSGTDFTLTISSLQAEDVAVYYCQQYYNSPFTFGGGT | |
| | | KVEIK | |
| 74804_ | κ | DIVMTQSPLSLPVTPGEPASISCRSSQSLLHSNGYNTLDWYLQKPGQSPQLLIY | 32 |
| 17E20A | | LGSNRASGVPDRFSGSGSGTDFTLKISRVEAEDVGLYYCMQGLQTQWTFGQGTR | |
| | | VEIK | |

### Tables 5A-5C: SEQ ID NOs: and Amino Acid Sequences of CDR Regions of IL1RAP Antibodies

TABLE 5A
SEQ ID NOs: of HCDR1-3 and LCDR1-3 of IL1RAP Antibodies.

| | HCDR1 | HCDR2 | HCDR3 | LCDR1 | LCDR2 | LCDR3 |
|---|---|---|---|---|---|---|
| | SEQ ID | SEQ ID | SEQ ID | SEQ ID | SEQ ID | SEQ ID |
| Antibody | NO: | NO: | NO: | NO: | NO: | NO: |
| 74804_01E23A | 33 | 45 | 61 | 73 | 87 | 98 |
| 74804_30O16A | 33 | 46 | 62 | 74 | 87 | 99 |
| 74804_09H05A | 33 | 47 | 62 | 75 | 88 | 100 |
| 74804_18L05A | 34 | 48 | 63 | 76 | 89 | 101 |
| 74804_07K10A | 35 | 49 | 64 | 77 | 90 | 102 |
| 74804_12A21B | 35 | 50 | 65 | 77 | 90 | 102 |
| 74804_29D13A | 35 | 50 | 65 | 77 | 90 | 102 |
| 74804_43F04A | 35 | 51 | 64 | 77 | 90 | 102 |
| 74804_51C10A | 36 | 50 | 64 | 78 | 90 | 102 |
| 74804_25H09A | 37 | 52 | 66 | 79 | 91 | 103 |
| 74804_05B24A | 38 | 53 | 67 | 80 | 92 | 104 |
| 74804_45C18A | 39 | 54 | 67 | 81 | 92 | 104 |
| 74804_21D11A | 40 | 55 | 68 | 82 | 93 | 105 |
| 74804_27I24A | 41 | 56 | 69 | 83 | 94 | 106 |
| 74804_16M09A | 42 | 57 | 70 | 84 | 95 | 107 |
| 74804_44J14A | 43 | 58 | 70 | 85 | 96 | 107 |
| 74804_01N02A | 44 | 59 | 71 | 73 | 87 | 98 |
| 74804_17E20A | 44 | 60 | 72 | 86 | 97 | 108 |

TABLE 5B
Amino Acid Sequences & SEQ ID NOs: of HCDR1, HCDR2,
HCDR3 of IL1RAP Antibodies.

| | | SEQ ID | | SEQ ID | | SEQ ID |
|---|---|---|---|---|---|---|
| Antibody | HCDR1 | NO: | HCDR2 | NO: | HCDR3 | NO: |
| 74804_01E23A | GYTFTGYY | 33 | LNPNSGGT | 45 | ARTLYYYGMDV | 61 |
| 74804_30016A | GYTFTGYY | 33 | INPYSGGT | 46 | ARSYYYYGMDV | 62 |
| 74804_09H05A | GYTFTGYY | 33 | INPNSGGT | 47 | ARSYYYYGMDV | 62 |
| 74804_18L05A | GYTFTSCC | 34 | INPSVGST | 48 | AVDSSGSRYYYGMDV | 63 |
| 74804_07K10A | GGTFSIYA | 35 | IIPISGTE | 49 | ARNGATSDAFDI | 64 |
| 74804_12A21B | GGTFSIYA | 35 | IIPIFGTA | 50 | ARNGATSDAFDM | 65 |
| 74804_29D13A | GGTFSIYA | 35 | IIPIFGTA | 50 | ARNGATSDAFDM | 65 |
| 74804_43F04A | GGTFSIYA | 35 | IIPIFGTE | 51 | ARNGATSDAFDI | 64 |
| 74804_51C10A | GGTFNIYA | 36 | IIPIFGTA | 50 | ARNGATSDAFDI | 64 |
| 74804_25H09A | GDTFSNYA | 37 | FSPIFGTA | 52 | AWGSGNFNWFDP | 66 |
| 74804_05B24A | GFTFSNVW | 38 | IKSKTDGGTI | 53 | TTGELLGFDY | 67 |
| 74804_45C18A | GFTFSNVM | 39 | IKSKADGGTI | 54 | TTGELLGFDY | 67 |
| 74804_21D11A | GFIFSKAW | 40 | IKSKTDGGTT | 55 | TTGGYVFDY | 68 |
| 74804_27124A | GFTFITYS | 41 | ISSISSYI | 56 | AREGIVGPTGYFDS | 69 |
| 74804_16M09A | GGSISSYF | 42 | GHYSGNS | 57 | AREGLHDAFDI | 70 |
| 74804_44J14A | GGSVFSYF | 43 | IDHSGGT | 58 | AREGLHDAFDI | 70 |
| 74804_01N02A | GYSFTSYW | 44 | IYPGNSDT | 59 | ARGGSYYLDY | 71 |
| 74804_17E20A | GYSFTSYW | 44 | IWPGDSDT | 60 | ARSSGGTAMDV | 72 |

TABLE 5C
Amino Acid Sequences & SEQ ID NOs: of LCDR1, LCDR2,
LCDR3 of IL1RAP Antibodies.

| | | SEQ ID | | SEQ ID | | SEQ ID |
|---|---|---|---|---|---|---|
| Antibody | LCDR1 | NO: | LCDR2 | NO: | LCDR3 | NO: |
| 74804_01E23A | QSVLINSNNQNY | 73 | WAS | 87 | QQYYNSPFT | 98 |
| 74804_30016A | QNIINSSTNKNY | 74 | WAS | 87 | QQYYSTPFT | 99 |
| 74804_09H05A | QSVLYSSNNTSY | 75 | WTS | 88 | QQYYITPFT | 100 |
| 74804_18L05A | QSVRSN | 76 | GTS | 89 | QQYNYWPYT | 101 |
| 74804_07K10A | LSVSSNY | 77 | GVS | 90 | QQYGSSPLT | 102 |
| 74804_12A21B | LSVSSNY | 77 | GVS | 90 | QQYGSSPLT | 102 |
| 74804_29D13A | LSVSSNY | 77 | GVS | 90 | QQYGSSPLT | 102 |
| 74804_43F04A | LSVSSNY | 77 | GVS | 90 | QQYGSSPLT | 102 |
| 74804_51C10A | QSVSSNY | 78 | GVS | 90 | QQYGSSPLT | 102 |
| 74804_25H09A | QGISSY | 79 | AAS | 91 | QQYYSYPLT | 103 |
| 74804_05B24A | QSLLHNNGFNCL | 80 | GSN | 92 | MQTLQTPIT | 104 |
| 74804_45C18A | QSLLHSNGFNCL | 81 | GSN | 92 | MQTLQTPIT | 104 |
| 74804_21D11A | NSNIGSYI | 82 | SKN | 93 | AAWDDSLNGVV | 105 |
| 74804_27124A | SSNIGNHY | 83 | DNN | 94 | GTWDSRLSAYWV | 106 |
| 74804_16M09A | ISNIGSNT | 84 | SNN | 95 | AAWDDSLNGRV | 107 |
| 74804_44J14A | SSNIGSNT | 85 | SNY | 96 | AAWDDSLNGRV | 107 |
| 74804_01N02A | QSVLINSNNQNY | 73 | WAS | 87 | QQYYNSPFT | 98 |
| 74804_17E20A | QSLLHSNGYNT | 86 | LGS | 97 | MQGLQTQWT | 108 |

Summary: 18 mAbs were isolated, purified, and sequenced to identify variable heavy and light chain amino acid sequences, as well as the CDR amino acid sequences.

### Example 3: Blocking of IL1R1/IL1B/IL1RAP Complex Formation and Suppression of IL1-Induced NFκB Activity by IL1RAP mAbs

Objective: IL1RAP associates with IL1R1 bound to IL1B to form the high affinity interleukin-1 receptor complex, which mediates interleukin-1-dependent activation of NF-kappa-B and other pathways. The mAbs were assayed for their ability to block the formation of the IL1R1/IL1B/IL1RAP complex, and to determine the downstream effect on NFκB activity. IL1RAP is also a coreceptor for IL1RL1 in the IL-33 signaling system. Therefore, analysis of mAb suppression of IL33-induced NFκB activity was also assessed for the mAb IL1RAP antibodies.

### Methods:

Blocking of IL1R1/IL1B/IL1RAP Complex Formation by IL1RAP mAbs

High-binding 96-well plates were coated with IL1RAP-Fc protein (Sino Biological, Cat #10121-H02H) at concentration of 4 g/ml and incubated overnight at 4° C. After 3× washes with 0.05% Tween-20 in PBS, pH 7.5, plates were blocked with 1% BSA in PBS for 1 hour at room temperature. Following, wells were incubated with each antibody at concentration of 50 nM for 1 hour at room temperature. Plates were then washed 3× and wells were incubated with premixed complex of IL1R1-His protein (Sino Biological, Cat #10126-H08H) at concentration of 1 g/ml and IL1β cytokine (Sino Biological, Cat #10139-HNAE) at concentration of 0.5 g/ml for 1 hour at room temperature. IL1R1/IL1β complex was preincubated in assay buffer (1% BSA in PBS, pH 7.5) for 20 min at room temperature. After 3× washes, plates were incubated with anti-His HRP detection antibody (Medna, Cat #D8212, 0.5 g/ml) for 1 hour at room temperature. Following 5× washes, SuperPico chemiluminescent substrate was applied to wells and signal was read on Spectramax M5.

Suppression of IL1-Induced NFκB Activity by IL1RAP mAbs

The HEK-Blue IL1β (Invivogen, Cat #hkb-il1b) cell line was used to monitor NFκB activity upon stimulation with IL1β cytokine. In this cell line the secreted embryonic alkaline phosphatase reporter gene (SEAP) is inducibly expressed under the control of IFN-β minimal promoter fused to NFκB/AP-1. Specificity to IL1β cytokine was achieved by blockage of TNF-α response. The cells were maintained in the presence of hygromycin (200 μg/ml) and zeocin (100 μg/ml).

For the functional assay, cells were plated at a density of 5×10⁴ cells/well in 96-well plates. Cells were treated with each antibody at the highest concentration of 200 μg/ml followed by seven serial 1:3 dilutions and incubated for 45 minutes at 37° C./5% CO₂ in humified incubator. Cells were then stimulated with IL1β (12 μM) and incubated overnight at 37° C./5% CO₂ in humified incubator. Every treatment was conducted in triplicates. Following, supernatants from each well were mixed with Quanti-Blue substrate (Invivogen, CA USA; Cat #rep-qb1) according to manufacturer's recommendations and secreted SEAP was measured using spectrophotometer at 650 nm.

Suppression of IL33-Induced NFκB Activity by IL1RAP mAbs

HEK-Blue IL33 (Invivogen, Cat #hkb-hil33) cell line was used to monitor NFκB activity upon stimulation with IL33 cytokine. The secreted embryonic alkaline phosphatase reporter gene (SEAP) is inducible expressed under the control of IFN-β minimal promoter fused to NFκB/AP-1. HEK-BlueIL33 cells were also generated upon stable transfection with human IL1RL1 (ST2) gene. Specificity to IL33 cytokine was achieved by blockage of TNF-α and IL1 response. Cells are maintained in the presence of hygromycin (200 μg/ml), zeocin (100 μg/ml) and blasticidin (10 μg/ml).

For the functional assay, cells were plated at a density of 5×10⁴ cells/well in 96-well plates. Cells were treated with each antibody at the highest concentration of 200 μg/ml followed by seven serial 1:3 dilutions and incubated for 45 minutes at 37° C./5% CO² in humified incubator. Cells were then stimulated with IL33 (12 μM) and incubated overnight at 37° C./5% CO₂ in humified incubator. Every treatment was conducted in triplicates. Following, supernatants from each well were mixed with Quanti-Blue substrate (Invivogen, Cat #rep-qb1) according to manufacturer's recommendations and secreted SEAP was measured using spectrophotometer at 650 nm.

### Results:

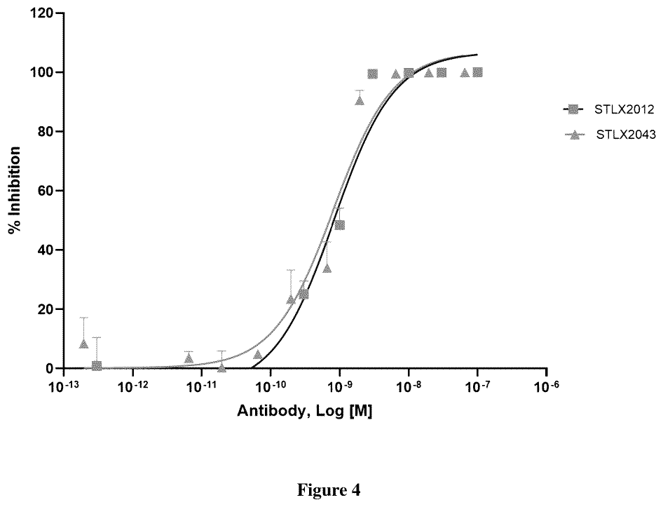

Blocking of IL1R1/IL1β/IL1RAP complex formation by mAbs was measured by cell-free ELISA. FIG. 4 shows representative mAbs (STLX2012 and STLX2043) block IL1R1/IL1β/IL1RAP complex formation in a dose dependent manner, wherein and IC50 values were calculated at the nM range. (Table 6)

TABLE 6
Blocking of IL1R1/IL1β/IL1RAP
complex formation by IL1RAP mAbs.

| | STLX2012 | STLX2043 |
|---|---|---|
| IC50 (M) | 8.235E−10 | 8.041E−10 |

Similar results were observed for the other anti IL1RAP mAbs (Data not shown).

FIGS. 5A and 5B show that all 18 IL1RAP mAbs inhibited WI-induced NFκB activity in a dose-dependent manner. Inhibition was measured by the HEK-BlueIL1β cell-based reporter assay. Table 7 below provides the IC50s, which were calculated at the nM range, and provides the % inhibition at 200 nM, which surprisingly is at or extremely near 100%.

TABLE 7
Inhibition of IL1-induced NFκB activity by IL1RAP mAbs.
HEK-BlueIL1β NFκB Reporter Assay

| | | Max % |
|---|---|---|
| | | Inhibition |
| Antibody | IC50 | at 200 nM |
| 74804_01E23A | 7.37E−10 | 98 |
| 74804_30O16A | 2.31E−09 | 98 |
| 74804_09H05A | 2.00E−09 | 99 |
| 74804_18L05A | 1.73E−09 | 99 |
| 74804_07K10A | 3.19E−09 | 100 |
| 74804_12A21B | 1.31E−09 | 100 |
| 74804_29D13A | 1.85E−09 | 99 |
| 74804_43F04A | 2.29E−09 | 99 |
| 74804_51C10A | 2.50E−09 | 99 |
| 74804_25H09A | 1.77E−09 | 100 |
| 74804_05B24A | 4.46E−10 | 99 |
| 74804_45C18A | 1.93E−10 | 100 |
| 74804_21D11A | 5.32E−10 | 101 |
| 74804_27I24A | 1.96E−09 | 100 |
| 74804_16M09A | 8.05E−10 | 100 |
| 74804_44J14A | 7.92E−10 | 98 |
| 74804_01N02A | 1.72E−09 | 101 |
| 74804_17E20A | 8.19E−10 | 101 |

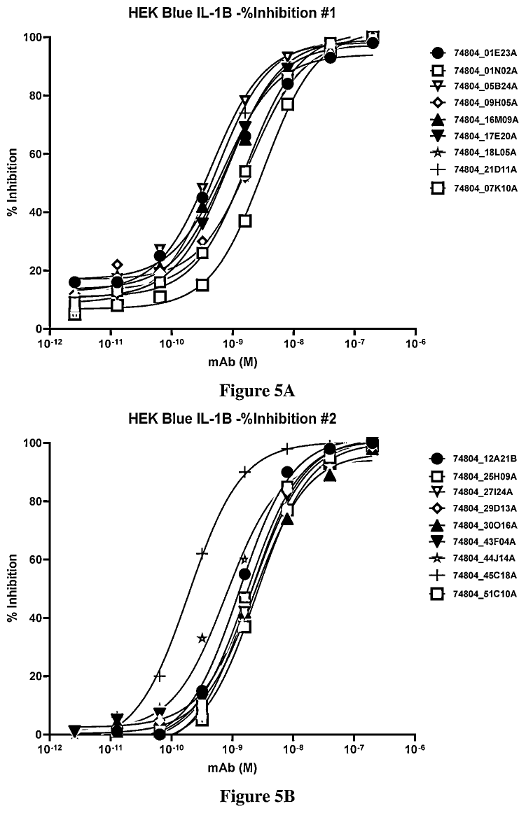

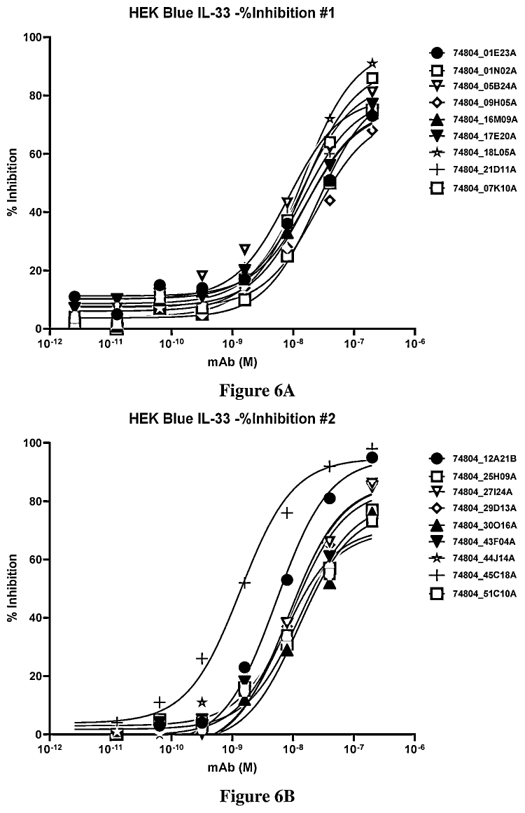

FIGS. 6A and 6B show that all of the IL1RAP mAb clones also inhibited IL33-induced NFκB activity in a dose-dependent manner. Inhibition was measured by the HEKBlue-IL33 cell-based reporter assay. Table 8 below provides the IC50s, which were calculated in the nM range, and provides the % inhibition at 200 nM

TABLE 8
Inhibition of IL33-induced NFκB activity by IL1RAP mAbs.
HEK-BlueIL33 NFκB Reporter Assay

| | | Max % |
|---|---|---|
| | | Inhibition |
| Antibody | IC50 | at 200 nM |
| 74804_01E23A | 2.49E−09 | 80 |
| 74804_30O16A | 1.24E−08 | 76 |
| 74804_09H05A | 1.10E−08 | 74 |
| 74804_18L05A | 4.81E−09 | 93 |
| 74804_07K10A | 6.62E−09 | 79 |
| 74804_12A21B | 5.316−09 | 95 |
| 74804_29D13A | 8.75E−09 | 86 |
| 74804_43F04A | 9.62E−09 | 85 |
| 74804_51C10A | 1.30E−08 | 77 |
| 74804_25H09A | 6.55E−09 | 73 |
| 74804_05B24A | 2.72E−09 | 85 |
| 74804_45C18A | 1.37E−09 | 98 |
| 74804_21D11A | 3.54E−09 | 85 |
| 74804_27I24A | 8.31E−09 | 86 |
| 74804_16M09A | 7.66E−09 | 76 |
| 74804_44J14A | 8.12E−09 | 73 |
| 74804_01N02A | 1.70E−08 | 87 |
| 74804_17E20A | 4.82E−09 | 80 |

Summary: The results shown here demonstrate that the IL1RAP mAbs blocked IL1R1/IL1B/IL1RAP complex formation and suppressed downstream NFκB activities in a dose-dependent manner.

### Example 4: Inhibition of IL1, IL33 and IL36-Induced Signaling in AML Patient Samples or Cancer Cells by IL1RAP mAbs

Objective: To determine if the IL1RAP mAbs were able to inhibit IL1, IL33 and IL36-induced signaling in acute myeloid leukemia (AML) patient samples or different types of cancer cells.

### Methods:

AML patient samples or cancer cells (AML, chronic myelogenous leukemia (CML), pancreatic, head and neck squamous cell carcinoma (HNSCC), bladder, non-small-cell lung carcinoma (NSCLC), colorectal and triple-negative breast cancer (TNBC)) were seeded in 6-well plates (5×10⁵ cells/well) and incubated overnight at 37° C./5% CO₂ in humified incubator. In experiments conducted in cancer cell lines, cells were washed 2× with PBS and serum-starved for 3 hours. Following, cells were treated with IL1RAP antibodies at concentration of 20 μg/ml and incubated for 1 h at 37° C./5% CO₂ in humidified incubator. After 1 h, cells were treated with IL1β (Invivogen, cat: rcyec-hil1b), IL33 (Adipogen, cat: AG-40B-0160-C010) or IL36 cytokines (R@D, cat: 6836IL) at 100 ng/ml and incubated for 15 more minutes. Cells were harvested on ice after 1× wash with cold PBS and were centrifuged for 5 minutes at 5000 rpm at 4° C. Cells were then lysed with NP-40 buffer for 5 minutes on ice and centrifuged for 15 minutes at 15000 rpm at 4° C. Supernatants were collected and protein concentrations were determined by BCA (Thermofisher, Cat #PI23225). Supernatants were mixed with 4× sample buffer and boiled for 5 minutes at 95° C. Following, equal amounts of proteins (30 g per lane) were loaded to gels (Thermofisher, Cat #NP0335BOX or Cat #NP0323BOX) and run at 100V for 2 hours. Proteins were transferred to nitrocellulose membranes (Amersham, Cat #10600006) for 1.5 hour at 100V. Membranes were then blocked in 5% non-fat dry milk and 0.1% Tween-20 in TBS for 30 minutes at room temperature. Following, membranes were incubated with indicated primary antibodies (Cell signaling technology, phospho-ERK Cat #9101, phospho-AKT Cat #9271, phospho-p38 Cat #9211, phospho-NFκB Cat #3033, and β-Actin Cat #4970) in 5% BSA and 0.1% Tween-20 in TBS overnight at 4° C. Membranes were then washed 3× for 10 minutes each with 0.1% Tween-20 in TBS and incubated with secondary antibody (Cell Signaling Technology, Cat #7074) in 2.5% non-fat dry milk and 0.1% Tween-20 in TBS for 1 hour at room temperature. After 3× washes, ECL was applied (Thermofisher, Cat #32106) and proteins were detected after exposure to autoradioraphic films (Worldwide medical products, Cat #41101001) and protein intensity was quantitated using ImageJ software.

### Results:

IL1RAP has been identified as a target in multiple cancer types. Therefore, antagonistic activity of the IL1RAP mAbs in view of IL1, Il33, and IL36 signaling was assessed in both solid and non-solid cancers.

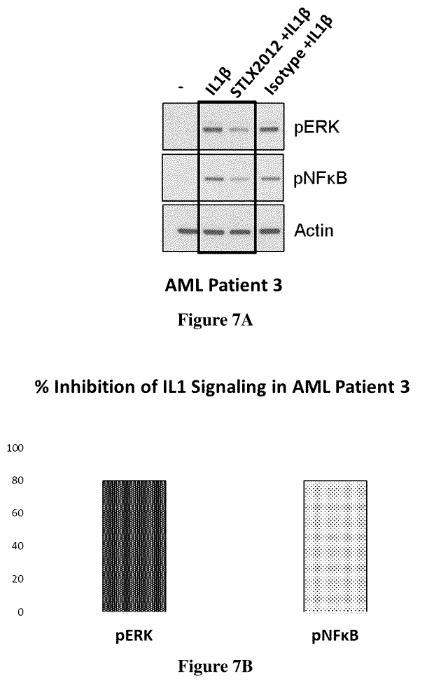

FIGS. 7A and 7B show representative results, wherein the anti-IL1RAP mAb STLX2012 inhibited IL1 signaling in AML patient samples. AML patient samples were treated with IL1β in the presence or absence of the STLX2012 IL1RAP mAb. STLX2012 IL1RAP mAb inhibited WI-induced downstream signaling, which was monitored by Western blot using phospho-specific antibodies against ERK and NFκB (FIG. 7A). Actin was used as a loading control. The bar graph of FIG. 7B presents % inhibition of phosphorylation of ERK and NFκB.

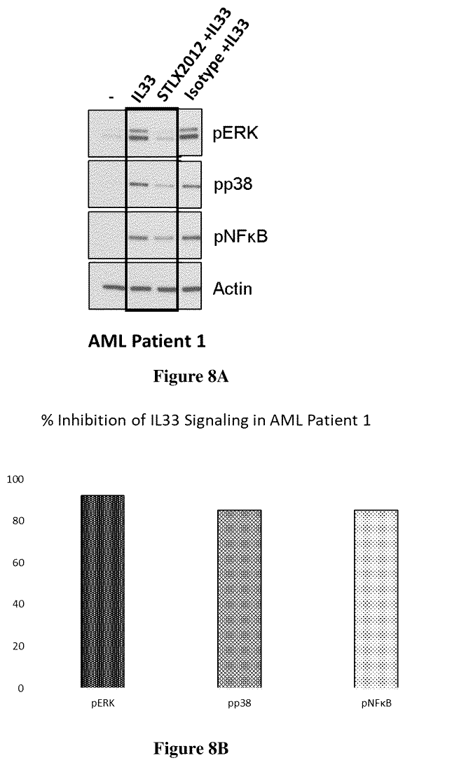

FIGS. 8A and 8B show representative results, wherein the anti-IL1RAP mAb STLX2012 inhibited IL33 signaling in AML patient samples. AML patient samples were treated with IL33 in the presence or absence of the IL1RAP STLX2012 mAb, and inhibition of IL33-induced downstream signaling was monitored by Western blot using phospho-specific antibodies against ERK, p38 and NFκB (FIG. 8A). Actin was used as a loading control. The bar graphs of FIG. 8B present % inhibition of phosphorylation of ERK, p38 and NFκB. Similar results were observed for the other patient samples (Data not shown).

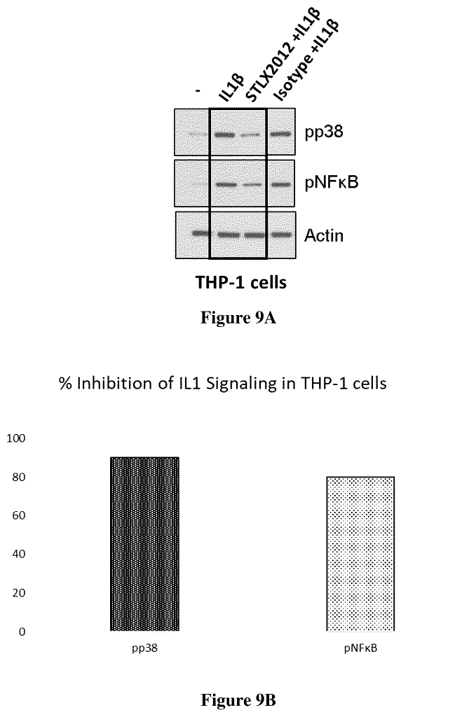

THP-1 cells are a monocyte tissue culture cell line derived from an AML patient FIGS. 9A and 9B show representative results, wherein the anti-IL1RAP mAb STLX2012 inhibited IL1 signaling in these AML (THP-1) cells. The AML cells were treated with IL1P in the presence or absence of IL1RAP mAb STLX2012, wherein the IL1RAP mAb STLX2012 inhibited I-induced downstream signaling, which was monitored by Western blot using phospho-specific antibodies against p38 and NFκB (FIG. 9A). Actin was used as a loading control. The bar graphs of FIG. 9B presents % inhibition of phosphorylation of p38 and NFκB.

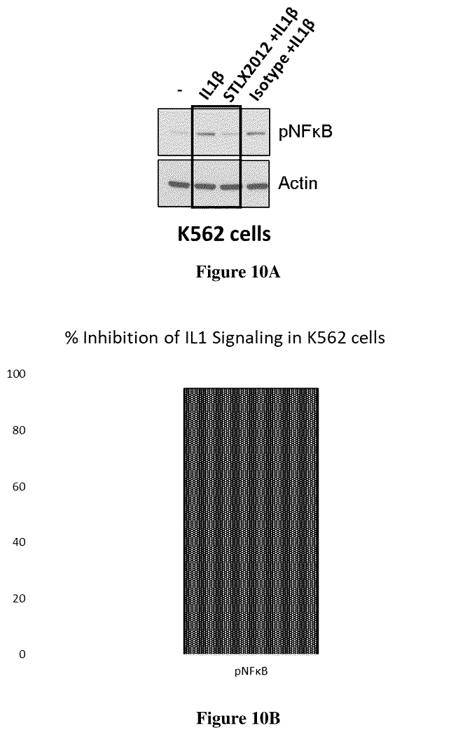

K562 cells are a lymphoblast cell line derived from a chronic myelogenous leukemia (CML) patient. FIGS. 10A and 10B show representative results, wherein the anti-IL1RAP mAb STLX2012 inhibited IL1 signaling in these CML (K562) cells. CML cells were treated with IL1β in the presence or absence of mAb STLX2012, wherein the IL1RAP mAb STLX2012 inhibited I-induced downstream signaling, which was monitored by Western blot using phospho-specific antibody against NFκB (FIG. 10A). Actin was used as a loading control. The bar graphs of FIG. 10B present % inhibition of phosphorylation of NFκB.

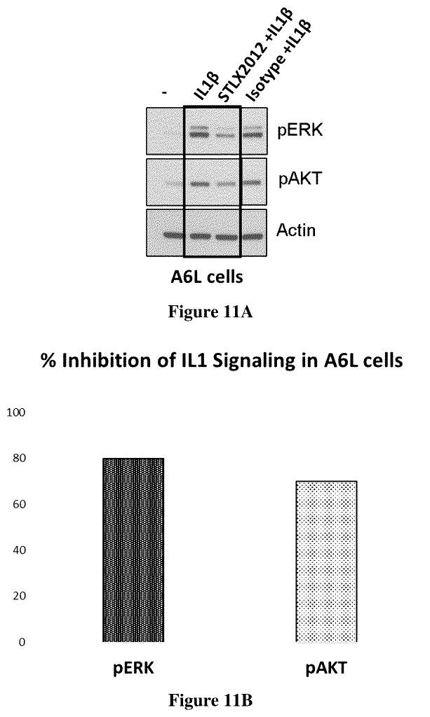

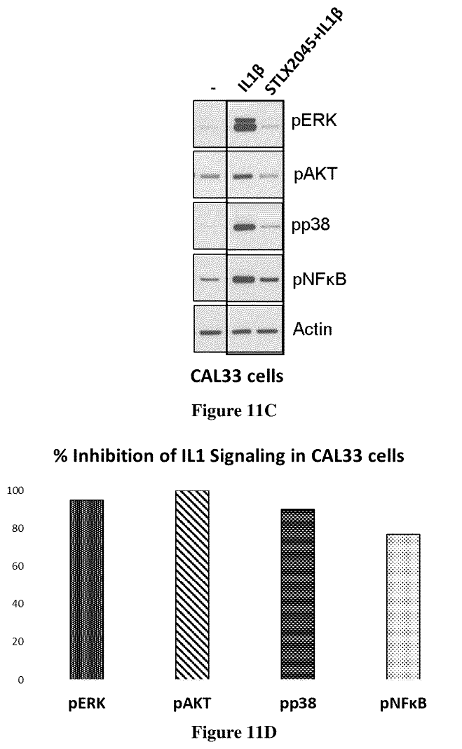

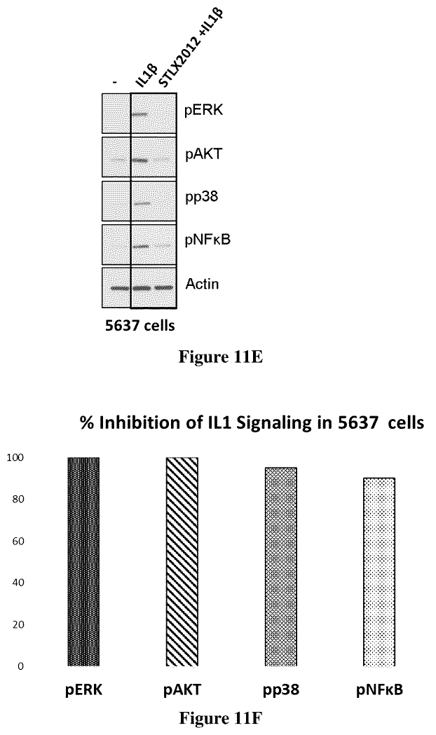

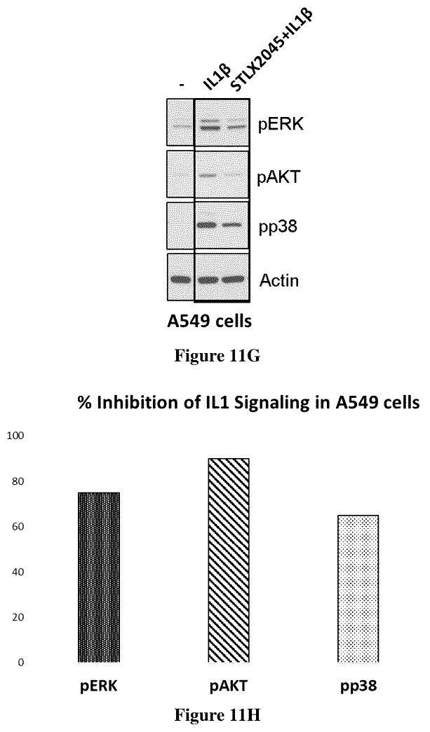

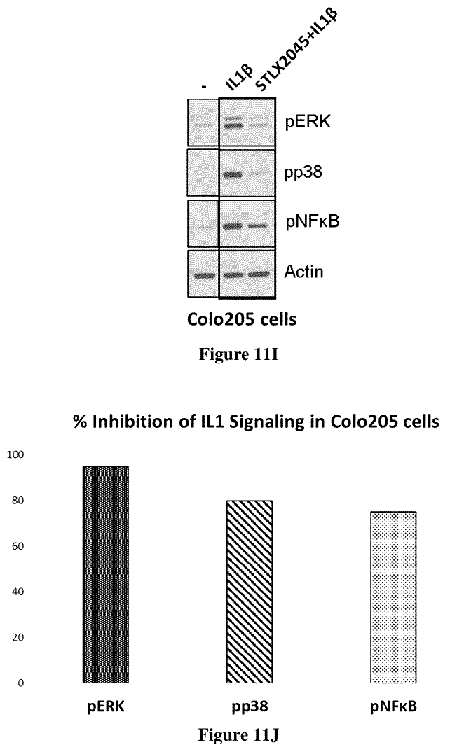

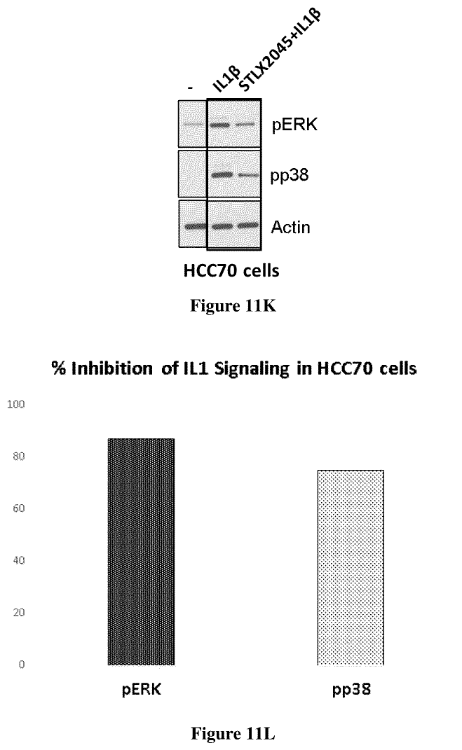

Next a range of cell lines derived from solid tumors were used to determine the ability of the IL1RAP mAbs to inhibit IL1 signaling. FIGS. 11A-11L show representative results, wherein the anti-IL1RAP mAbs STLX2012 or STLX2045 inhibited IL1 signaling in solid tumor cancer cells: Pancreatic cancer cells (A6L, https://web.expasy.org/cellosaurus/CVCL_E302) (FIGS. 11A-11B), HNSCC cells (CAL33) (FIGS. 11C-11D), bladder cancer cells (5637) (FIGS. 11E and 11F), NSCLC (A549) (FIGS. 11G and 11H), colorectal cancer cells (Colo205) (FIGS. 11I and 11J) and TNBC cells (HCC70) (FIGS. 11K and 11L). The results presented in FIGS. 11A, 11C, 11E, 11G, 11I, and 11K present data wherein the cells were treated with IL1β in the presence or absence of anti-IL1RAP mAbs STLX2012 or STLX2045. mAbs inhibited IL1-induced downstream signaling, which was monitored by Western blot using phospho-specific antibody against ERK, AKT, p38 and NFκB. Actin was used as a loading control. The bar graphs of FIGS. 11B, 11D, 11F, 11H, 11J, and 11L present % inhibition of phosphorylation of ERK, AKT, p38 and NFκB.

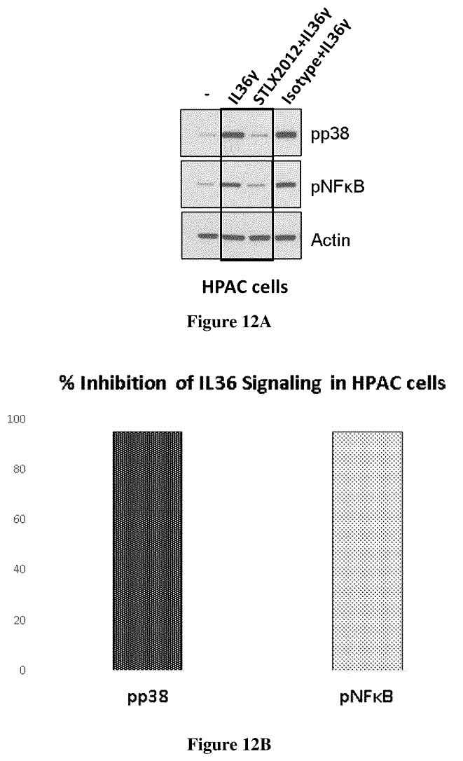

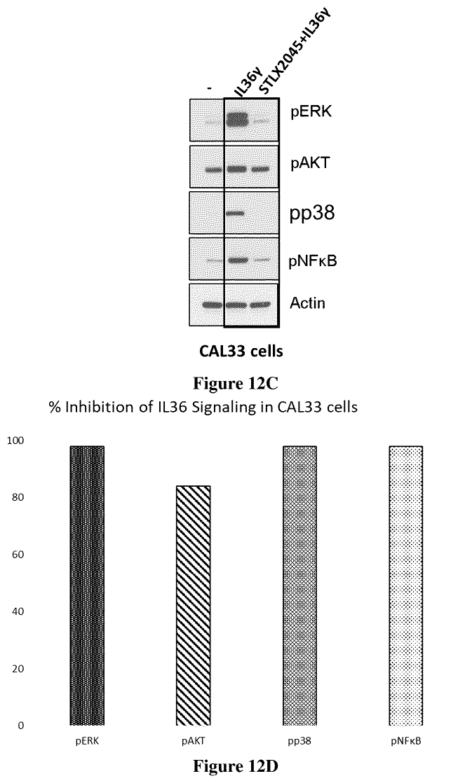

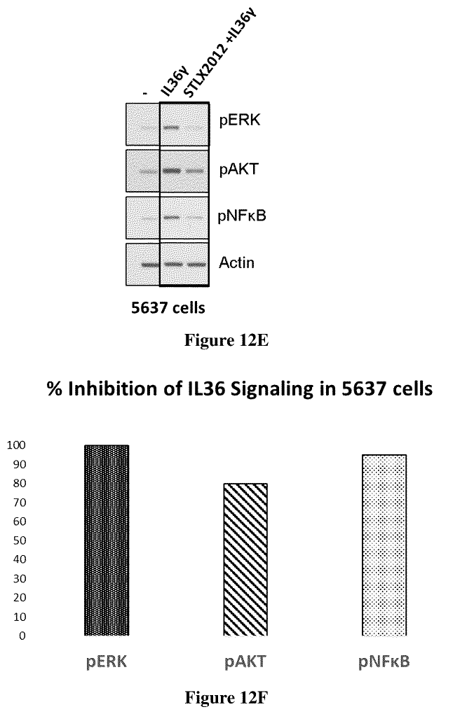

Representative cell lines derived from solid tumors were also used to determine the ability of the IL1RAP mAbs to inhibit IL36 signaling (FIGS. 12A-12F). Inhibition of IL36 signaling in solid tumor cancer cells by representative IL1RAP mAbs STLX2012 or STLX2045 was observed in Pancreatic cancer cells (HPAC) (FIGS. 12A-12B), HNSCC cells (CAL33) (FIGS. 12C-12D) and bladder cancer cells (5637) (FIGS. 12E and 12F). Cells were treated with IL36γ in the presence or absence of IL1RAP mAbs STLX2012 or STLX2045. mAbs inhibited IL36-induced downstream signaling, which was monitored by Western blot using phospho-specific antibody against ERK, AKT, p38 and NFκB (FIGS. 12A, 12C, and 12E). Actin was used as a loading control. The bar graphs of FIGS. 12B, 12D, and 12F present % inhibition of phosphorylation of ERK, AKT, p38 and NFκB.

Summary: The results shown here demonstrate the efficacy of the IL1RAP mAb to inhibit IL1, IL22, and IL36 signally across a range of solid and non-solid cancers.

### Example 5: Inhibition of Proliferation and Viability of AML Patient Samples, and Inhibition of Proliferation of Cancer Cells by IL1RAP mAbs

Objective: To examiner the effect of IL1RAP mAbs on cell proliferation and viability.

### Methods:

Inhibition of Proliferation of AML Patient Samples or Cancer Cells by IL1RAP mAbs

AML patient samples or cancer cells (AML and CML) were plated at density of 3×10³ cells/well in two 96-well plates and incubated overnight at 37° C./5% CO₂ in humidified incubator. The next day (Day 1) cells in one of the plates were treated with IL1RAP antibody at 10-150 μg/ml at a total volume of 100 μl. Plates were kept at 37° C./5% CO₂ in humified for 3-6 days. Cells treated with vehicle or IgG served as controls and every treatment was conducted in triplicates. The second plate was treated on Day 1 with media and cell proliferation was measured on the same day by CellTiter-Glo (Promega, Cat #G7572). In more details, wells were mixed with equal volume (100 μl) of CellTiter-Glo and agitated for 2 minutes followed by 10 minutes incubation at room temperature in the dark. Luminescence signal was measured in Cytation™ (Biotek) plate reader. After 3-6 days, cell proliferation in the second plates was measured separately in the same way.

Blocking of cell proliferation by antibodies was also monitored with Incucyte (Santorius). Cells were plated and treated as before, but the experiment was conducted in one plate that was kept all the time inside the Incucyte at 37° C./5% CO₂ in humified incubator. Starting from plating day and up to 7 days, Incucyte monitored cell density every day and cell proliferation was calculated (data not shown).

Inhibition of Cell Viability of Patient-Derived AML Samples by IL1RAP mAbs

Primary patient-derived AML cells collected using leukapheresis or peripheral blood draw were selected based on expression levels of IL1RAP, IL1R1 and IL1RL1. On study day 0, cell viability was calculated, and cells were seeded at a density of 20,000 viable cells/well in 96-well plates. Cells were treated on Day 0 with IL1RAP monoclonal antibodies at a concentration of 100 nM followed by 5-fold serial dilutions. Cytarabine treatment (5 μM) was used as a positive control. Plates were kept at 37° C./5% CO₂ in humified incubator and media was not changed during the duration of the assay. On Day 6, plates were removed from incubator and equilibrated to room temperature for up to 30 minutes. Then CellTiter-Glo was added to wells (100 μl) and plates were mixed for 2 minutes on plate rocker, followed by 10 minutes incubation at room temperature. Luminescent signal was recorded using Tecan plate reader and IC50s were calculated.

### Results:

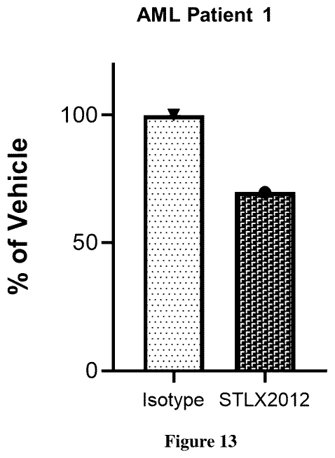

FIG. 13 shows inhibition of proliferation of AML patient samples by representative IL1RAP mAb STLX2012. AML patient samples were treated with IL1RAP mAbs (1 μM) for 7 days. IL1RAP mAb STLX2012 inhibited cell proliferation, which was measured by Cell-Titer Glo. Additional AML patient samples were used (Data not shown). Similar results were observed for the other patient samples (Data not shown).

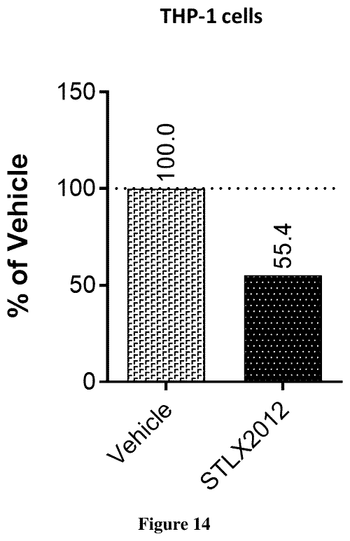

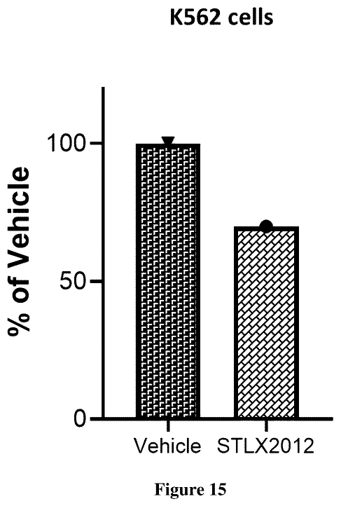

FIG. 14 shows inhibition of proliferation of AML cells by representative IL1RAP mAb STLX2012. AML cells (THP-1) were treated with IL1RAP mAbs STLX2012 (66 nM) for 4 days. IL1RAP mAbs STLX2012 inhibited cell proliferation, which was measured by Cell-Titer Glo

FIG. 15 shows inhibition of proliferation of CML cells by IL1RAP mAbs. CML cells (K562) were treated with representative IL1RAP mAb STLX2012 (333 nM) for 3 days. IL1RAP mAb STLX2012 inhibited cell proliferation which was measured by Cell-Titer Glo.

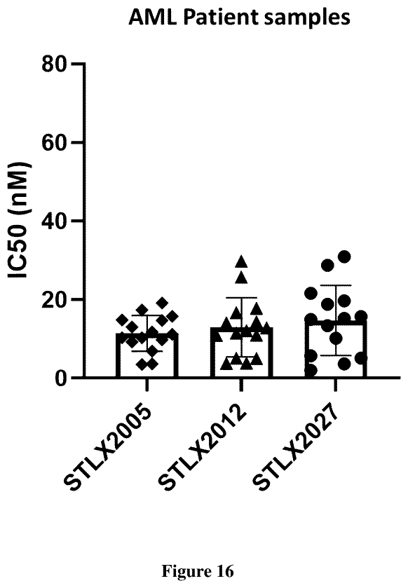

FIG. 16 shows inhibition of viability of patient-derived AML samples by representative IL1RAP mAbs STLX2005, STLX2012, and STLX2027. 15 patient-derived AML samples were treated with IL1RAP mAbs (0.1-100 nM) for 6 days, wherein the mAbs inhibited cell proliferation, which was measured by Cell-Titer Glo and IC50s were calculated. Similar results were observed for STLX2045 IL1RAP mAbs (Data not shown).

Summary: The results presented here show the IL1RAP mAbs inhibited both proliferation and viability of cancer cells.

### Example 6: Inhibition of Clonogenic Capacity of AML Patient Samples by IL1RAP mAbs

Objective: To assay the IL1RAP mAbs for the ability to inhibit clonogenic capacity of cancer cells.

### Methods:

AML patient samples or healthy control samples (4000 cells) were washed and resuspended in 30 μl Iscove's Modified Dulbecco's Medium (IMDM) media supplemented with 2% FBS. Cells (301) were mixed with 3 ml methylcellulose (MthoCult H4034 Optimum) supplemented with pen/strep or primocin (100 μg/ml) and IL1RAP antibodies (333 nM) and 1 ml were seeded in 35 mm dishes in duplicates. The dishes were incubated at 37° C./5% CO₂ in humidified incubator for 14-16 days. Colonies were counted using inverted microscope.

### Results:

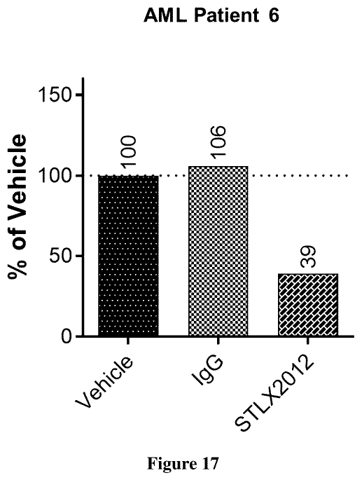

FIG. 17 shows inhibition of clonogenic capacity of AML patient samples by representative IL1RAP mAb STLX2012. AML patient samples were treated with IL1RAP mAb STLX2012 (333 nM) for two weeks, wherein the data shows that the antibodies inhibited the clonogenic capacity, which was calculated by counting the formation of colonies. Additional AML patient samples were used (Data not shown). Similar results were observed for other anti IL1RAP mAbs (Data not shown).

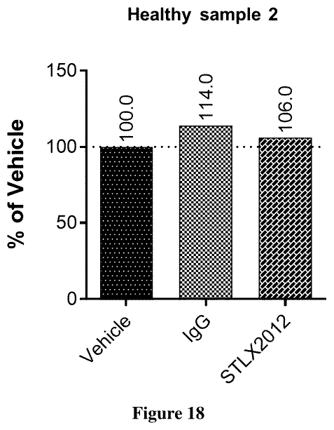

FIG. 18 shows IL1RAP mAbs do not inhibit the clonogenic capacity of healthy control samples. Healthy control samples were treated with IL1RAP mAb STLX2012 (333 nM) for two weeks, wherein, incubation with the antibodies did not affect the clonogenic capacity of healthy control samples, which was calculated by counting the formation of colonies. Additional healthy control samples were used (Data not shown). Similar results were observed for other anti IL1RAP mAbs (Data not shown).

Summary: The results presented here demonstrate that the clonogenic inhibition activity of the IL1RAP mAbs is specific for cancer cells.

### Example 7: Induction of Expression of Macrophage Differentiation Markers in AML Cells by IL1RAP mAbs

Objective: To determine if IL1RAP mAbs induced expression of macrophage differentiation markers.

Methods: AML cells (THP-1) were seeded at 0.5×10⁶/well in 6-well plate and were treated with IL1RAP antibody or IgG control at 150 μg/ml for 48 hours. After 48 hours cells were collected, and expression of differentiation markers was monitored by flow cytometry. Cells were stained for CD14 (BD, cat: 325620) and CD15 (Thermo, cat: 17-01-59-42) differentiation markers and data were analyzed with FlowJo.

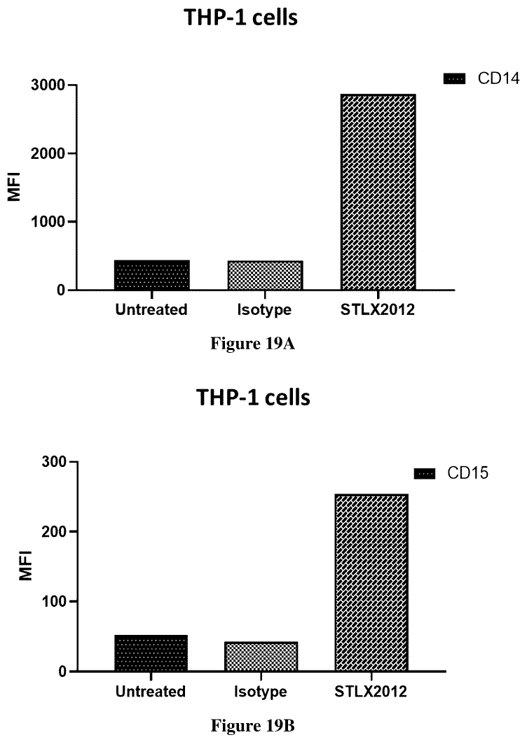

Results: FIGS. 19A and 19B show induction of expression of differentiation markers by IL1RAP mAbs. mAbs STLX2012 induced the expression of CD14 (FIG. 19A) and CD15 (FIG. 19B) differentiation markers on AML cells (THP-1).

Summary: The results presented here demonstrate that IL1RAP mAbs induced expression of macrophage differentiation markers.

### Example 8: Inhibition of IL-6 Secretion by IL1RAP mAbs

Objective: To determine if IL1RAP mAbs inhibit IL-1-induced IL-6 secretion.

Methods: A549 cells were seeded on CytoOne® 24 well plate (USA Scientific, Cat #CC7682-7524) at 0.05×10⁶/mL and were kept at 37° C./5% CO₂ in humified incubator. After 6 hours, the cells were treated with IL-1β (0.5 ng/ml) in the presence or absence of IL1RAP antibodies at 66 nM, 200 nM, 666 nM and 2 μM at 37° C./5% CO₂ in humified incubator. Cell culture media was collected after 72 hours and was centrifuged at 4° C. for 5 min. All supernatant samples were analyzed in triplicates. Human IL-6 microplate (R&D Part #890045) was incubated for 2 hours at room temperature with standard, control, or supernatant samples (100 μl) diluted in assay diluent RD1W (100 μl). Samples were aspirated and washed with wash buffer (R&D, cat #895003) four times. Human IL-6 conjugate (200p1) (R&D, cat #890046) was added to each well and incubated for 2 hours at room temperature. Aspiration/wash step was repeated as stated above. Equal volume of substrate color reagent A (R&D, cat #895000) and B (R&D, cat #895001) were mixed, and the substrate solution (200 μl) was added to each well and incubated for 20 minutes at room temperature in the dark. Stop solution (50p1) (R&D, cat #895032) was added to each well and optical density was determined at 450 nm by Cytation™ Cell Imaging Multi-Mode Reader (BioTek).

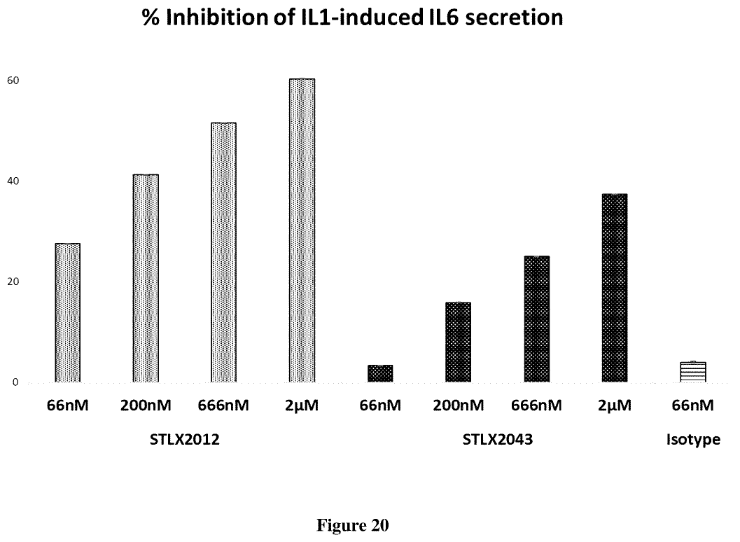

Results: FIG. 20 shows IL-1-induced IL-6 secretion by A549 cells was inhibited by mAbs STLX2012 and STLX2043 in a dose-dependent manner.

Summary: The results presented here demonstrate that IL1RAP mAbs inhibit IL-1-induced IL-6 secretion.

### Example 9: Inhibition of IL-8 Secretion by IL1RAP mAbs

Objective: To determine if IL1RAP mAbs inhibit IL-36-induced IL-8 secretion.

Methods: A431 cells were seeded on CytoOne® 24 well plate (USA Scientific, Cat #CC7682-7524) at 0.05×10⁶/mL and were kept at 37° C./5% CO₂ in humified incubator. After 6 hours the cells were treated with IL-36γ (0.5 ng/ml) in the presence or absence of IL1RAP antibodies at 66 nM, 200 nM, 666 nM and 2 M. Cell culture media was collected after 72 hours and was centrifuged at 4° C. for 5 min. All supernatant samples were analyzed in triplicates. Human IL-8 microplate (R&D cat #890462) was incubated for 2 hours at room temperature with standard, control, or supernatant samples (1001) diluted in assay Diluent RD1-85 (1001). Samples were aspirated and washed with wash buffer (R&D Part #895003) for four times. Human IL-8 conjugate (200 μl) (R&D cat #890465) was added to each well and incubated for 2 hours at room temperature. Aspiration/wash step was repeated as stated above. Equal volume of substrate color reagent A (R&D, cat #895000) and B (R&D, cat #895001) were mixed, and the substrate solution (200 ul) was added to each well and incubated for 20 minutes at room temperature in the dark. Stop solution (501) (R&D, cat #895032) was added to each well and optical density of each well was determined at 450 nm with Cytation™ Cell Imaging Multi-Mode Reader (BioTek).

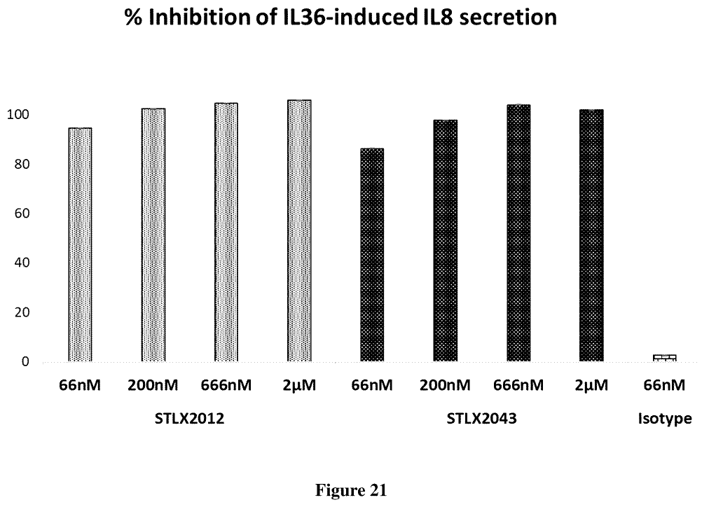

Results: FIG. 21 shows IL-36-induced IL-8 secretion by A431 cells was inhibited by mAbs STLX2012 and STLX2043 in a dose-dependent manner.

Summary: The results presented here demonstrate that IL1RAP mAbs inhibit IL-36-induced IL-8 secretion.

### Example 10: Inhibition of IL367-Induced Signaling by an IL1RAP mAb

Objective: To analyze IL1RAP mAbs inhibition of IL-36γ-induced signaling.

### Methods:

Inhibition by STLX2012 was measured by the HEKBlue-IL36 cell-based reporter assay. The HEK-Blue IL36 (Invivogen, Cat #hil36r-hkb) cell line was used to monitor NFκB activity upon stimulation with IL36γ cytokine. In this cell line, the secreted embryonic alkaline phosphatase reporter gene (SEAP) is inducibly expressed under the control of IFN-β minimal promoter fused to NFκB/AP-1. Specificity to IL36γ cytokine was achieved by blockage of TNF-α and IL1 response. Cells are maintained in the presence of zeocin (100 μg/ml) and blasticidin (10 μg/ml).

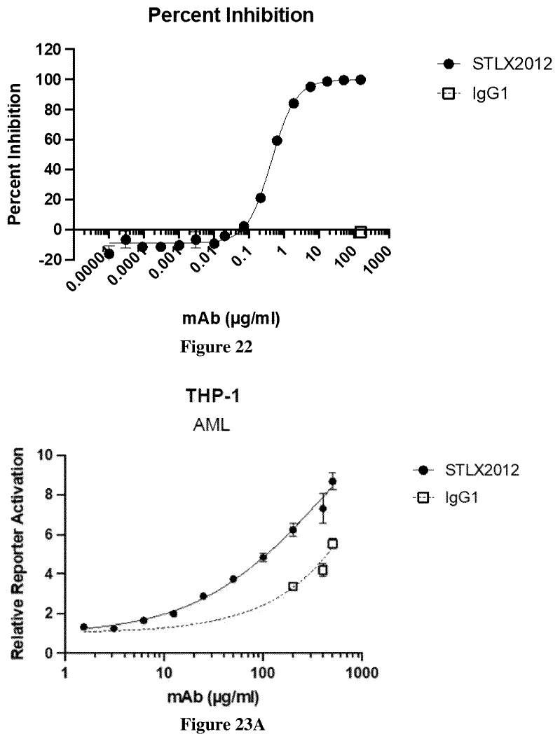

Results: FIG. 22 shows inhibition of IL36γ-induced NFκB activity by STLX2012 antibody. This representative assay of STLX2012 antibody shows inhibition of the IL36γ-induced NFκB activity in a dose-dependent manner. There was no inhibition with control IgG1 antibody.

Summary: STLX2012 inhibits IL36γ-induced signaling in a dose-dependent manner.

### Example 11: Induction of ADCC Reporter in Multiple Cell Lines and AML Patient Samples by an IL1RAP mAb

Objective: To test if IL1RAP mAb can induce Antibody-Dependent Cellular Cytotoxicity (ADCC).

Methods: ADCC activity is mediated through binding of NK cell's CD16 receptors to the Fc region of antibodies. A Jurkat-Lucia NFAT-CD16 ADCC Reporter cell assay (Invivogen, Cat #jktl-nfat-cd16; https://www.invivogen.com/jurkat-lucia-nfat-adcc-adcp-cells) was used to analyze IL1RAP mAb STLX2012 for ADCC activity. Briefly, the Jurkat cell line was engineered to express the cell surface Fc receptor CD16A (FcγRIIIA) and the Lucia luciferase reporter gene. ADCC is triggered by CD16A cross-linking upon antigen-bound antibody binding at the surface of immune effector cells. Cells are maintained in the presence of zeocin (100 μg/ml) and blasticidin (10 μg/ml).

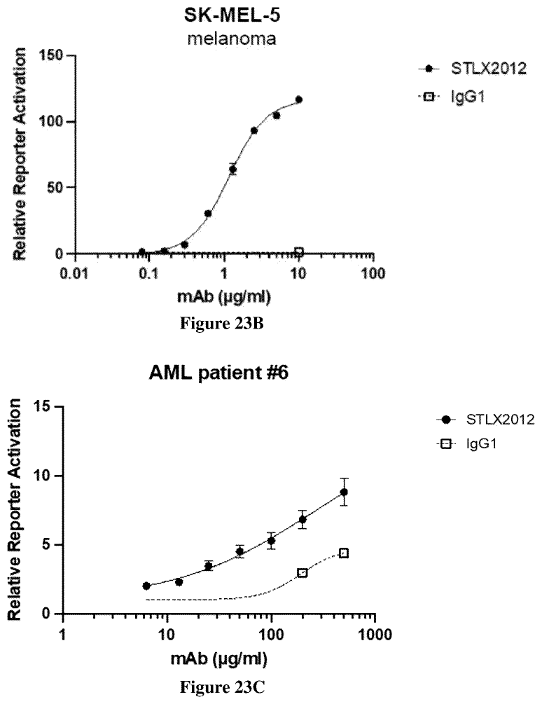

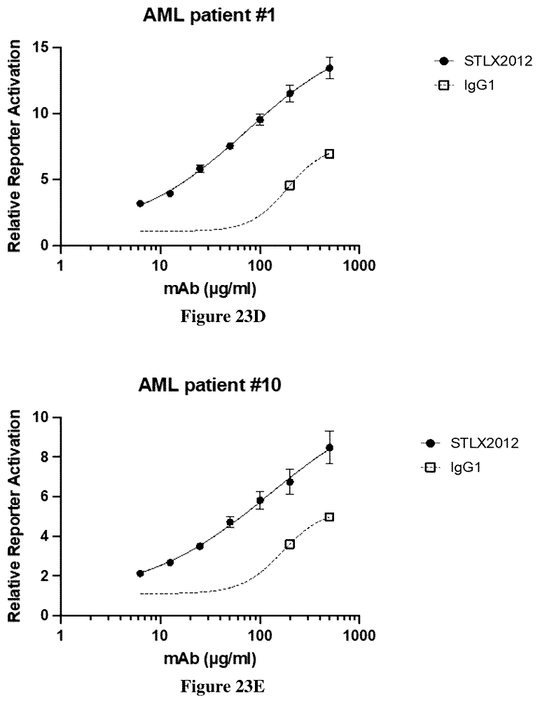

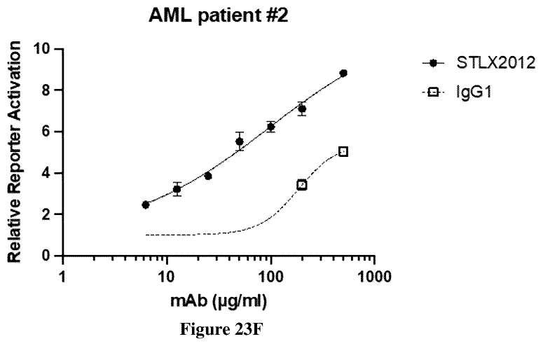

Results: FIGS. 23A-23F show that STLX2012 antibody mediated ADCC activity with a CD16-dependent reporter cell assay. Specifically, the representative assay using the STLX2012 antibody showed ADCC activity in a Jurkat-based reporter assay against THP-1 cells (AML cell line; FIG. 23A), SK-MEL-5 cells (melanoma cell line; FIG. 23B), and AML patient-derived leukemic cells (FIGS. 23C-23F).

Summary: The results here indicate that an anti-IL1RAP antibody may be used directly to cause cytotoxicity to diseased cells, for example cancer cells.

### Example 12: Enhancement of ADCC Reporter Activity by a Modified IL1RAP mAb

Objective: To determine if the ADCC activity of STLX2012 could be enhanced.

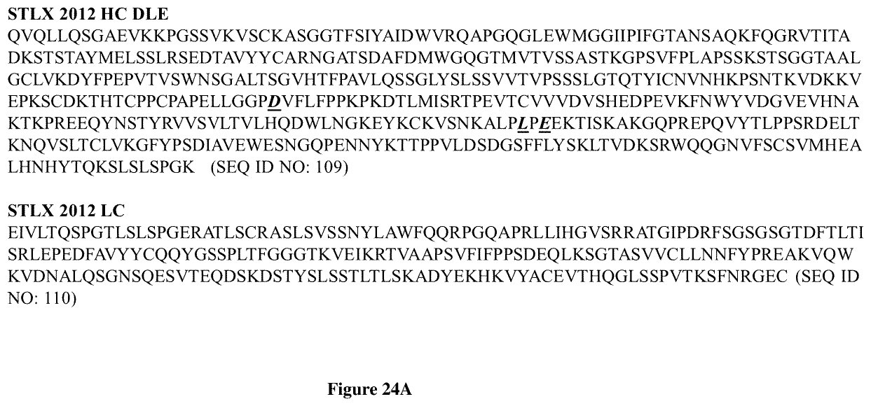

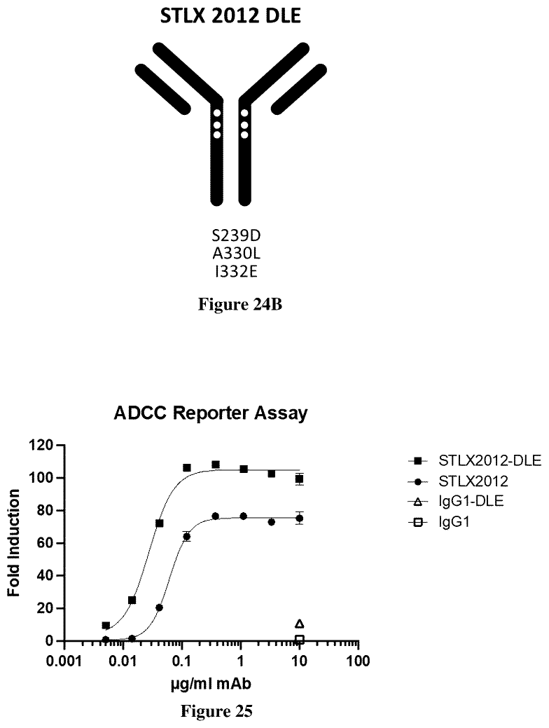

Methods: Three substitution mutations: S239D, A330L, and 1332E, were introduced into the Fc region of the STLX2012 antibody heavy chain (HC) as per Lazar et al., Engineered antibody Fc variants with enhanced effector function. Proc Natl Acad Sci USA. 2006 Mar. 14; 103(11):4005-10 and Liu et al. (2020) ibid. (FIG. 24A) The anti-IL1RAP antibody incorporating this modified heavy chain was termed STLX 2012 DLE. The positioning of these amino acid substitutions in STLX2012-DLE is shown in FIG. 24B, wherein the heavy chain is complexed with the STLX 2012 light chain (LC).

The STLX2012-DLE antibody was then analyzed for ADCC activity using the Jurkat-Lucia NFAT-CD16 ADCC Reporter cell assay, described in Example 11 above.

### Results:

The amino acid sequences of the heavy and light chains of the modified STLX2012-DLE antibody are presented below and in FIG. 24A.

STLX 2012 HC DLE:
(SEQ ID NO: 109)
QVQLLQSGAEVKKPGSSVKVSCKASGGTFSIYAIDWVRQAPGQGLEWMGG
IIPIFGTANSAQKFQGRVTITADKSTSTAYMELSSLRSEDTAVYYCARNG
ATSDAFDMWGQGTMVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPDVFLFPPKPK
DTLMISRTPEVTCVVVDVSHEDPEVKFNWYVDGVEVHNAKTKPREEQYNS
TYRVVSVLTVLHQDWLNGKEYKCKVSNKALPLPEEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
Modified (substitution mutations) amino acid
residues are shown in Bold/underlined/italics.
STLX 2012 LC:
(SEQ ID NO: 110)
EIVLTQSPGTLSLSPGERATLSCRASLSVSSNYLAWFQQRPGQAPRLLIH
GVSRRATGIPDRFSGSGSGTDFTLTISRLEPEDFAVYYCQQYGSSPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Analysis showed that the STLX2012-DLE antibody provided enhanced ADCC activity compared to original STLX2012 antibody in a Jurkat-based reporter assay against SK-MEL-5 cells (melanoma cell line). ADCC activity was measured by the Jurkat-Lucia NFAT-CD16 reporter assay. There was no activity with either control IgG1 antibody or control IgG1 containing the DLE mutation.

### Example 13: STLX2012 Antibody Inhibited Colony Formation in Combination with Azacitidine or Venetoclax

Objective: To assess an anti-IL1RAP colony formation capacity in combination with other chemotherapeutic agents.

Methods: Clonogenicity of AML patient-derived samples were assayed using STLX2012 alone or in combination with Azacitindine (Aza) or Venetoclax (Ven). AML patient-derived samples were plated in MethoCult media and cultured in the presence of STLX2012 (50 μg/mL) or control IgG1 antibody (50 μg/mL), plus or minus Azacitidine (100 nM) or Venetoclax (100 nM), for 2 weeks. Colony numbers were counted manually using an inverted microscope and normalized to the control IgG1 control.

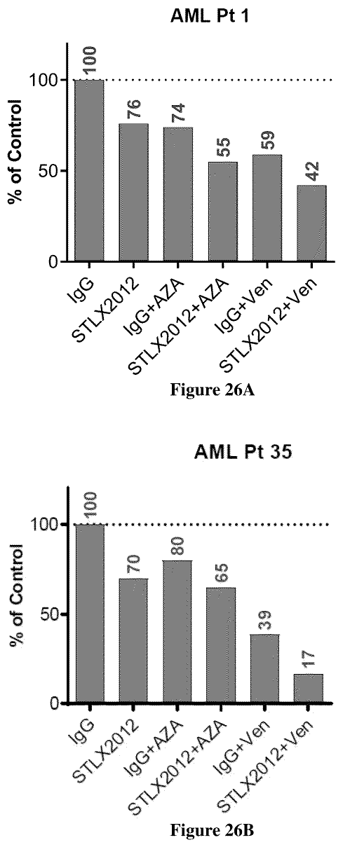

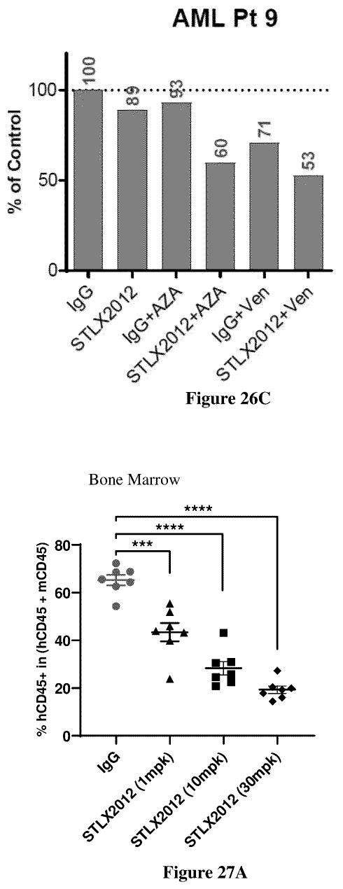

Results: FIGS. 26A-26C present representative assays of clonogenicity showing that incubation with STLX2012 antibody decreased colony numbers in AML patient samples. Further reduction in colony numbers was observed when STLX2012 antibody was combined with Azacitidine or Venetoclax.

Summary: The results presented here demonstrate that clonogenic inhibition activity of the IL1RAP mAbs may be enhanced by combination with other therapeutic cancer agents.

### Example 14: STLX2012 Antibody Inhibited AML Patient-Derived Sample Engraftment in Immunodeficient Mice

Objective: To assess an anti-IL1RAP activity in an AML mouse Xenograft model.

Methods: NSG™-SGM3 immunodeficient mice (Jackson Laboratory) were irradiated with 200 rads and then infused with AML cells (patient #6) collected from a leukapheresis sample and then treated bi-weekly with three doses of STLX2012 or control IgG1 antibody for 7 weeks. Doses were 1 mpk, 10 mpk, or 30 mpk for STLX2012, and 30 mpk for control IgG1.

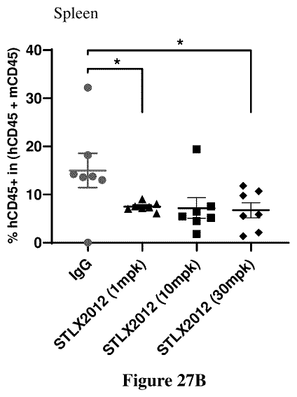

Results: On day 50, the mice treated with STLX2012 antibody had significantly reduced human CD45+AML cells in the bone marrow (FIG. 27A) and spleens (FIG. 27B) compared to mice treated with control IgG, as determined by flow cytometry. The results showed a dose-dependent reduction in bone marrow (BM) chimerism with STLX2012 treatment

Summary: The results presented here demonstrate that STLX2012 antibody can block/inhibit human AML engraftment in a xenograft model, further adding to the potential treatment in the clinical setting

While certain features of the IL1RAP antibodies, activities, and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the IL1RAP antibodies and methods of use thereof.

What is claimed is:

1. An isolated anti-IL1RAP antibody comprising three complementarity determining regions (CDRs) on a heavy chain (HCDR1, HCDR2, and HCDR3) and three CDRs on a light chain (LCDR1, LCDR2, and LCDR3), wherein
(a) the HCDR1, HCDR2, and HCDR3 comprise the amino acid sequences of SEQ ID NOs: 35, 50, and 65, and the LCDR1, LCDR2, and LCDR3 comprise the amino acid sequences of SEQ ID NOs: 77, 90, and 102; or
(b) the HCDR1, HCDR2, and HCDR3 comprise the amino acid sequences of SEQ ID NOs:33, 46 and 62, and the LCDR1, LCDR2, and LCDR3 comprise the amino acid sequences of SEQ ID NOs:74, 87, and 99; or
(c) the HCDR1, HCDR2, and HCDR3 comprise the amino acid sequences of SEQ ID NOs:33, 47, and 62, and the LCDR1, LCDR2, and LCDR3 comprise the amino acid sequences of SEQ ID NOs:75, 88, and 100; or
(d) the HCDR1, HCDR2, and HCDR3 comprise the amino acid sequences of SEQ ID NOs:34, 48, and 63, and the LCDR1, LCDR2, and LCDR3 comprise the amino acid sequences of SEQ ID NOs:76, 89, and 101; or
(e) the HCDR1, HCDR2, and HCDR3 comprise the amino acid sequences of SEQ ID NOs:35, 4,9 and 64, and the LCDR1, LCDR2, and LCDR3 comprise the amino acid sequences of SEQ ID NOs:77, 90, and 102; or
(f) the HCDR1, HCDR2, and HCDR3 comprise the amino acid sequences of SEQ ID NOs: 33, 45, and 61, and the LCDR1, LCDR2, and LCDR3 comprise the amino acid sequences of SEQ ID NOs: 73, 87, and 98; or
(g) the HCDR1, HCDR2, and HCDR3 comprise the amino acid sequences of SEQ ID NOs:35, 51, and 64, and the LCDR1, LCDR2, and LCDR3 comprise the amino acid sequences of SEQ ID NOs:77, 90, and 102; or
(h) the HCDR1, HCDR2, and HCDR3 comprise the amino acid sequences of SEQ ID NOs:36, 50, and 64, and the LCDR1, LCDR2, and LCDR3 comprise the amino acid sequences of SEQ ID NOs:78, 90, and 102; or
(i) the HCDR1, HCDR2, and HCDR3 comprise the amino acid sequences of SEQ ID NOs:37, 52, and 66, and the LCDR1, LCDR2, and LCDR3 comprise the amino acid sequences of SEQ ID NOs:79, 91, and 103; or
(j) the HCDR1, HCDR2, and HCDR3 comprise the amino acid sequences of SEQ ID NOs:38, 53, and 67, and the LCDR1, LCDR2, and LCDR3 comprise the amino acid sequences of SEQ ID NOs:80, 92, and 104; or
(k) the HCDR1, HCDR2, and HCDR3 comprise the amino acid sequences of SEQ ID NOs:39, 54, and 67, and the LCDR1, LCDR2, and LCDR3 comprise the amino acid sequences of SEQ ID NOs:81, 92, and 104; or
(l) the HCDR1, HCDR2, and HCDR3 comprise the amino acid sequences of SEQ ID NOs:40, 55, and 68, and the LCDR1, LCDR2, and LCDR3 comprise the amino acid sequences of SEQ ID NOs:82, 93, and 105; or
(m) the HCDR1, HCDR2, and HCDR3 comprise the amino acid sequences of SEQ ID NOs:41, 56, and 69, and the LCDR1, LCDR2, and LCDR3 comprise the amino acid sequences of SEQ ID NOs:83, 94, and 106; or
(n) the HCDR1, HCDR2, and HCDR3 comprise the amino acid sequences of SEQ ID NOs:42, 57, and 70, and the LCDR1, LCDR2, and LCDR3 comprise the amino acid sequences of SEQ ID NOs:84, 95, and 107; or
(o) the HCDR1, HCDR2, and HCDR3 comprise the amino acid sequences of SEQ ID NOs:43, 58, and 70, and the LCDR1, LCDR2, and LCDR3 comprise the amino acid sequences of SEQ ID NOs:85, 96, and 107; or
(p) the HCDR1, HCDR2, and HCDR3 comprise the amino acid sequences of SEQ ID NOs:44, 59, and 71, and the LCDR1, LCDR2, and LCDR3 comprise the amino acid sequences of SEQ ID NOs:73, 87, and 98; or
(q) the HCDR1, HCDR2, and HCDR3 comprise the amino acid sequences of SEQ ID NOs:44, 60, and 72, and the LCDR1, LCDR2, and LCDR3 comprise the amino acid sequences of SEQ ID NOs:86, 97, and 108.

2. The anti-IL1RAP antibody of claim 1, wherein the antibody comprises a heavy chain variable region and a light chain variable region, said heavy chain variable region and light chain variable region comprise the amino acid sequences of SEQ ID NOs: 6 and 23; SEQ ID NOs:2 and 19; SEQ ID NOs:3 and 20; SEQ ID NOs:4 and 21; SEQ ID NOs:5 and 22; SEQ ID NOs:1 and 18; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32.

3. The anti-IL1RAP antibody of claim 1, wherein the antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

4. The anti-IL1RAP antibody of claim 3, wherein said IgG is IgG1, IgG2, IgG3, or IgG4.

5. The anti-IL1RAP antibody of claim 4, said IgG comprising a modified heavy chain amino acid sequence, said modified sequence comprising S239D, A330L, and 1332E substitution mutations.

6. A composition comprising the anti-IL1RAP antibody of claim 1 and a pharmaceutically acceptable carrier.

7. An isolated polynucleotide sequence encoding the anti-IL1RAP antibody of claim 1.

8. A vector comprising the polynucleotide sequence of claim 7.

9. A host cell comprising the vector of claim 8.

10. A method of treating a disease in a subject, comprising the step of administering to the subject a composition comprising an effective amount of the anti-IL1RAP antibody, said anti-IL1RAP antibody comprising three complementarity determining regions (CDRs) on a heavy chain (HCDR1, HCDR2, and HCDR3) and three CDRs on a light chain (LCDR1, LCDR2, and LCDR3), wherein
(a) the HCDR1, HCDR2, and HCDR3 comprise the amino acid sequences of SEQ ID NOs: 35, 50, and 65, and the LCDR1, LCDR2, and LCDR3 comprise the amino acid sequences of SEQ ID NOs: 77, 90, and 102; or
(b) the HCDR1, HCDR2, and HCDR3 comprise the amino acid sequences of SEQ ID NOs:33, 46 and 62, and the LCDR1, LCDR2, and LCDR3 comprise the amino acid sequences of SEQ ID NOs:74, 87, and 99; or
(c) the HCDR1, HCDR2, and HCDR3 comprise the amino acid sequences of SEQ ID NOs:33, 47, and 62, and the LCDR1, LCDR2, and LCDR3 comprise the amino acid sequences of SEQ ID NOs:75, 88, and 100; or
(d) the HCDR1, HCDR2, and HCDR3 comprise the amino acid sequences of SEQ ID NOs:34, 48, and 63, and the LCDR1, LCDR2, and LCDR3 comprise the amino acid sequences of SEQ ID NOs:76, 89, and 101; or
(e) the HCDR1, HCDR2, and HCDR3 comprise the amino acid sequences of SEQ ID NOs:35, 4,9 and 64, and the LCDR1, LCDR2, and LCDR3 comprise the amino acid sequences of SEQ ID NOs:77, 90, and 102; or
(f) the HCDR1, HCDR2, and HCDR3 comprise the amino acid sequences of SEQ ID NOs: 33, 45, and 61, and the LCDR1, LCDR2, and LCDR3 comprise the amino acid sequences of SEQ ID NOs: 73, 87, and 98; or
(g) the HCDR1, HCDR2, and HCDR3 comprise the amino acid sequences of SEQ ID NOs:35, 51, and 64, and the LCDR1, LCDR2, and LCDR3 comprise the amino acid sequences of SEQ ID NOs:77, 90, and 102; or
(h) the HCDR1, HCDR2, and HCDR3 comprise the amino acid sequences of SEQ ID NOs:36, 50, and 64, and the LCDR1, LCDR2, and LCDR3 comprise the amino acid sequences of SEQ ID NOs:78, 90, and 102; or
(i) the HCDR1, HCDR2, and HCDR3 comprise the amino acid sequences of SEQ ID NOs:37, 52, and 66, and the LCDR1, LCDR2, and LCDR3 comprise the amino acid sequences of SEQ ID NOs:79, 91, and 103; or
(j) the HCDR1, HCDR2, and HCDR3 comprise the amino acid sequences of SEQ ID NOs:38, 53, and 67, and the LCDR1, LCDR2, and LCDR3 comprise the amino acid sequences of SEQ ID NOs:80, 92, and 104; or
(k) the HCDR1, HCDR2, and HCDR3 comprise the amino acid sequences of SEQ ID NOs:39, 54, and 67, and the LCDR1, LCDR2, and LCDR3 comprise the amino acid sequences of SEQ ID NOs:81, 92, and 104; or
(l) the HCDR1, HCDR2, and HCDR3 comprise the amino acid sequences of SEQ ID NOs:40, 55, and 68, and the LCDR1, LCDR2, and LCDR3 comprise the amino acid sequences of SEQ ID NOs:82, 93, and 105; or
(m) the HCDR1, HCDR2, and HCDR3 comprise the amino acid sequences of SEQ ID NOs:41, 56, and 69, and the LCDR1, LCDR2, and LCDR3 comprise the amino acid sequences of SEQ ID NOs:83, 94, and 106; or
(n) the HCDR1, HCDR2, and HCDR3 comprise the amino acid sequences of SEQ ID NOs:42, 57, and 70, and the LCDR1, LCDR2, and LCDR3 comprise the amino acid sequences of SEQ ID NOs:84, 95, and 107; or
(o) the HCDR1, HCDR2, and HCDR3 comprise the amino acid sequences of SEQ ID NOs:43, 58, and 70, and the LCDR1, LCDR2, and LCDR3 comprise the amino acid sequences of SEQ ID NOs:85, 96, and 107; or
(p) the HCDR1, HCDR2, and HCDR3 comprise the amino acid sequences of SEQ ID NOs:44, 59, and 71, and the LCDR1, LCDR2, and LCDR3 comprise the amino acid sequences of SEQ ID NOs:73, 87, and 98; or
(q) the HCDR1, HCDR2, and HCDR3 comprise the amino acid sequences of SEQ ID NOs:44, 60, and 72, and the LCDR1, LCDR2, and LCDR3 comprise the amino acid sequences of SEQ ID NOs:86, 97, and 108.

11. The method of claim 10, wherein the antibody comprises a heavy chain variable region and a light chain variable region, said heavy chain variable region and light chain variable region comprise the amino acid sequences of SEQ ID NOs: 6 and 23; SEQ ID NOs:2 and 19; SEQ ID NOs:3 and 20; SEQ ID NOs:4 and 21; SEQ ID NOs:5 and 22; SEQ ID NOs:1 and 18; SEQ ID NOs:6 and 22; or SEQ ID NOs:7 and 22; SEQ ID NOs:8 and 24; SEQ ID NOs:9 and 25; SEQ ID NOs:10 and 26; SEQ ID NOs:11 and 27; SEQ ID NOs:12 and 28; SEQ ID NOs:13 and 29; SEQ ID NOs:14 and 30; SEQ ID NOs:15 and 31; SEQ ID NOs:16 and 18; or SEQ ID NOs:17 and 32.

12. The method of claim 10, wherein the antibody comprises an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

13. The method of claim 12, wherein said IgG is IgG1, IgG2, IgG3, or IgG4.

14. The method of claim 10, wherein the disease comprises a cancer or tumor, an autoimmune disease, or GvHD.

15. The method of claim 14, wherein the cancer or tumor comprise a hematological cancer, a solid cancer, or a solid tumor.

16. The method of claim 15, wherein the hematological cancer is leukemia, lymphoma, myeloma, acute myeloid leukemia (AML), acute promyelocytic leukemia, erythroleukemia, biphenotypic B myelomonocytic leukemia, or myelodysplastic syndromes (MDS).

17. The method of claim 15, wherein said solid cancer or solid tumor is sarcoma, osteosarcoma, squamous cell carcinoma of the head and neck, Non-small-cell lung carcinoma, bladder cancer, pancreatic cancer, or pancreatic ductal adenocarcinoma.

18. The method of claim 10, wherein said disease comprises an autoimmune disease.

19. The method of claim 10, wherein said disease comprises GvHD.

20. A method of producing an anti-IL1RAP antibody, wherein said method comprises expressing the vector of claim 8 in a host cell under conditions conducive to expressing said vector in said host cell, thereby producing the anti-IL1RAP antibody.
